(19) United States
(12) Patent Application Publication
(10) Pub. No.: US 2025/0368963 A1
(43) Pub. Date: Dec. 4, 2025

(54) PROXIMAL-BIASED KIDNEY ORGANOIDS

(71) Applicant: UNIVERSITY OF SOUTHERN CALIFORNIA, Los Angeles, CA (US)

(72) Inventors: Nils Lindstrom, Los Angeles, CA (US); Jack Schnell, Los Angeles, CA (US)

(21) Appl. No.: 19/223,807

(22) Filed: May 30, 2025

Related U.S. Application Data
(60) Provisional application No. 63/654,253, filed on May 31, 2024.

(51) Int. Cl. C12N 5/071

(52) U.S. Cl. CPC C12N 5/0686; C12N 2501/727; C12N 2503/04

(57) ABSTRACT

Disclosed herein is a proximal-biased kidney organoid comprising proximal tubule cells and methods of making and using the same.

### CROSS-REFERENCE TO RELATED APPLICATIONS

This application claims the benefit of priority to U.S. Provisional Application No. 63/654,253, filed May 31, 2024, which is incorporated by reference herein in its entirety.

### BACKGROUND

The kidney maintains body fluid homeostasis by reabsorbing essential compounds and excreting waste. Proximal tubule cells, crucial for renal reabsorption of a range of sugars, ions, and amino acids, are highly susceptible to damage, leading to pathologies necessitating dialysis and kidney transplants. While human pluripotent stem cell-derived kidney organoids are used for modeling renal development, disease, and injury, the formation of proximal nephron cells in these 3D structures is incomplete. Proximal nephron cells are the most abundant cells in the human kidney, are responsible for reabsorbing 65% of the nephron filtrate, and their pathologies are the primary reason patients require dialysis and kidney transplants. In spite of their clinical importance, proximal tubule disease mechanisms are poorly understood, and human cell models are required to scrutinize disease origins and etiology. Stem cell-derived human kidney models recapitulating proximal tubule functions would therefore provide a critical tool for studying renal disease and develop therapeutic approaches.

Thus, there exists a need for such models. These needs and others are at least partially satisfied by the present disclosure.

### SUMMARY

Disclosed herein are methods which can drive the development of proximal tubule precursors in kidney organoids. Transient manipulation of the PI3K signaling pathway can activate Notch signaling in the early nephron and can drive nephrons toward a proximal precursor state. These “proximal-biased” (PB) organoid nephrons can proceed to generate proximal nephron precursor cells. Single-cell transcriptional analyses across the organoid nephron differentiation, comparing control and PB types, can confirm the role of transient Notch signaling for proximal development. Indicative of functional maturity, PB organoids can demonstrate dextran and albumin uptake, akin to in vivo proximal tubules. Moreover, PB organoids can be highly sensitive to nephrotoxic agents, display an injury response, and drive expression of HAVCR1/KIM1, an early proximal-specific marker of kidney injury. The PB organoid model therefore can have functional relevance and potential for modeling mechanisms underpinning nephron injury. These advances can improve the use of iPSC-derived kidney organoids as tools to understand developmental nephrology, model disease, test therapeutics, and for understanding human renal physiology.

In an aspect, provided is a proximal-biased kidney organoid, including a kidney organoid exposed to a PI3 kinase (PI3K) inhibitor and including proximal tubule cells, wherein incubation with the PI3K inhibitor can provide a proximal bias in the kidney organoid.

In another aspect, provided is a method of producing any of the disclosed proximal-biased kidney organoids, the method comprising incubating a plurality of precursor cells with a cell culture media, a PI3K inhibitor, and at least one compound for inducing kidney cell differentiation.

In yet another aspect, provided is a method of modeling a kidney disease, the method including: a) providing any of the disclosed proximal-biased kidney organoids; and b) observing the proximal-biased kidney organoid over a period of time.

In yet still another aspect, provided is a method of screening for therapeutic agents that modulate injury to proximal tubule cells, the method including: a) providing any of the disclosed proximal-biased kidney organoids; b) administering a therapeutic agent to the proximal-biased kidney organoid; and c) observing the proximal-biased kidney organoid over a period of time.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF DRAWINGS

FIGS. 1A-1F depicts comparative morphological and transcriptomic analyses of proximal nephron formation in vivo and in iPSC-derived kidney organoids. FIGS. 1A-1B show immunofluorescent antibody stains at defined stages of nephrogenesis. Human kidneys are from week 16 of development. FIG. 1A represents stages before the onset of HNF4A, while FIG. 1B represents the commencement and elongation of the HNF4A⁺ domain. White arrowheads indicate earliest forming HNF4A⁺ cells. Scale bars: 10 microns. FIGS. 1C-1D show whole-mount immunofluorescent stains of kidney organoids nephrons. Data in FIG. 1C match proteins and stages in FIG. 1A, while FIG. 1D corresponds to FIG. 1B. White arrowheads indicate earliest forming HNF4A⁺ cells. Scale bars: 10 microns. FIG. 1E shows ex vivo cultures of dissected E12.5 mouse kidneys in different 48 hr. culture conditions. White arrowheads indicate Hnf4a⁺ cells. Scale bars: 20 microns. FIG. 1F shows hierarchical clustering from bulk RNA-sequencing of kidney organoids at differentiation days 10, 12 (control) and 12 (treated for 48 hours with 10 μM LY294002). Genes shown are those that are highlighted in FIG. 2I. Legend categorizes genes and timepoints. Timeline of organoid differentiation and those profiled is shown in the top right.

FIGS. 2A-2L depict comparative transcriptomics and analyses of in vivo and early in vitro-forming nephrons. FIG. 2A shows single-cell RNA-sequencing of nephrogenic and ureteric lineages from week 14 and 17 developing human kidneys. FIG. 2B is a dot plot with identifying genes for each cluster. Arrows, line colors, and cluster numbers match to FIG. 2A and FIG. 2B. FIG. 2C shows feature plots for select genes. Insets highlight transition from pretubular aggregate to later nephron stages and specified gene dynamics. FIG. 2D shows single-cell RNA-sequencing from four kidney organoid datasets. Displayed are only nephrogenic cells ranging between day 7 to day 29 differentiation timepoints. FIG. 2E is a dot plot with identifying genes per cluster. FIG. 2F shows feature plots for select genes as shown for in vivo in FIG. 2C. FIG. 2G shows a whole-mount immunofluorescent stain of day 10 kidney organoids. Inset zooms in to a cluster of 4 individual organoid nephrons. Scale bars: 250 microns, and 10 microns (inset). FIG. 2H shows a whole-mount immunofluorescent stain of an individual kidney organoid nephron at day 10, with split channels. Scale bar: 10 microns. FIG. 2I shows hierarchical clustering from bulk RNA-sequencing of kidney organoids at differentiation days 10, 12 (control) and 12 (treated for 48 hours with 10 μM LY294002). Samples were averaged from n=3 biological replicates for each timepoint. Clustered genes represent those with a transcripts per million (TPM) value of greater than or equal to 25 in at least one of the samples, (6703 genes). Representative genes for each cluster are shown on the side, along with a Z-score legend. FIGS. 2J-2L show gene set enrichment analysis plots from Day 12 LY294002-treated/Day 12 control kidney organoids. Plots depict pathway effects from treatment with the PI3K inhibitor LY294002: downregulated (PI3K-Akt signaling pathway (FIG. 2J), upregulated (Notch signaling pathway (FIG. 2K), and unaffected (Hedgehog signaling pathway (FIG. 2L). Normalized enrichment scores (NES), false discovery rates (FDR), and p-values are shown for each.

FIGS. 3A-3F depict gene expression during proximal nephron formation in human nephrons kidney organoids. FIG. 3A is a dot plot of genes from an in vivo Pearson correlation test showing upregulation with human HNF1B (bottom), both HNF1B and HNF4A (middle), and just HNF4A (top). Clusters depict proximalizing lineages from the human nephron (left) and four kidney organoid datasets (right) using standard protocols. Green highlight denotes genes not upregulated in proximalizing organoid cells, red marks genes expressed in the distal nephron, and bolded genes are referenced in FIGS. 8A-8G. FIG. 3B is a Venn diagram showing the intersection of gene lists from genes enriched in the adult proximal tubule, and genes significantly downregulated in the Hnf4a-mutant mouse (257 genes, padj<0.01, log₂FC≥1.5). Intersect categorizes Hnf4a-dependent genes enriched in development (124, right) and those enriched in development and adults (133, intersect). FIGS. 3C-3D show heatmaps of human orthologs (generated using Ensembl BioMart) corresponding to the 133 mouse genes enriched in development and adult FIG. 3C, and the 124 mouse genes enriched in development FIG. 3D from FIG. 3B. Heatmaps indicate the percentage of cells expressing each gene in HNF4A⁺ clusters from human single-cell RNA-sequencing (FIG. 2A) and kidney organoid nephron single-cell RNA-sequencing from multiple sources (FIG. 2D). Scales at the bottom depict whether a gene is expressed at a higher percentage in human cells (blue) or in organoid nephron cells (red). Bolded genes are referenced in feature plots in FIG. 3E and FIG. 3F. FIGS. 3E-3F show feature plots displaying representative genes for each heatmap. Human insets highlight the transition from pretubular aggregate to later proximal nephron stages, while the organoid inset shows the proximalizing branch.

FIGS. 4A-4I depict that PI3K inhibitor-treated kidney organoid nephrons exhibit a shift toward proximal cell fates. FIG. 4A shows a whole-mount immunofluorescent stain of day 12 control and LY294002-treated kidney organoids. Insets highlight JAG1 and HNF1B protein expression. Scale bars: 500 microns. FIG. 4B shows measurements from FIG. 4A for JAG1⁺ and HNF1B⁺ nephron size (μm²) and HNF1B⁺ intensity (relative fluorescent units) for n=3 day 12 organoids. SEM error bars are shown. Statistical significance is determined using Student's t test. FIG. 4C shows bulk RNA-sequencing (TPM) of select genes on days 10, 12, 14, and 18. SEM error bars are shown from n=2 whole organoids. FIG. 4D shows a whole-mount immunofluorescent stain of control and proximal-biased day 14 organoids with insets showing HNF4A expression. Scale bars: 200 microns. FIG. 4E shows immunofluorescent antibody stains of week 16 human kidneys during HNF4A⁺, HNF4G⁺ proximal tubule elongation. White arrowheads indicate autofluorescence from endothelial cells. Scale bars: 10 microns. FIG. 4F shows a whole-mount immunofluorescent stain of control and proximal-biased day 18 organoids with insets showing HNF4A expression. Scale bars: 200 microns. FIG. 4G shows a quantification of total number of HNF4A⁺ organoid nephron segments from n=4 whole day 18 organoids. SEM error bars are shown. Statistical significance is determined using Student's t test. FIG. 4H shows a quantification of the average area (μm²) of HNF4A⁺ organoid nephron segments from n=3 whole day 18 organoids. SEM error bars are shown. Statistical significance is determined using Student's t test. FIG. 4I shows area sum (μm²) quantification of HNF4A⁺ organoid nephron segments from n=3 whole day 18 organoids.

FIGS. 5A-5D depict immunofluorescent analyses of inhibitor-treated kidney organoids. FIG. 5A shows a whole-mount immunofluorescent stain of organoid nephrons on day 12 following 48 hr. culture in control conditions (untreated), LY294002-treated, DAPT-treated, and LY294002⁺ DAPT-treated. Insets highlight clusters of forming organoid nephrons, with split channels. Scale bars: 10 microns. FIG. 5B shows whole-mount immunofluorescent stains of days 13 and 14 control and LY294002-treated (from days 10-12, proximal-biased) organoids. Scale bars: 500 microns. FIG. 5C shows a quantification of central and peripheral HNF4A⁺ and POU3F3⁺ organoid nephrons for control or proximal-biased conditions. The yellow line represents the organoid diameter, with the yellow circle marking the center of the organoid, and its diameter representing the radius of the whole organoid. Blue counts denote peripheral organoid nephrons, while red counts indicate central organoid nephrons. Organoids shown for this panel are the same that were profiled from FIG. 4F. Scale bars: 200 microns. FIG. 5D shows percent contribution quantifications derived from FIG. 5C. Bar graphs quantify structures out of the total number of central or peripheral structures as either HNF4A⁺ or POU3F3⁺. Data are averaged from n=3 biological replicate kidney organoids.

FIGS. 6A-6L depict extended single-cell transcriptomics-driven analyses and comparison of kidney organoid nephrons with in vivo human nephrons. FIG. 6A is a schematic of single-cell RNA-sequencing timepoints with UMAP reduction of kidney organoid cells, colored by samples. FIG. 6B shows a UMAP reduction of cell clusters in kidney organoids, colored by clusters. FIG. 6C is a dot plot with identifying genes per cluster for kidney organoid nephrogenic cells in FIG. 7B. FIG. 6D shows a UMAP reduction of kidney organoid nephrogenic cells with podocytes removed, used for pseudotime trajectory analysis. FIG. 6E shows a pseudotime trajectory of organoid nephron cells (with podocytes removed) from day 10 to day 18 proximal cells. FIG. 6F shows feature plots for representative genes marking different developmental stages from the pseudotime trajectory analysis. FIG. 6G shows single-cell RNA-sequencing UMAP reduction of human fetal nephron cells (FIG. 2A) merged with kidney organoid nephrogenic cells (FIG. 7B), colored by cluster. FIG. 6H shows a UMAP reduction from FIG. 6G colored by sample. FIG. 6I is a percent contribution bar graph quantifying the contribution of different original sample identities to various clusters of integrated nephrogenic cells from FIG. 6G. FIG. 6J is a dot plot with representative genes for each cluster for human-organoid merged data in FIG. 6G. FIG. 6K shows masking (red) of WT1⁺ organoid nephron segments from control and proximal-biased organoids on days 12 and 18 of differentiation. Scale bars: 200 microns. FIG. 6L shows a quantification of the average size (μm²) of WT1⁺ organoid nephron segments from control and proximal-biased organoids on days 12 and 18 of differentiation, from FIG. 6K. Data are quantified from n=2 biological replicates each. SEM error bars are shown. Statistical significance is determined using a Mann-Whitney U test.

FIGS. 7A-7G depict single-cell transcriptomic landscape and morphological changes in proximal-biased kidney organoids across differentiation stages. FIG. 7A is a schematic of single-cell RNA-sequencing timepoints with UMAP reduction of kidney organoid nephrogenic cells, colored by samples. FIG. 7B shows a UMAP reduction of nephrogenic cell clusters in kidney organoids, colored by clusters. FIG. 7C shows feature plots for select nephron marker genes with insets separating control from proximal-biased cells, and percent quantification of total cells expressing the gene. FIG. 7D shows whole-mount immunofluorescent stains of control kidney organoid nephrons from differentiation days 11, 12, 14, 15, and 18 with the bottom row highlighting HNF4A protein expression. Scale bars: 10 microns. FIG. 7E shows whole-mount immunofluorescent stains of proximal-biased kidney organoid nephrons from the same differentiation days as FIG. 7D, showing HNF4A protein expression in the bottom row. Scale bars: 10 microns. FIG. 7F shows a differentiation model for control kidney organoids based on FIG. 7D. FIG. 7G shows a differentiation model for proximal-biased kidney organoids derived from FIG. 7E.

FIGS. 8A-8G depict single-cell transcriptomics-driven comparison of in vitro human proximalization with proximal-biased kidney organoid nephrons and proximal tubule-enhanced kidney organoids. FIG. 8A shows single-cell RNA-sequencing of differentiation day 13⁺14 (27) proximal tubule-enhanced kidney organoids. FIG. 8B is a dot plot with identifying genes for each cluster. FIG. 8C shows feature plots for select genes, showing a matching comparison to FIG. 2C and FIG. 2F. FIG. 8D is a schematic of merging single-cell RNA-sequencing datasets: in vivo developing human nephrons from week 14 and 17 kidneys (FIG. 2A), proximal-biased kidney organoids from differentiation days 10, 12, 14, and 18 (FIGS. 7A-7B), and differentiation day 13⁺14 (27) proximal-tubule enhanced kidney organoids in FIG. 8A. FIG. 8E is a diagram of differentiation and sampling timelines for proximal-biased kidney organoids and proximal tubule-enhanced kidney organoids. FIG. 8F shows violin plots of representative early proximal tubule genes (expressed in human clusters 21, 24, and 20, FIG. 2A), split by dataset of origin. FIG. 8G shows violin plots of representative late proximal tubule genes (not expressed in human cluster 21, expressed in clusters 24 and 20, FIG. 2A), split by dataset of origin.

FIGS. 9A-9D depict dynamic imaging, functional profiling, and reproducibility of kidney organoid proximal biasing. FIG. 9A shows comparative live imaging of HNF4A-YFP reporter kidney organoids for control vs. proximal-biased conditions, spanning differentiation days 10 to 18. Scale bars: 200 microns. FIG. 9B shows live imaging of HNF4A-YFP kidney organoids on differentiation day 18 with TRITC-albumin uptake. Quantification of individual uptake events displayed in lower left, and insets detail HNF4A-YFP and TRITC-albumin distinction. Scale bars: whole organoid—500 microns, insets—100 microns. FIG. 9C shows quantitative data from organoid nephron single-cell RNA-sequencing (FIGS. 7A-7B) showing cell counts in HNF4A⁺ proximal clusters, categorized by control or proximal-biased conditions, expressing select genes. FIG. 9D shows whole-mount immunofluorescence comparing control and proximal-biased day 18 kidney organoids from an alternate iPSC line. Scale bars: 10 microns.

FIGS. 10A-10G depicts functional characterization and injury response in proximal-biased kidney organoids. FIG. 10A shows live imaging of HNF4A-YFP kidney organoids on differentiation day 18 with Alexa 647-conjugated dextran uptake. Quantification of individual uptake events displayed in lower left, and insets detail HNF4A-YFP and dextran distinction. Scale bars: whole organoid—500 microns, insets—100 microns. FIG. 10B shows whole-mount immunofluorescence of day 18 control and proximal-biased kidney organoids following dextran uptake assay. Boxed region is magnified and split by individual channels. Scale bars: 10 microns. FIG. 10C shows whole-mount immunofluorescence of uninjured and cisplatin-injured kidney organoids on differentiation day 21, split by control and proximal-biased conditions. Boxed area magnifies HNF4A⁺ proximal organoid nephron regions. Scale bars: 10 microns. FIG. 10D shows a timeline schematic of kidney organoid injury strategy. FIG. 10E shows a quantification of relative protein signal from HAVCR1⁺ regions in control and proximal-biased kidney organoid nephrons on day 21 following injury. Data are averaged from n=3 independent and consistent imaging panels. SEM error bars are shown. Statistical significance is determined using Student's t test. FIG. 10F shows a quantification of HNF4A⁺ proximal organoid nephron segments with HAVCR1 expression for each of the four conditions from FIG. 10C. Positive segments are counted from a 20× widefield panel of 4 images per organoid, from n=2 biological replicate organoids for each condition. SEM error bars are shown. Statistical significance is determined using Student's t test. FIG. 10G shows whole-mount immunofluorescence of uninjured and cisplatin-injured proximal-biased kidney organoid nephrons on day 21. Yellow arrows denote cells of the HNF4A⁺ organoid nephron tubule with low HNF4A protein expression and positive expression of γH2AX. White arrows indicate cells with high HNF4A, and low γH2AX detection. Split channels separate HNF4A and γH2AX for cisplatin-injured condition. Scale bars: 10 microns.

FIG. 11 depicts a comparison of PI3K inhibitors LY94002 and GDC-0941.

FIGS. 12A-12H depict single-nucleus RNA-sequencing analysis of day 21 double-dose cisplatin-treated (injured) and uninjured proximal-biased kidney organoids. FIG. 12A shows a UMAP reduction colored by sample (injury status: injured vs. uninjured). FIG. 12B shows a UMAP reduction colored by cluster with marker gene annotations. FIG. 12C shows feature plots for HNF4A, HAVCR1, and SOX9, with proximal cells magnified, demonstrating injury-specific expression patterns. FIG. 12D shows a UMAP plot magnifying proximal cells, colored by either cluster or sample. Cell counts within each sample subset are shown. FIG. 12E shows feature plots displaying injury-enriched genes (top row), uninjured-enriched genes (second row), stress-response genes, and pathway upregulation. FIG. 12F shows a percent contribution bar graph for all genes shown in (e), colored by sample type (uninjured or injured). FIG. 12G shows a UMAP reduction of single-cell RNA-sequencing data acquired from the Kidney Precision Medicine Project (KPMP) showing a zoomed-in view of the proximal tubule (PT) cells with three, progressively worsening disease states: Healthy Reference, Acute Kidney Injury (AKI), and Chronic Kidney Disease (CKD). The plot highlights the clustering of proximal tubule cells, including epithelial cells of adaptive/maladaptive/repairing (aPT), degenerative (dPT), proximal tubule segments 1 and 2, and proximal tubule segment 3. FIG. 12H shows feature plots of select injury-enriched, stress-response, and pathway genes. Top three rows display KPMP data, showing progressively worsening disease states from healthy reference to AKI and CKD. Bottom two rows show proximal cells from the PB organoid model, split by sample type, with uninjured cells on the top and double-dose cisplatin-treated cells on the bottom.

FIGS. 13A-13B depict comparative morphological and transcriptomic analyses of proximal nephron formation in vivo and in iPSC-derived kidney organoids. FIGS. 13A-13B show whole-mount immunofluorescent stains of day 10 kidney organoids. Boxed regions are magnified and split into individual channels. Scale bars: 10 microns.

FIGS. 14A-14C depict comparative transcriptomics and analyses of in vivo and early in vitro-forming nephrons. FIGS. 14G-14I show whole-mount immunofluorescent stains of day 10 kidney organoids derived from a different human iPSC line than shown in FIGS. 13A-13B. Boxed region in FIG. 14G is magnified with Z-stack images through the organoid nephron. FIG. 14H is split into individual channels. Proteins stained for in FIGS. 14G-14H match to FIGS. 13C-13D. Scale bars: 10 microns.

FIGS. 15A-15N depict that PI3K inhibitor-treated kidney organoid nephrons exhibit a shift toward early tubular nephron cell fates. FIG. 15A shows UMAP of single-cell RNA-sequencing from day 10 (pre-treatment), day 12 control, and day 12 PI3K inhibitor (LY294002)-treated kidney organoid nephrons. Cells are colored by sample with marker gene annotations. FIG. 15B shows a heatmap of Z-scores for hierarchically clustered differentially expressed genes across samples. Representative genes are listed, with a Z-score legend. FIG. 15C shows feature plots of genes enriched in each sample group from the differentially expressed gene list. FIG. 15D shows feature plots of selected genes for each group based on sample characteristics. For FIGS. 15C-15D, human nephron single-cell detection (FIG. 2A) is shown on the left, organoid plot on the right. FIG. 15E shows UMAP of single-cell RNA-sequencing from day 12 control and day 12 LY294002-treated kidney organoid nephrons. Cells match FIG. 15A but exclude day 10 sample. Cells are colored by sample with marker gene annotations. FIG. 15F shows a heatmap of log₂ fold change (log₂FC) for differentially expressed genes, calculated as the ratio of LY294002-treated to control aggregate expression. Representative genes are listed with a log₂FC legend. FIG. 15G shows feature plots of genes enriched in each sample from the differentially expressed gene list. Human nephron single-cell (FIG. 2A) detection is on the left, organoid plot on the right. h Split violin plots of genes from the differentially expressed gene list. Top two rows show genes enriched in day 12 control cells; bottom two rows show genes enriched in day 12 LY294002-treated cells. Plot colors match sample colors from FIG. 15E. FIG. 15I shows gene ontology terms for the top 50 differentially expressed genes per sample. Top graph: day 12 control nephron cells; bottom graph: LY294002-treated nephron cells. FIGS. 15J-15L shows whole-mount immunofluorescent stains of day 12 control and LY294002-treated kidney organoids. Boxed regions are magnified. Inset in FIG. 15J shows absence of detectable HNF4A protein in day 12 samples. Scale bars: 10 microns. FIG. 15M shows whole-mount immunofluorescent stain of day 12 control and LY294002-treated kidney organoids. Insets highlight JAG1 and HNF1B protein detection. Scale bars: 500 microns. FIG. 15N shows quantification of JAG1⁺ and HNF1B⁺ nephron size (μm²) and HNF1B⁺ intensity (RFU) for n=3 day 12 organoids each, across positive segments. SEM error bars shown. Statistical significance determined by Student's t-test.

FIGS. 16A-16G depict temporal analysis of the Notch pathway during proximal biasing of kidney organoids. FIG. 16A shows whole-mount immunofluorescent stain of organoid nephrons on day 12 following 48 hour culture in control conditions (untreated), LY294002-treated, DAPT-treated, and LY294002⁺ DAPT-treated. Boxed regions highlight clusters of forming organoid nephrons, with split channels. Scale bars: 10 microns. FIG. 16B shows a timeline of inhibitor treatments during differentiation for organoids in FIG. 16A. FIG. 16C shows whole-mount immunofluorescent stains of days 13 and 14 control and PI3K inhibitor-treated (LY294002 from days 10-12, proximal-biased, PB) organoids. Scale bars: 500 microns. FIG. 16D shows whole-mount immunofluorescent stain of control and proximal-biased day 14 organoids with boxed regions magnifying HNF4A detection. Scale bars: 500 microns. FIG. 16E shows live imaging of HNF4A-YFP reporter kidney organoids for control and proximal-biased conditions cultured with or without DAFT for 48 hours post-PI3K inhibitor treatment. Differentiation days 14, 18, and 21 are shown. Scale bars: 500 microns. Day 21 immunofluorescent images included, with boxed regions magnified and JAG1 and HNF1B channels split. Scale bars: 100 microns. FIG. 16F shows a timeline of inhibitor treatments during differentiation for organoids in FIG. 16E. FIG. 16G shows quantification of the number of HNF4A⁺ organoid nephrons for each sample in FIG. 16E on day 21 from n=2 whole kidney organoids. SEM error bars are shown. Statistical significance is determined using Student's t test.

FIGS. 17A-17N depict that kidney organoid nephrons shift toward proximal-biased cell fates following early PI3K inhibitor treatment. FIG. 17A shows UMAP of single-cell RNA-seq from day 14 control and PI3K inhibitor-treated (LY294002 from days 10-12, proximal-biased, PB) kidney organoid nephrons. Cells colored by sample with marker gene annotations. FIG. 17B shows log₂ fold change (log₂FC) heatmap for differentially expressed genes, calculated as the ratio of proximal-biased to control aggregate expression. Representative genes listed with a legend. FIG. 17C shows feature plots of genes enriched in each sample from the differentially expressed gene list. FIG. 17D shows split violin plots of differentially expressed genes. Top two rows: genes enriched in day 14 control cells; bottom two rows: genes enriched in day 14 proximal-biased cells. Plot colors match sample colors from FIG. 17A. FIG. 17E shows feature plots of selected genes for each group based on sample characteristics. For FIG. 17C and FIG. 17E, human nephron single-cell (FIG. 2A) detection is on the left, organoid plot on the right. FIG. 17F shows whole-mount immunofluorescent stains of day 14 control and proximal-biased kidney organoids. Boxed regions split and magnified. White arrowheads mark early HNF4A⁺ nephron regions, yellow arrowheads indicate autofluorescence. Scale bars: 10 microns. FIG. 17G shows bulk RNA-sequencing (TPM) of select genes on days 10, 12, 14, and 18 from n=2 whole organoids. SEM error bars shown. FIGS. 17H-17I show FACS of day 18 kidney organoids from an HNF4A-YFP iPSC reporter line for control (FIG. 17H) and proximal-biased (FIG. 17I) conditions. Brightfield and YFP channels are shown as merged and split panels. FACS plots in duplicates with YFP⁺ percentages in bottom right. Scale bars: 500 microns. FIG. 17J shows immunofluorescent stains of week 16.1 human kidneys during HNF4A⁺, HNF4G⁺ proximal tubule elongation. White arrowheads mark endothelial autofluorescence. Scale bars: 10 microns. FIG. 17K shows an immunofluorescent stain of control and proximal-biased day 18 organoids. Boxed regions magnify HNF4A detection. Scale bars: 200 microns. FIG. 17L shows total HNF4A⁺ nephrons in n=4 whole day 18 organoids. SEM error bars shown. Statistical significance by Student's t-test. FIG. 17M shows average area (μm²) of HNF4A⁺ nephrons from n=4 whole day 18 organoids, across positive segments. SEM error bars shown. Statistical significance by Student's t-test. FIG. 17N shows sum total area (μm²) of HNF4A⁺ nephron segments from n=3 whole day 18 organoids.

FIGS. 18A-18F depict HNF4A and LTL co-detection in control and proximal-biased iPSC-derived kidney organoids, and proximal biasing of iNPC-derived organoids. FIG. 18A shows whole-mount immunofluorescent analysis of control and proximal-biased kidney organoids on differentiation day 18. Split channels are shown, and boxed region is magnified in the bottom row. Scale bars: 100 microns. FIGS. 18B-18C show timelines depicting differentiation days and 48 hour LY294002 treatment proximal biasing) for human iPSC-derived (FIG. 18B) and human induced nephron progenitor cell (iNPC)-derived (FIG. 18C) kidney organoids. FIGS. 18D-18F show whole-mount immunofluorescent stains of control and proximal-biased iNPC-derived kidney organoids. Boxed regions are magnified, and split channels are shown. Scale bars: 10 microns.

FIGS. 19A-19C depict analysis of modifying FGF9 signaling during the proximal biasing stage of kidney organoid differentiation. FIG. 19A shows live imaging of kidney organoids cultured with varying concentrations of recombinant human FGF9 protein between days 10 and 12 of kidney organoid differentiation. Brightfield images for differentiation days 10, 12, 14, and 21 are shown. Day 21 immunofluorescent images are included. Bottom two rows represent the control and proximal-biased (PB) conditions (200 ng/mL FGF9±10 μM LY294002). Scale bars: 500 microns. FIG. 19B shows a timeline of modifying FGF9 levels with or without LY294002 during differentiation for organoids in FIG. 19A. FIG. 19C shows quantification of the number of HNF4A⁺ organoid nephrons for each sample in FIG. 19A on day 21 from n=2 whole kidney organoids. SEM error bars are shown. Statistical significance is determined using Student's t test.

FIGS. 20A-20L depict single-cell transcriptomic and morphological analyses of nephron segment development in proximal-biased kidney organoids across differentiation stages. FIG. 20A shows a schematic of single-cell RNA-sequencing timepoints with UMAP reduction of kidney organoid nephrogenic cells, colored by samples. FIG. 20B shows UMAP reduction of nephrogenic cell clusters in kidney organoids, colored by clusters, with identifying marker gene annotations. UMAPs split by sample type of origin (untreated day 10; control days 12, 14, and 18; or proximal-biased (PB) days 12, 14, and 18) are shown at the top. FIG. 20C shows feature plots for select nephron marker genes with insets separating control from proximal-biased cells, and scaled percent quantification of total cells expressing the gene. FIGS. 20D-20E shows whole-mount immunofluorescent stains of control (FIG. 20D) and proximal-biased (FIG. 20E) kidney organoid nephrons from differentiation days 11, 12, 14, 15, and 18 with the bottom row highlighting HNF4A protein detection. Scale bars: 10 microns. FIGS. 20F-20G depict a differentiation model for control kidney organoids (FIG. 20F) based on FIG. 20D, and for proximal-biased kidney organoids (FIG. 20G) derived from FIG. 20E. FIG. 20H shows time-lapse video captures of kidney organoids differentiated from a human HNF4A-YFP iPSC reporter line from differentiation days 15-18, showing the emergence and progression of HNF4A-YFP detection in control and proximal-biased conditions. Brightfield and YFP channels are merged. Scale bars: 200 microns. FIG. 20I shows whole-mount immunofluorescent stains of control and proximal-biased kidney organoids from differentiation days 14 and 21. Boxed regions are magnified. Scale bars: 10 microns. FIG. 20J shows whole-mount immunofluorescent stains of control and proximal-biased kidney organoids from differentiation days 14, 18, and 21. Boxed regions are magnified. Scale bars: 10 microns. FIG. 20K shows quantification of fold change in the ratio of proximal-biased to control organoid nephrons positive for indicated proteins over differentiation days. Data are from n=3 independent organoids (15×), including those shown in FIGS. 20I-20J, with line colors matching protein labels. SEM error bars shown. FIG. 20L shows quantification of the average segment length (μm) for indicated proteins in day 18 control and proximal-biased kidney organoids. Data are from n=4 independent organoids (15×), including those shown in FIGS. 21C-21D. SEM error bars are shown. Statistical significance is determined using Student's t test.

FIGS. 21A-21D depict whole-mount immunofluorescent analyses of proximal and distal marker detection in differentiation day 18 control and proximal-biased kidney organoids. FIG. 21A shows quantification of central and peripheral HNF4A⁺ and POU3F3⁺ organoid nephrons for control or proximal-biased conditions. The yellow line represents the organoid diameter, with the yellow circle marking the center of the organoid, and its diameter representing the radius of the whole organoid. Blue counts denote peripheral organoid nephrons, while red counts indicate central organoid nephrons. Organoids shown for this panel are the same that were profiled from FIG. 17K. Scale bars: 200 microns. FIG. 21B shows the percent contribution quantifications derived from FIG. 21A. Bar graphs quantify structures out of the total number of central or peripheral structures as either HNF4A⁺ or POU3F3⁺. Data are averaged from n=3 replicate kidney organoids. FIGS. 21C-21D show whole-mount immunofluorescent stains of control and proximal-biased kidney organoids from differentiation day 18. Boxed regions are magnified. Images shown include those used for quantifying the length of nephron segments expressing the proteins analyzed in FIG. 20L. Scale bars: 10 microns.

FIGS. 22A-22K depict single-cell transcriptomics-driven comparison of proximal-biased kidney organoids and proximal tubule-enhanced kidney organoids. FIG. 22A shows UMAP reduction of single-nucleus RNA-sequencing of differentiation day 21 proximal-biased and day 27 proximal-biased (PB) kidney organoids. Cells are colored by cluster, with marker gene annotations. UMAPs split by sample timepoint of origin are shown at the top. FIG. 22B shows a dot plot with identifying genes for each cluster. FIG. 22C shows feature plots for select genes, showing a matching comparison to FIG. 2C and FIG. 2F. FIG. 22D shows UMAP reduction of the nephrogenic subset of day 21 proximal-biased and day 27 proximal-biased kidney organoid single-nucleus RNA-sequencing from FIG. 22A. Cells are colored by sample timepoint of origin, with marker gene annotations. FIG. 22E shows feature plots of select proximal nephron organic cation, organic anion, solute, and drug transporters. Detection of the gene in human nephron single-cell RNA-sequencing (FIG. 2A) data is shown on the left, with the organoid plot on the right. In vivo proximal tubule segment specificity is listed. FIG. 22F shows UMAP reduction of single-cell RNA-sequencing of differentiation day 13⁺14 (27) proximal tubule-enhanced kidney organoids, with marker gene annotations. FIG. 22G shows a dot plot with identifying genes for each cluster. FIG. 22H shows feature plots for select genes, showing a matching comparison to FIG. 2C and FIG. 2F. FIG. 22I shows a diagram of differentiation and sampling timelines for day 21 proximal-biased, day 27 proximal-biased, and day 13⁺14 (27) proximal tubule-enhanced kidney organoids. FIG. 22J shows a schematic of merging single-cell RNA-sequencing datasets: day 21 proximal-biased and day 27 proximal-biased kidney organoids (FIG. 22A) were integrated with differentiation day 13⁺14 (27) proximal tubule-enhanced kidney organoids in FIG. 22F. UMAP reduction of resulting integration is shown, with cells colored by cluster. Split UMAP reductions showing sample of origin are shown on the right. FIG. 22K shows feature plots of select nephron, proximal tubule, and off-target genes, split by sample of origin: proximal-biased cells (left plot) or proximal tubule-enhanced cells (right plot).

FIGS. 23A-23E depict single-cell transcriptomics-driven comparison of proximal-biased and proximal tubule-enhanced kidney organoids, and functional profiling of proximal-biased organoids. FIG. 23A is a diagram of differentiation and sampling timelines for day 21 proximal-biased (PB), day 27 proximal-biased, and day 13⁺14 (27) proximal tubule-enhanced (PT-E) kidney organoids. Sample legend corresponding to violin plots in FIGS. 23B-23C is shown at the bottom. FIG. 23B shows violin plots of representative early proximal tubule genes (expressed in human clusters 21, 24, and 20; FIG. 2A), split by dataset of origin, showing gene expression patterns among expressing cells. FIG. 23C shows violin plots of representative late proximal tubule genes (not expressed in human cluster 21, expressed in clusters 24 and 20; FIG. 2A), split by dataset of origin, showing gene expression patterns among expressing cells. FIG. 23D shows live imaging of control and proximal-biased HNF4A-YFP kidney organoids on differentiation day 21 co-cultured with TRITC-albumin and Alexa 647-conjugated LRP2 antibody. Split channels are shown. In TRITC-albumin panel, yellow arrowheads indicate autofluorescence, while white arrowheads indicate true signal. Scale bars: 100 microns. FIG. 23E shows quantification of HNF4A-YFP⁺ segments with co-detection of 647-LRP2 and TRITC-albumin within the YFP⁺ segment. Counts are averaged from n=3 separate images per organoid across n=3 independent organoids (n=9 images captured at 112× widefield). SEM error bars are shown. Statistical significance determined by Student's t-test.

FIGS. 24A-24N depict functional characterization and injury response in proximal-biased kidney organoids. FIG. 24A shows live imaging of control and proximal-biased HNF4A-YFP kidney organoids on day 21 co-cultured with Alexa 647-Dextran and Alexa 594-LRP2 antibody. Split channels shown. Arrowheads indicate autofluorescence (yellow) and true signal (white). Scale bars: 100 microns. FIG. 24B shows quantification of HNF4A-YFP⁺ segments co-detected with 594-LRP2 and 647-Dextran. Averaged from 3 images/organoid, n=3 organoids. SEM error bars shown. Statistical significance by Student's t-test. FIG. 24C shows whole-mount immunofluorescence of uninjured and cisplatin-injured kidney organoids on day 21, with HNF4A⁺ nephron regions magnified. Scale bars: 10 microns. FIG. 24D shows a timeline of kidney organoid injury strategy. FIG. 24E shows quantification of HAVCR1⁺ signal in control and proximal-biased nephrons on day 21 post-injury (n=3). SEM error bars shown. Statistical significance by Student's t-test. FIG. 24F shows quantification of HNF4A⁺ nephron segments with HAVCR1 expression from FIG. 24C. Data from 4 images/organoid (20×), n=2 replicates. SEM error bars shown. Statistical significance by Student's t-test. FIG. 24G shows whole-mount immunofluorescence of uninjured and cisplatin-injured proximal-biased kidney organoid nephrons on day 21. Dashed white line marks uninterrupted tubule lumen. Scale bars: 10 microns. H Quantification of individual continuous lumen lengths from FIG. 24G, n=4 organoids per condition. SEM error bars shown. Statistical significance by Student's t-test. FIG. 24I shows immunofluorescence of uninjured and cisplatin-injured proximal-biased kidney organoid nephrons on day 21. Yellow arrowheads: HNF4A⁺ tubule cells with low HNF4A and high γH2AX. White arrowheads: cells with high HNF4A and low γH2AX. Split channels for HNF4A and γH2AX shown. Scale bars: 10 microns. FIG. 24J shows immunofluorescence of uninjured and cisplatin-injured kidney organoid nephrons on day 21. White arrowheads: SOX9⁺ interstitial cells. Scale bars: 10 μm. k Quantification of HNF4A and SOX9 intensities from FIG. 24J. Intensities from individual cells in duplicates, min-max normalized. FIG. 24L shows a timeline of kidney organoid single-injury strategy for organoids in FIG. 24M. FIG. 24M shows immunofluorescence of uninjured and cisplatin-injured proximal-biased kidney organoid nephrons on days 19, 21, and 25. Arrowheads indicate cells with high HNF4A and low SOX9 (white), and low HNF4A and high SOX9 (yellow). Scale bars: 10 microns. FIG. 24N is a schematic model of single-injury data, depicting gradual SOX9 activation, collapsing lumens, and loss of HNF4A in organoid nephrons.

### DETAILED DESCRIPTION

It is appreciated that certain features of the disclosure, which are, for clarity, described in the context of separate aspects, can also be provided in combination with a single aspect.

Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

### Definitions

In this specification and in the claims that follow, reference will be made to a number of terms, which shall be defined to have the following meanings:

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a composition”, or “a cancer”, includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a monomer refers to an amount that is sufficient to achieve the desired improvement in the property modulated by the formulation component, e.g. desired antioxidant release rate or viscoelasticity. The specific level in terms of wt % in a composition required as an effective amount will depend upon a variety of factors including the amount and type of monomer, amount and type of polymer, e.g., acrylamide, amount of antioxidant, and desired release kinetics.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed drug delivery composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as an ophthalmological disorder. The effect can be therapeutic in terms of a partial or complete cure of a disease, condition, symptom or adverse effect attributed to the disease, disorder, or condition. The term “treatment” as used herein can include any treatment of ophthalmological disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

### Proximal-Biased Kidney Organoids

In an aspect, provided is a proximal-biased kidney organoid, including a kidney organoid exposed to a PI3 kinase (PI3K) inhibitor and including proximal tubule cells, wherein incubation with the PI3K inhibitor can provide a proximal bias in the kidney organoid. As used herein, the term “proximal bias” or “proximal-biased” refers to kidney organoids in which the majority of tubule cells resemble or function like tubule cells found in a proximal nephron (i.e., proximal tubule cells). For example, a “proximal-biased” kidney organoid can sequentially activate proximal nephron transcription factors and function-imparting proximal tubule genes.

In some aspects, the PI3K inhibitor can include LY294002, GDC-0941, or any combination thereof.

In some aspects, the kidney organoid can be exposed to the PI3K inhibitor for at least about 12 hours (e.g., at least about 16 hours, at least about 20 hours, at least about 1 day, at least about 1.5 days, at least about 2 days, at least about 2.5 days, at least about 3 days, at least about 3.5 days, at least about 4 days, at least about 4.5 days, at least about 5 days). In some aspects, the kidney organoid can be exposed to the PI3K inhibitor for up to about 5 days (e.g., up to about 4.5 days, up to about 4 days, up to about 3.5 days, up to about 3 days, up to about 2.5 days, up to about 2 days, up to about 1.5 days, up to about 1 day, up to about 20 hours, up to about 16 hours, up to about 12 hours). In some aspects, the kidney organoid can be exposed to the PI3K inhibitor for about 12 hours, about 16 hours, about 20 hours, about 1 day, about 1.5 days, about 2 days, about 2.5 days, about 3 days, about 3.5 days, about 4 days, about 4.5 days, or about 5 days.

It is considered that the kidney organoid can be exposed to the PI3K inhibitor for a duration of time ranging from any of the minimum values described above to any of the maximum values described above. For example, in some aspects, the kidney organoid can be exposed to the PI3K inhibitor for from about 12 hours to about 5 days (e.g., from about 16 hours to about 4.5 days, from about 20 hours to about 4 days, from about 1 day to about 3.5 days, from about 1.5 days to about 3 days, from about 2 days to about 2.5 days, from about 12 hours to about 2.5 days, from about 16 hours to about 2 days, from about 20 hours to about 1.5 days, from about 2 days to about 5 days, from about 2.5 days to about 4.5 days, from about 3 days to about 4 days).

In some aspects, the kidney organoid can be exposed to the PI3K inhibitor continuously (i.e., excluding activities related to maintaining or monitoring cell culture, such as changing cell culture media, passaging, imaging, etc.). For example, in some such aspects, the kidney organoid can be exposed to the PI3K inhibitor continuously for from about 12 hours to about 5 days (as described above).

In other aspects, the kidney organoid can be exposed to the PI3K inhibitor intermittently (e.g., from about 1 hour to about 8 hours per day for from about 1 day to about 7 days). For example, in some such aspects, the kidney organoid can be intermittently exposed to the PI3K inhibitor for from at least about 1 hour per day (e.g., at least about 2 hours per day, at least about 3 hours per day, at least about 4 hours per day, at least about 5 hours per day, at least about 6 hours per day, at least 7 hours per day, at least about 8 hours per day). In some such aspects, the kidney organoid can be intermittently exposed to the PI3K inhibitor for up to about 8 hours per day (e.g., up to about 7 hours per day, up to about 6 hours per day, up to about 5 hours per day, up to about 4 hours per day, up to about 3 hours per day, up to about 2 hours per day, up to about 1 hour per day).

It is considered that the kidney organoid can be intermittently exposed to the PI3K inhibitor for a duration ranging from any of the minimum values described above to any of the maximum values described above. For example, in some such aspects, the kidney organoid can be intermittently exposed to the PI3K inhibitor for from about 1 hour to about 8 hours per day (e.g., from about 2 hours to about 7 hours per day, from about 3 hours to about 6 hours per day, from about 4 hours to about 5 hours per day, from about 1 hour to about 5 hours per day, from about 2 hours to about 4 hours per day, from about 4 hours to about 8 hours per day, from about 5 hours to about 7 hours per day).

In some such aspects, the kidney organoid can be intermittently exposed to the PI3K inhibitor over a span of at least about 1 day (e.g., at least about 2 days, at least about 3 days, at least about 4 days, at least about 5 days, at least about 6 days, at least about 7 days). In some such aspects, the kidney organoid can be intermittently exposed to the PI3K inhibitor over a span of up to about 7 days (e.g., up to about 6 days, up to about 5 days, up to about 4 days, up to about 3 days, up to about 2 days, up to about 1 day).

It is considered that the kidney organoid can be intermittently exposed to the PI3K inhibitor over a span ranging from any of the minimum values described above to any of the maximum values described above. For example, in some such aspects, the kidney organoid can be intermittently exposed to the PI3K inhibitor over a span of from about 1 day to about 7 days (e.g., from about 2 days to about 6 days, from about 3 days to about 5 days, from about 1 day to about 4 days, from about 2 days to about 3 days, from about 4 days to about 7 days, from about 5 days to about 6 days).

In some such aspects, the kidney organoid can be intermittently exposed to the PI3K inhibitor for from about 12 hours to about 5 days (as described above) in total. In other such aspects, the kidney organoid can be intermittently exposed to the PI3K inhibitor for about 12 hours or less (e.g., about 10 hours or less, about 8 hours or less, about 6 hours or less, about 4 hours or less, about 3 hours or less, about 2 hours or less, about 1 hour or less) in total.

In yet other aspects, the kidney organoid can be exposed to the PI3K inhibitor using a combination of continuous exposure and intermittent exposure. In some such aspects, the kidney organoid can be exposed to the PI3K inhibitor for from about 12 hours to about 5 days (as described above) in total.

In some aspects, the kidney organoid can be exposed to the PI3K inhibitor at a concentration of at least about 1 μM (e.g., at least about 2 μM, at least about 3 μM, at least about 4 μM, at least about 5 μM, at least about 10 μM, at least about 15 μM, at least about 20 μM, at least about 25 μM, at least about 30 μM, at least about 35 μM, at least about 40 μM, at least about 45 μM, at least about 50 μM, at least about 55 μM, at least about 60 μM, at least about 65 μM, at least about 70 μM, at least about 75 μM at least about 80 μM, at least about 85 μM, at least about 90 μM, at least about 95 μM, at least about 100 μM). In some aspects, the kidney organoid can be exposed to the PI3K inhibitor at a concentration of up to about 100 μM (e.g., up to about 95 μM, up to about 90 μM, up to about 85 μM, up to about 80 μM, up to about 75 μM, up to about 70 μM, up to about 65 μM, up to about 60 μM, up to about 55 μM, up to about 50 μM, up to about 45 μM, up to about 40 μM, up to about 35 μM, up to about 30 μM, up to about 25 μM, up to about 20 μM, up to about 15 μM, up to about 10 μM, up to about 5 μM, up to about 4 μM, up to about 3 μM, up to about 2 μM, up to about 1 μM). In some aspects, the kidney organoid can be exposed to the PI3K inhibitor at a concentration of about 1 μM, about 2 μM, about 3 μM, about 4 μM, about 5 μM, about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, or about 100 μM.

It is considered that the kidney organoid can be exposed to the PI3K inhibitor at a concentration ranging from any of the minimum values described above. For example, in some aspects, the kidney organoid can be exposed to the PI3K inhibitor at a concentration of from about 1 μM to about 100 μM (e.g., from about 2 μM to about 95 μM, from about 3 μM to about 90 μM, from about 4 μM to about 85 μM, from about 5 μM to about 80 μM, from about 10 μM to about 75 μM, from about 15 μM to about 70 μM, from about 20 μM to about 65 μM, from about 25 μM to about 60 μM, from about 30 μM to about 55 μM, from about 35 μM to about 50 μM, from about 40 μM to about 45 μM, from about 1 μM to about 45 μM, from about 2 μM to about 40 μM, from about 3 μM to about 35 μM, from about 4 μM to about 30 μM, from about 5 μM to about 25 μM, from about 10 μM to about 20 μM, from about 40 μM to about 100 μM, from about 45 μM to about 95 μM, from about 50 μM to about 90 μM, from about 55 μM to about 85 μM, from about 60 μM to about 80 μM, from about 65 μM to about 75 μM).

In some aspects, the kidney organoid can further include HNF4A⁺ cells. In some aspects, the kidney organoid can further include an S-shaped body nephron. In some such aspects, the kidney organoid can further include HNF4A⁺ cells localized in a medial segment of the S-shaped body nephron.

In some aspects, the proximal-biased kidney organoid can include human cells.

In some aspects, the proximal-biased organoid can uptake dextran and/or albumin.

In some aspects, the proximal-biased kidney organoid can demonstrate upregulation of KIM1, HAVCR1, HAVCR2, EGR1, STAT3, and/or SOX9⁺ in response to nephrotoxic injury.

### Methods

In an aspect, provided is a method of producing any of the disclosed proximal-biased kidney organoids, the method comprising incubating a plurality of precursor cells with a cell culture media, a PI3K inhibitor, and at least one compound for inducing kidney cell differentiation.

In some aspects, the PI3K inhibitor can include LY294002, GDC-0941, or any combination thereof.

In some aspects, the at least one compound for inducing kidney cell differentiation can include CHIR99021.

In some aspects, the cell culture media can be TeSR-E6.

In some aspects, the plurality of precursor cells can include induced pluripotent stem cells (iPSCs), mesenchymal stem cells (MSCs), nephron progenitor cells, nephron precursor cells, or any combination thereof. In some aspects, the plurality of precursor cells can be human.

In some aspects, the PI3K inhibitor and the at least one compound for inducing kidney cell differentiation can be introduced (i.e., exposed for the first time) to the plurality of precursor cells simultaneously.

In other aspects, the PI3K inhibitor can be introduced to the plurality of precursor cells after the at least one compound for inducing kidney cell differentiation is introduced to the plurality of precursor cells. In some such aspects, the PI3K inhibitor can be introduced to the plurality of precursor cells at least about 5 days (e.g., at least about 6 days, at least about 7 days, at least about 8 days, at least about 9 days, at least about 10 days, at least about 11 days, at least about 12 days, at least about 13 days, at least about 14 days, at least about 15 days) after the at least one compound for inducing kidney cell differentiation is introduced to the plurality of precursor cells. In some such aspects, the PI3K inhibitor can be introduced to the plurality of precursor cells up to about 15 days (e.g., up to about 14 days, up to about 13 days, up to about 12 days, up to about 11 days, up to about 10 days, up to about 9 days, up to about 8 days, up to about 7 days, up to about 6 days, up to about 5 days) after the at least one compound for inducing kidney cell differentiation is introduced to the plurality of precursor cells. In some such aspects, the PI3K inhibitor can be introduced to the plurality of precursor cells 5 days, 6 days, 7 days, 8 days, 9 days, 10 days, 11 days, 12 days, 13 days, 14 days, or 15 days after the at least one compound for inducing kidney cell differentiation is introduced to the plurality of precursor cells.

It is considered that the PI3K inhibitor can be introduced to the plurality of precursor cells a duration ranging from any of the minimum values described above to any of the maximum values described above after the at least one compound for inducing kidney cell differentiation is introduced to the plurality of precursor cells. For example, in some such aspects, the PI3K inhibitor can be introduced to the plurality of precursor cells from about 5 days to about 15 days (e.g., from about 6 days to about 14 days, from about 7 days to about 13 days, from about 8 days to about 12 days, from about 9 days to about 11 days, from about 5 days to about 10 days, from about 6 days to about 9 days, from about 7 days to about 8 days, from about 10 days to about 15 days, from about 11 days to about 14 days, from about 12 days to about 13 days) after the at least one compound for inducing kidney cell differentiation is introduced to the plurality of precursor cells.

In other such aspects, the PI3K inhibitor can be introduced to the plurality of precursor cells at least about 10 minutes (e.g., at least about 15 minutes, at least about 30 minutes, at least about 45 minutes, at least about 1 hour, at least about 2 hours, at least about 4 hours, at least about 8 hours, at least about 12 hours, at least about 1 day, at least about 2 days, at least about 3 days, at least about 4 days, at least about 5 days) after the at least one compound for inducing kidney cell differentiation is introduced to the plurality of precursor cells. In other such aspects, the PI3K inhibitor can be introduced to the plurality of precursor cells up to about 5 days (e.g., up to about 4 days, up to about 3 days, up to about 2 days, up to about 1 day, up to about 12 hours, up to about 8 hours, up to about 4 hours, up to about 2 hours, up to about 1 hour, up to about 45 minutes, up to about 30 minutes, up to about 15 minutes, up to about 10 minutes) after the at least one compound for inducing kidney cell differentiation is introduced to the plurality of precursor cells.

It is considered that the PI3K inhibitor can be introduced to the plurality of precursor cells a duration ranging from any of the minimum values described above to any of the maximum values described above after the at least one compound for inducing kidney cell differentiation is introduced to the plurality of precursor cells. For example, in some such aspects, the PI3K inhibitor can be introduced to the plurality of precursor cells from about 10 minutes to about 5 days (e.g., from about 15 minutes to about 4 days, from about 30 minutes to about 3 days, from about 45 minutes to about 2 days, from about 1 hour to about 1 day, from about 2 hours to about 12 hours, from about 4 hours to about 8 hours, from about 10 minutes to about 8 hours, from about 15 minutes to about 4 hours, from about 30 minutes to about 2 hours, from about 45 minutes to about 1 hour, from about 4 hours to about 5 days, from about 8 hours to about 4 days, from about 12 hours to about 2 days) after the at least one compound for inducing kidney cell differentiation is introduced to the plurality of precursor cells.

Additionally or alternatively, in yet other aspects, the PI3K inhibitor can be introduced to the plurality of precursor cells after formation of a nephron progenitor cell, after formation of a nephron precursor cells, after formation of a proximal tubule precursor cell, and/or before formation of a lumen. Additionally or alternatively, in yet still other aspects, the PI3K inhibitor can be introduced to the plurality of precursor cells upon or after expression of SIX1, PAX2, PAX8, HNF1B, JAG1, and/or HNF4A by the plurality of precursor cells.

In some aspects, the plurality of precursor cells can be separately incubated with the PI3K inhibitor and with the at least one compound for inducing kidney cell differentiation.

In other aspects, the plurality of precursor cells can be incubated with both the PI3K inhibitor and the at least one compound for inducing kidney cell differentiation for about 1 hour or more (e.g., about 2 hours or more, about 4 hours or more, about 8 hours or more, about 12 hours or more, about 1 day or more, about 2 days or more, about 3 days or more, about 4 days or more, about 5 days or more).

In some aspects, the PI3K inhibitor can be introduced to the plurality of precursor cells after the at least one compound for inducing kidney cell differentiation, and the plurality of precursor cells can be separately incubated with the PI3K inhibitor and with the at least one compound for inducing kidney cell differentiation. In other aspects, the PI3K inhibitor can be introduced to the plurality of precursor cells after the at least one compound for inducing kidney cell differentiation, and the plurality of precursor cells can be incubated with both the PI3K inhibitor and with the at least one compound for inducing kidney cell differentiation for about 1 hour or more (as described above).

In some aspects, the plurality of precursor cells can be incubated with the PI3K inhibitor for at least about 12 hours (e.g., at least about 16 hours, at least about 20 hours, at least about 1 day, at least about 1.5 days, at least about 2 days, at least about 2.5 days, at least about 3 days, at least about 3.5 days, at least about 4 days, at least about 4.5 days, at least about 5 days). In some aspects, the kidney organoid can be exposed to the PI3K inhibitor for up to about 5 days (e.g., up to about 4.5 days, up to about 4 days, up to about 3.5 days, up to about 3 days, up to about 2.5 days, up to about 2 days, up to about 1.5 days, up to about 1 day, up to about 20 hours, up to about 16 hours, up to about 12 hours). In some aspects, the plurality of precursor cells can be incubated with the PI3K inhibitor for about 12 hours, about 16 hours, about 20 hours, about 1 day, about 1.5 days, about 2 days, about 2.5 days, about 3 days, about 3.5 days, about 4 days, about 4.5 days, or about 5 days.

It is considered that the plurality of precursor cells can be incubated with the PI3K inhibitor for a duration of time ranging from any of the minimum values described above to any of the maximum values described above. For example, in some aspects, the plurality of precursor cells can be incubated with the PI3K inhibitor for from about 12 hours to about 5 days (e.g., from about 16 hours to about 4.5 days, from about 20 hours to about 4 days, from about 1 day to about 3.5 days, from about 1.5 days to about 3 days, from about 2 days to about 2.5 days, from about 12 hours to about 2.5 days, from about 16 hours to about 2 days, from about 20 hours to about 1.5 days, from about 2 days to about 5 days, from about 2.5 days to about 4.5 days, from about 3 days to about 4 days).

In some aspects, the plurality of precursor cells can be incubated with the PI3K inhibitor continuously (i.e., excluding activities related to maintaining or monitoring cell culture, such as changing cell culture media, passaging, imaging, etc.). For example, in some such aspects, the plurality of precursor cells can be continuously incubated with the PI3K inhibitor for from about 12 hours to about 5 days (as described above).

In other aspects, the plurality of precursor cells can be incubated with the PI3K inhibitor intermittently (e.g., from about 1 hour to about 8 hours per day for from about 1 day to about 7 days). For example, in some such aspects, the plurality of precursor cells can be intermittently incubated with the PI3K inhibitor for from at least about 1 hour per day (e.g., at least about 2 hours per day, at least about 3 hours per day, at least about 4 hours per day, at least about 5 hours per day, at least about 6 hours per day, at least 7 hours per day, at least about 8 hours per day). In some such aspects, the plurality of precursor cells can be intermittently incubated with the PI3K inhibitor for up to about 8 hours per day (e.g., up to about 7 hours per day, up to about 6 hours per day, up to about 5 hours per day, up to about 4 hours per day, up to about 3 hours per day, up to about 2 hours per day, up to about 1 hour per day).

It is considered that the plurality of precursor cells can be intermittently incubated with the PI3K inhibitor for a duration ranging from any of the minimum values described above to any of the maximum values described above. For example, in some such aspects, the plurality of precursor cells can be intermittently incubated with the PI3K inhibitor for from about 1 hour to about 8 hours per day (e.g., from about 2 hours to about 7 hours per day, from about 3 hours to about 6 hours per day, from about 4 hours to about 5 hours per day, from about 1 hour to about 5 hours per day, from about 2 hours to about 4 hours per day, from about 4 hours to about 8 hours per day, from about 5 hours to about 7 hours per day).

In some such aspects, the plurality of precursor cells can be intermittently incubated with the PI3K inhibitor over a span of at least about 1 day (e.g., at least about 2 days, at least about 3 days, at least about 4 days, at least about 5 days, at least about 6 days, at least about 7 days). In some such aspects, the plurality of precursor cells can be intermittently incubated with the PI3K inhibitor over a span of up to about 7 days (e.g., up to about 6 days, up to about 5 days, up to about 4 days, up to about 3 days, up to about 2 days, up to about 1 day).

It is considered that the plurality of precursor cells can be intermittently incubated with the PI3K inhibitor over a span ranging from any of the minimum values described above to any of the maximum values described above. For example, in some such aspects, the plurality of precursor cells can be intermittently incubated with the PI3K inhibitor over a span of from about 1 day to about 7 days (e.g., from about 2 days to about 6 days, from about 3 days to about 5 days, from about 1 day to about 4 days, from about 2 days to about 3 days, from about 4 days to about 7 days, from about 5 days to about 6 days).

In some such aspects, the plurality of precursor cells can be intermittently incubated with the PI3K inhibitor for from about 12 hours to about 5 days (as described above) in total. In other such aspects, the plurality of precursor cells can be intermittently incubated with the PI3K inhibitor for about 12 hours or less (e.g., about 10 hours or less, about 8 hours or less, about 6 hours or less, about 4 hours or less, about 3 hours or less, about 2 hours or less, about 1 hour or less) in total.

In yet other aspects, the plurality of precursor cells can be incubated with the PI3K inhibitor using a combination of continuous incubation and intermittent incubation. In some such aspects, the plurality of precursor cells can be incubated with the PI3K inhibitor for from about 12 hours to about 5 days (as described above) in total.

In some aspects, the PI3K inhibitor can be present in a concentration of at least about 1 μM (e.g., at least about 2 μM, at least about 3 μM, at least about 4 μM, at least about 5 μM, at least about 10 μM, at least about 15 μM, at least about 20 μM, at least about 25 μM, at least about 30 μM, at least about 35 μM, at least about 40 μM, at least about 45 μM, at least about 50 μM, at least about 55 μM, at least about 60 μM, at least about 65 μM, at least about 70 μM, at least about 75 μM at least about 80 μM, at least about 85 μM, at least about 90 μM, at least about 95 μM, at least about 100 μM). In some aspects, the PI3K inhibitor can be present in a concentration of up to about 100 μM (e.g., up to about 95 μM, up to about 90 μM, up to about 85 μM, up to about 80 μM, up to about 75 μM, up to about 70 μM, up to about 65 μM, up to about 60 μM, up to about 55 μM, up to about 50 μM, up to about 45 μM, up to about 40 μM, up to about 35 μM, up to about 30 μM, up to about 25 μM, up to about 20 μM, up to about 15 μM, up to about 10 μM, up to about 5 μM, up to about 4 μM, up to about 3 μM, up to about 2 μM, up to about 1 μM). In some aspects, the PI3K inhibitor can be present in a concentration of about 1 μM, about 2 μM, about 3 μM, about 4 μM, about 5 μM, about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM, or about 100 μM.

It is considered that the PI3K inhibitor can be present in a concentration ranging from any of the minimum values described above. For example, in some aspects, the PI3K inhibitor can be present in a concentration of from about 1 μM to about 100 μM (e.g., from about 2 μM to about 95 μM, from about 3 μM to about 90 μM, from about 4 μM to about 85 μM, from about 5 μM to about 80 μM, from about 10 μM to about 75 μM, from about 15 μM to about 70 μM, from about 20 μM to about 65 μM, from about 25 μM to about 60 μM, from about 30 μM to about 55 μM, from about 35 μM to about 50 μM, from about 40 μM to about 45 μM, from about 1 μM to about 45 μM, from about 2 μM to about 40 μM, from about 3 μM to about 35 μM, from about 4 μM to about 30 μM, from about 5 μM to about 25 μM, from about 10 μM to about 20 μM, from about 40 μM to about 100 μM, from about 45 μM to about 95 μM, from about 50 μM to about 90 μM, from about 55 μM to about 85 μM, from about 60 μM to about 80 μM, from about 65 μM to about 75 μM).

In some aspects, the plurality of precursor cells can be incubated with at least one additional cell growth and/or differentiation compound. In some aspects, the at least one additional cell growth and/or differentiation compound can include FGF, heparin, or a combination thereof.

In some aspects, the plurality of precursor cells can be incubated with the cell culture media alone for at least about 1 day (e.g., at least about 2 days, at least about 3 days, at least about 4 days, at least about 5 days, at least about 6 days, at least about 7 days, at least about 8 days, at least about 9 days, at least about 10 days). In some aspects, the plurality of precursor cells can be incubated with the cell culture media alone for up to about 10 days (e.g., up to about 9 days, up to about 8 days, up to about 7 days, up to about 6 days, up to about 5 days, up to about 4 days, up to about 3 days, up to about 2 days, up to about 1 day). In some aspects, the plurality of precursor cells can be incubated with the cell culture media alone for about 1 day, about 2 days, about 3 days, about 4 days, about 5 days, about 6 days, about 7 days, about 8 days, about 9 days, or about 10 days.

It is considered that the plurality of precursor cells can be incubated with the cell culture media alone for a duration ranging from any of the minimum values described above to any of the maximum values described above. For example, in some aspects, the plurality of precursor cells can be incubated with the cell culture media alone for from about 1 day to about 10 days (e.g., from about 2 days to about 9 days, from about 3 days to about 8 days, from about 4 days to about 7 days, from about 5 days to about 6 days, from about 1 day to about 6 days, from about 2 days to about 5 days, from about 3 days to about 4 days, from about 5 days to about 10 days, from about 6 days to about 9 days, from about 7 days to about 8 days.

In some aspects, the plurality of precursor cells can be incubated with the cell culture media alone for about 1 day or less (e.g., about 20 hours or less, about 16 hours or less, about 12 hours or less, about 8 hours or less, about 4 hours or less, about 2 hours or less, about 1 hour or less). In some aspects, the plurality of precursor cells can be incubated with the cell culture media alone for 10 days or more (e.g., 12 days or more, 14 days or more, 16 days or more, 18 days or more, 20 days or more).

In some aspects, the plurality of precursor cells can be incubated with the cell culture media alone after the plurality of precursor cells are incubated with the PI3K inhibitor and the at least one compound for inducing kidney cell differentiation. In other aspects, the plurality of precursor cells can be incubated with the cell culture media alone before the plurality of precursor cells are incubated with the PI3K inhibitor and the at least one compound for inducing kidney cell differentiation. In yet other aspects, the plurality of precursor cells can be incubated with the cell culture media alone after the plurality of precursor cells are incubated with the at least one compound for inducing kidney cell differentiation and before the plurality of precursor cells are incubated with the PI3K inhibitor.

In yet still other aspects, the plurality of precursor cells can be incubated with the cell culture media any combination of times (e.g., before the plurality of precursor cells are incubated with the PI3K inhibitor and the at least one compound for inducing kidney cell differentiation, after the plurality of precursor cells are incubated with the at least one compound for inducing kidney cell differentiation and before the plurality of precursor cells are incubated with the PI3K inhibitor, and/or after the plurality of precursor cells are incubated with the PI3K inhibitor and the at least one compound for inducing kidney cell differentiation).

In some such aspects, the plurality of precursor cells can be incubated with the cell culture media for from about 1 day to about 10 days (as described above) in total. In other such aspects, the plurality of precursor cells can be incubated with the cell culture media for from about 1 day to about 10 days (as described above) each time. In yet still other such aspects, the plurality of precursor cells can be incubated with the cell culture media for 1 day or less (as described above) in total. In yet still other such aspects, the plurality of precursor cells can be incubated with the cell culture media for 1 day or less (as described above) each time. In yet still other such aspects, the plurality of precursor cells can be incubated with the cell culture media for 10 days or more (as described above) in total. In yet still other such aspects, the plurality of precursor cells can be incubated with the cell culture media for 10 days or more (as described above) each time.

In some aspects, the plurality of precursor cells can be incubated for at least about 15 days (e.g., at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days) in total. In some aspects, the plurality of precursor cells can be incubated for up to about 30 days (e.g., up to about 29 days, up to about 28 days, up to about 27 days, up to about 26 days, up to about 25 days, up to about 24 days, up to about 23 days, up to about 22 days, up to about 21 days, up to about 20 days, up to about 19 days, up to about 18 days, up to about 17 days, up to about 16 days, up to about 15 days) in total. In some aspects, the plurality of precursor cells can be incubated for about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, or about 30 days in total.

It is considered that the plurality of precursor cells can be incubated for a duration ranging from any of the minimum values described above to any of the maximum values described above in total. For example, in some aspects, the plurality of precursor cells can be incubated for from about 15 days to about 30 days (e.g., from about 16 days to about 29 days, from about 17 days to about 28 days, from about 18 days to about 27 days, from about 19 days to about 26 days, from about 20 days to about 25 days, from about 21 days to about 24 days, from about 22 days to about 23 days, from about 15 days to about 23 days, from about 16 days to about 22 days, from about 17 days to about 21 days, from about 18 days to about 20 days, from about 22 days to about 30 days, from about 23 days to about 29 days, from about 24 days to about 28 days, from about 25 days to about 27 days) in total.

In some aspects, the plurality of precursor cells can be incubated for about 15 days or less (e.g., about 14 days or less, about 13 days or less, about 12 days or less, about 11 days or less, about 10 days or less, about 9 days or less, about 8 days or less, about 7 days or less, about 6 days or less, about 5 days or less). In some aspects, the plurality of precursor cells can be incubated for about 30 days or more (e.g., about 35 days or more, about 40 days or more, about 45 days or more, about 50 days or more).

In another aspect, provided is a method of modeling a kidney disease, the method including: a) providing any of the disclosed proximal-biased kidney organoids; and b) observing the proximal-biased kidney organoid over a period of time.

In some aspects, the proximal-biased kidney organoid can include healthy cells, and step a) can further include physically and/or chemically damaging the proximal-biased kidney organoid. For example, in some such aspects, physically and/or chemically damaging the proximal-biased kidney organoid can include ablation, scraping, exposure to radiation, and/or exposure to chemical agents (e.g., chemotherapy agents, chemical irritants, etc.).

In other aspects, the proximal-biased kidney organoid can include diseased or abnormal cells. In some such aspects, the proximal-biased kidney organoid can include cells consistent with a renal tubule disease or disorder and/or a nephrotic disease or disorder.

In some aspects, the proximal-biased kidney organoid can be derived from cells taken from a patient. In some such aspects, the patient can have a kidney disease or disorder. In other such aspects, the patient can be healthy.

In some aspects, the method can further include administering a therapeutic agent to the proximal-biased kidney organoid. In some such aspects, the therapeutic agent can include a small molecule, a chemotherapy agent, a biologic agent (e.g., a peptide or antibody), a nucleic acid, a therapeutic cell, a therapeutic treatment (e.g., surgery/ablation, radiation, etc.), or any combination thereof.

In some aspects, the proximal-biased kidney organoid can be observed over a period of minutes, hours, days, weeks, or months.

In another aspect, provided is a method of screening for therapeutic agents that modulate injury to proximal tubule cells, the method including: a) providing any of the disclosed proximal-biased kidney organoids; b) administering a therapeutic agent to the proximal-biased kidney organoid; and c) observing the proximal-biased kidney organoid over a period of time.

In some aspects, step a) can further include physically and/or chemically damaging the proximal-biased kidney organoid, and the method can be used to screen for therapeutic agents that treat or improve injury to proximal tubule cells. For example, in some such aspects, physically and/or chemically damaging the proximal-biased kidney organoid can include ablation, scraping, exposure to radiation, and/or exposure to chemical agents (e.g., chemotherapy agents, chemical irritants, etc.).

In other aspects, the proximal-biased kidney organoid can include healthy cells, and the method can be used to screen for therapeutic agents that do not cause injury to proximal tubule cells.

In yet other aspects, the method can further include, after step b) and before step c), physically and/or chemically damaging the proximal-biased kidney organoid, and the method can be used to screen for therapeutic agents that prevent or lessen injury to proximal tubule cells. For example, in some such aspects, physically and/or chemically damaging the proximal-biased kidney organoid can include ablation, scraping, exposure to radiation, and/or exposure to chemical agents (e.g., chemotherapy agents, chemical irritants, etc.).

In some aspects, the therapeutic agent can include a small molecule, a chemotherapy agent, a biologic agent (e.g., a peptide or antibody), a nucleic acid, a therapeutic cell, a therapeutic treatment (e.g., surgery/ablation, radiation, etc.), or any combination thereof.

In some aspects, the proximal-biased kidney organoid can be observed over a period of minutes, hours, days, weeks, or months.

### EXAMPLES

### Example 1: Stepwise Developmental Mimicry Generates Proximal-Biased Kidney Organoids

Directed differentiation protocols coaxing induced pluripotent stem cells (iPSCs) to intermediate mesoderm lineages have led to the development of human kidney-like. These models partially replicate developing kidney cell profiles, but do not form mature proximal tubule cells, and the proximal precursor-like cells that do develop exhibit low expression of genes that normally impart nephron-specific physiologies. While current organoid models have demonstrated upregulation of specific injury markers such as KIM1/HAVCR1 and γH2AX in LTL⁺ cells, the lack of homogenous proximal tubule like cells in organoid limits their utility in studying acute proximal tubular injury and performing proximal nephron-specific drug screens

Studies performed in mice and organoids show that proximal tubule development is dependent on expression of transcription factor Hnf4a/HNF4A, whose protein product is required for the normal expression of roughly 300 proximal tubule solute carriers, protein, ion, and substrate transporters, and other proximal nephron functional genes. Recently, effort has therefore focused on generating HNF4A⁺ proximal tubule precursors as these would serve as building blocks for generating functional proximal tubules. One strategy has enriched for this cell population by expanding the pool of nephron progenitors in early organoids and subsequently allowed their differentiation, but regardless of the protocol, proximal nephron precursors display relatively low expression of HNF4A and HNF4A-dependent genes.

In vivo, proximal precursors emerge in the S-shaped body nephron following a stereotyped and deeply conserved developmental program. In this program, nephron progenitors are gradually recruited from their niche into pretubular aggregates that sequentially undergo epithelial-to-mesenchymal transitions generating epithelial renal vesicles. Complex morphogenetic events form tubular Comma-shaped and thereafter the aforementioned S-shaped body (SSB) nephrons with distal and proximal gene signatures positioned along the emerging distal-to-proximal axial polarity. Transcriptionally distinct HNF4A⁺ proximal tubule precursors develop in narrow 2-3 cell-wide populations within each medial domain of SSBs, in a field of Notch ligand JAG1⁺ cells. Their development is dependent on Notch signaling, as Notch1 and Notch2 loss-of-function mice fail to express transcription factor Hnf1b, which in turn binds to and is necessary for Hnf4a expression. In addition to Notch, the development of the proximal-distal nephron axis requires integrated signaling between several pathways as spatial positions in the nascent nephron are known to be driven by Notch, Wnt, BMP, and PI3K signaling, each tuning the formation of various precursor populations. Nephrons forming in kidney organoids respond to changes in Wnt signaling in a manner conserved with that shown in mouse kidneys, but there is no clear maturation of cells. This raises the possibility to developmentally program organoid nephrons to form proximal tubule precursors, but such strategies have not been identified and are therefore required.

This study addresses this challenge by developing a protocol to expand the HNF4A⁺ proximal nephron precursor population within kidney organoids and in individual nephrons. Organoid cell differentiation was directed along an in vivo-like developmental trajectory proceeding through JAG1⁺/HNF1B⁺ fates and culminating in HNF4A⁺ proximal precursors. Comparative analyses with in vivo development show the organoid proximal precursors mature to resemble HNF4A⁺ cells in the capillary loop stage (CLSN) human nephron with emerging physiologies. Proximal-biased (PB) nephrons display expression of solute carriers and transporters and are capable of selectively transporting albumin and dextran. This study further leverages the prevalence of proximal structures within the PB organoid model to demonstrate significant upregulation of KIM1/HAVCR1 within HNF4A⁺ organoid nephron tubules in response to nephrotoxic injury. The PB nephron model represents a significant step towards recapitulating kidney development and function in organoid systems and provides a direct strategy to study proximal nephrotoxicity and tubulopathies in a robust human assay.

### Materials and Methods

Human kidney samples: Human fetal samples were collected under Institutional Review Board approved protocols (USC-HS-13-0399 and CHLA-14-2211). Following the patient decision for pregnancy termination, informed consent for donation of the products of conception for research purposes was obtained and samples collected without patient identifiers. The person obtaining informed consent was different than the physician performing the pregnancy termination procedure, and the decision to donate tissue did not impact the method of pregnancy termination. Fetal age was determined according to the American College of Obstetrics and Gynecology guidelines. The kidney samples ranged from 14 to 17 weeks of gestation with no sex reported. Intact samples within the kidney capsule were analyzed. Samples were transported on ice at 4° C. in high glucose DMEM (Gibco, 11965-118) supplemented with 10% fetal bovine serum (Genesee Scientific, 25-550) and 25 mM HEPES (Gibco, 15630080).

Human induced pluripotent stem cell lines: An iPSC line (male) was used. An HNF4A-YFP reporter iPSC line (male) was also used. Details on the HNF4A-YFP iPSC line can be found elsewhere.

Mouse kidney explants and culture: Pregnant female Swiss Webster mice were euthanized on day E12.5 of pregnancy via CO₂ following appropriate University of Southern California IACUC approved protocol. Embryos were removed and placed into PBS before dissecting out kidneys for explant culture as described below.

### Kidney Organoid Cultures

iPSC maintenance: Matrigel-coated plate preparation. DMEM (Corning, 10-017-CV) was aliquoted into a 15 mL conical vial, and 120 μL of Matrigel (Corning, 354277) was added to make a 1% Matrigel mix. After thorough mixing, 2 mL of 1% Matrigel was pipetted into each well of a 6-well plate (or 1 mL/well for a 12-well plate). Matrigel plates were then incubated at 37° C./5% CO2 overnight before use.

Biolaminin 521 LN-coated plate preparation: DMEM was aliquoted into a 15 mL conical vial and 300 μL of biolaminin 521 LN (from Biolamina, LN521) was added to make a 5% biolaminin 521 LN mix. After thorough mixing, 0.5 mL of 5% biolaminin 521 LN was pipetted into each well of a 12-well plate. Biolaminin 521 LN plates were then incubated at 4° C. overnight before use.

iPSC expansion and maintenance: iPSCs were thawed in Essential 8 media (Thermo Fisher Scientific, A1517001) on 1% Matrigel-coated plates. Media was initially supplemented with 10 μM Y-27632 (Rho kinase inhibitor from Tocris, 1254) for 24 hours after thawing iPSCs. Media was changed every 24 hours until cells reached 70-80% confluency (3 days). For freezing, when iPSCs reached 70-80% confluency, cells were collected for passage as described below and resuspended into a mix of 50% Essential 8, 40% KnockOut Serum Replacement (Thermo Fisher Scientific, 10828010), and 10% DMSO (Millipore Sigma, D5879). Cells were then aliquoted into 2 mL cryovials at 500,000 cells/vial, gradually frozen at −80° C. overnight, and then transferred to liquid nitrogen storage.

Directed differentiation to generate kidney organoids: Differentiation protocols were developed based on published protocols and adapted in the laboratory. Each biological replicate was generated from a distinct frozen vial of iPSCs. On reaching 70-80% confluency (3 days), iPSCs were rinsed with 1×PBS (Thermo Fisher Scientific, 10010049) and then incubated in TrypLE Select Enzyme (Thermo Fisher Scientific, 12563011) for 6 minutes at 37° C./5% CO₂. The enzymatic reaction was neutralized using a volume of Essential 8 media 2× that of TrypLE. Cells were collected and resuspended in Essential 8 media supplemented with 10 μM Y-27632, with 10,000 cells/well plated onto a 5% biolaminin 521 LN-coated 12-well plate. 6 hours after plating, differentiation was initiated by changing culture media to TeSR-E6 (Stem Cell Technologies, 05946) supplemented with CHIR99021 (Tocris, 4423). Briefly, culture medium was supplemented with CHIR99021 for 5 days (exact concentration and duration are dependent on cell line being used), followed by 2 days with 200 ng/mL FGF-9 (R&D Systems, 273-F9) and 1 μg/mL Heparin (Millipore Sigma, H4784). At day 7, the cells were detached using TrypLE as described above and resuspended in TeSR-E6 supplemented with 10 μM Y-27632. 200,000 cells were seeded into each well of a round-bottom non-adhesive 96-well plate. Organoids were manually transferred to a 0.4 μm pore culture plate (Corning, 3450; Stem Cell Technologies, 100-1026). Organoids received a pulse of TeSR-E6 supplemented with CHIR99021 for 1 hour; media was then switched back to TeSR-E6 supplemented with 200 ng/mL FGF-9 and 1 μg/mL Heparin until day 12. Proximal-biased kidney organoids were cultured in TeSR-E6 supplemented with 200 ng/mL FGF-9, 1 μg/mL Heparin, and 10 μM LY294002 (Tocris, 1130) for 48 hours between differentiation days 10 and 12; untreated control organoids were cultured with 200 ng/mL FGF-9, 1 μg/mL Heparin, and DMSO vehicle. From day 12 onward, organoids were cultured in TeSR-E6 alone.

Small-molecule inhibitor screen and culture optimization: For all small molecule inhibitors used in this study, initial screens were performed on kidney organoids to determine the optimal dosage and duration of culture. To determine optimal dosage of a molecule, kidney organoids were cultured for 48 hours between differentiation days 10 and 12 in TeSR-E6 supplemented with the molecule at varying concentrations. Concentrations tested used insights from prior studies. After an ideal concentration was established, to determine optimal duration of treatment with the small molecule, separate differentiation experiments were set up with the small molecule from differentiation days 10 to 18, as well as differentiation days 12 to 18. For all optimization screens, time-course bright-field imaging was performed, and organoids were collected for whole-mount immunofluorescent analyses on differentiation day 18.

Mouse kidney organ culture: Kidneys were dissected from E12.5 embryos and cultured for 48 hours at 37° C./5% CO₂ in DMEM. Treatment condition culture media was initially supplemented with 20 μM LY294002, 2 μM DAPT (Tocris 2634), or both, before 48 hours of culture, following prior studies. After culture, mouse kidney explants were prepared for immunofluorescent analyses as described below for organoids.

### Characterization of the Organoid System

Kidney organoid cell dissociation: Approximately 6 organoids from multiple independent wells of both untreated and treated (48 hour culture with 10 μM LY294002 between days 10 and 12) conditions were collected at differentiation days 10, 12, 14, and 18 for single-cell RNA-sequencing, from separate differentiation batches. The organoids were dissociated using Accumax (Stem Cell Technologies, 07921) at 37° C./5% CO₂ for 35 minutes and pipetted twenty times with a P-1000 wide-bore pipette tip every 5 minutes. The dissociation was neutralized by using a 2× volume of AutoMACS Running Buffer (Miltenyi Biotec, 130-091-221). Cells were pelleted by centrifugation at 4° C. for 3 minutes at 300×g. After resuspending the pellet in 2 mL of AutoMACS Running Buffer, cell suspensions were strained through 40 μm strainer (VWR, 21008-949), washing the filter with an additional 1 mL AutoMACS. The flow-through was pelleted, again at 300×g in a 4° C. for 3 minutes. Cells were gently resuspended in AutoMACS Running Buffer supplemented with 14 μM DAPI (Thermo Fisher Scientific, D1306) and 5 μM DRAQ5 (Thermo Fisher Scientific, 62-254), and sorted on an ARIA II FACS at a low flow rate. Viable cells (DRAQ5-positive, DAPI-negative) were collected into a 1.7 mL low binding tube (Corning, 3207).

SPLiT-seq single-cell RNA-sequencing data collection and analyses: SPLiT-seq cell processing. Sorted, single, live cells were immediately processed and fixed using Evercode Cell Fixation kit (Parse Biosciences, ECF2001). Fixed cells were then stored at −80° C. until all samples were ready to be processed with the Evercode WT v2 single-cell RNA-sequencing platform (Parse Biosciences, ECW02030). After recovery of barcoded cells, cDNA was cleaned up and amplified by PCR, as per Evercode WT v2 platform protocols. cDNA quality was examined at multiple points on a 4200 TapeStation (Agilent) for yield and quality assessment. Paired-end sequencing was performed on the NovaSeq X Plus PE150 (Novogene).

scRNA-seq analyses: From fastq files, demultiplexing, quality control, alignment to reference genome (hg38 Ensembl 105 annotation), and generation of count tables were done using splitpipe (Parse Biosciences). The Seurat 4.0 package was used for single-cell RNA-sequencing (scRNA-seq) analyses. To filter out low-quality cells, the study kept cells with between 500 and 13000 features, between 100 and 200000 RNA counts, and less than 35% mitochondrial gene content. Sample datasets were integrated using Seurat integration functions across organoid time-points and experimental variables. 50 PCs were used to calculate cell relationships, cluster assignment, and UMAPs. After quality control and sample integration, 56,320 cells remained. Differentially expressed genes of each cluster were identified using the FindAllMarkers function, selectively returning genes with expression in at least 25% of cells within the cluster (min.pct=0.25) and with a minimum fold change of 0.25 (log fc.threshold=0.25), while restricting the output to only positively expressed markers (only.pos=TRUE). Nephrogenic cell-clusters were identified based on expression of nephron lineage and nephron cell-fate markers and interstitial lineages similarly separated by their gene enrichment. Nephron-forming cells were selected by serial clustering and subsetting while monitoring cluster-based quality metrics. Clusters 3, 6, 8, 9, 10, 13, 14, 15, 16, 17, 20, 24, 26, and 27 were subset for nephrogenic lineage examination. scRNA-sequencing data are deposited with GEO accession number GSE264678 and will be made publicly available as of the date of publication.

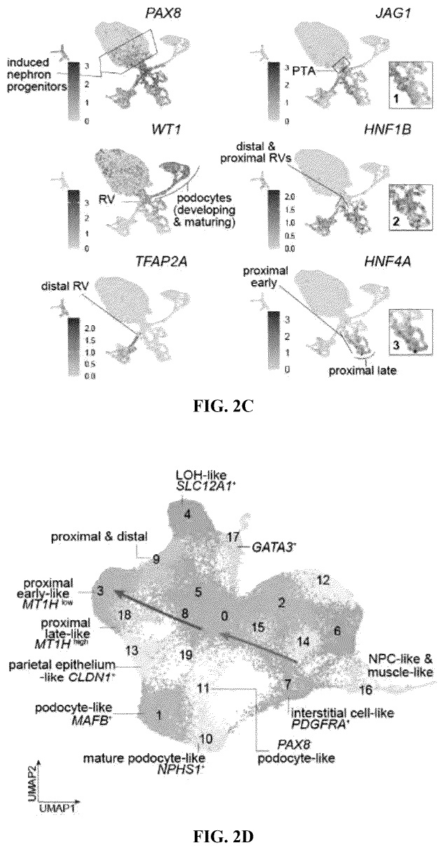

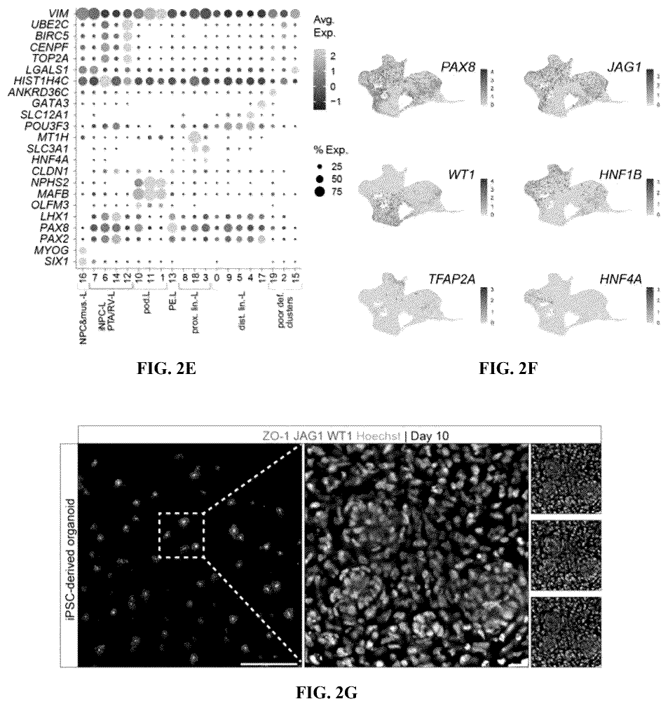

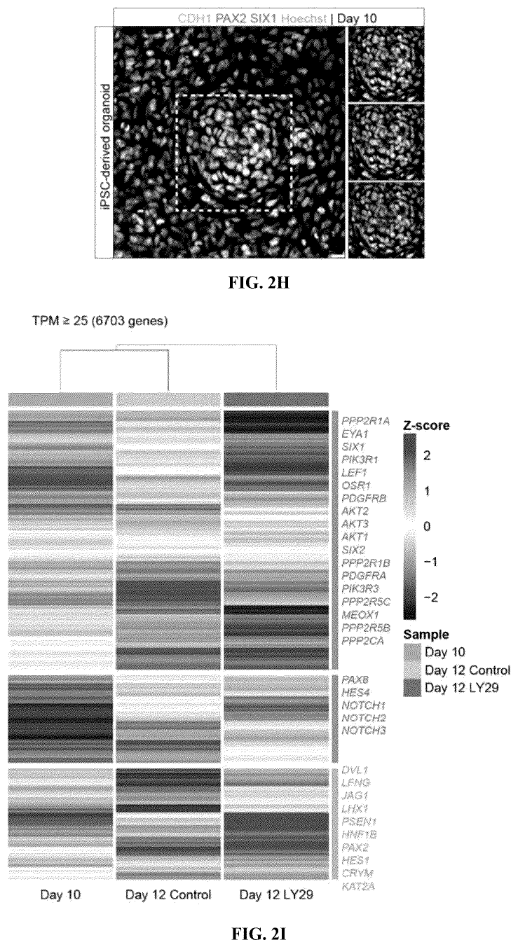

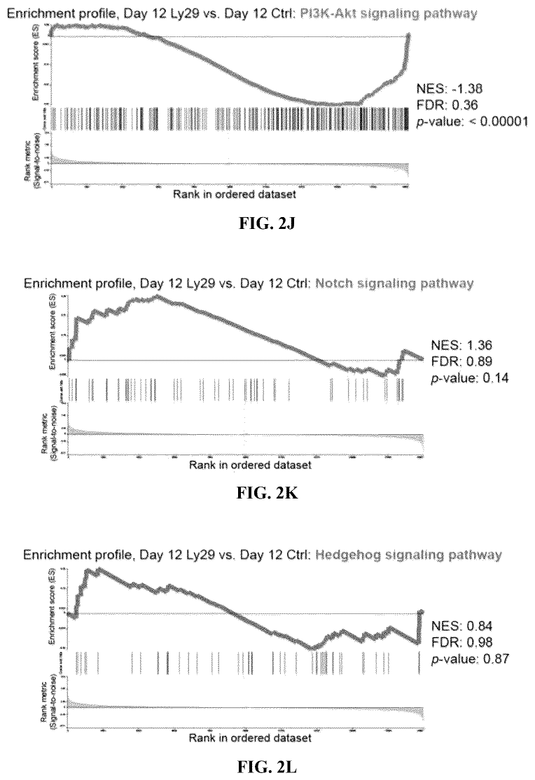

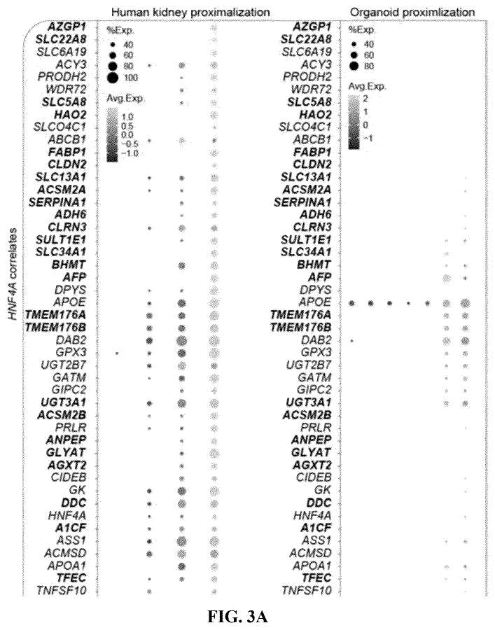

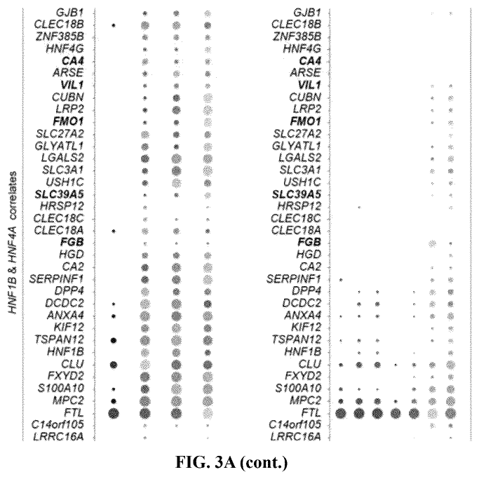

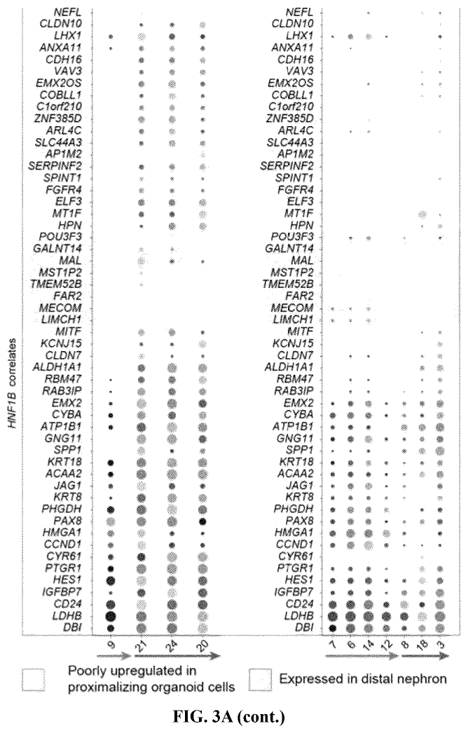

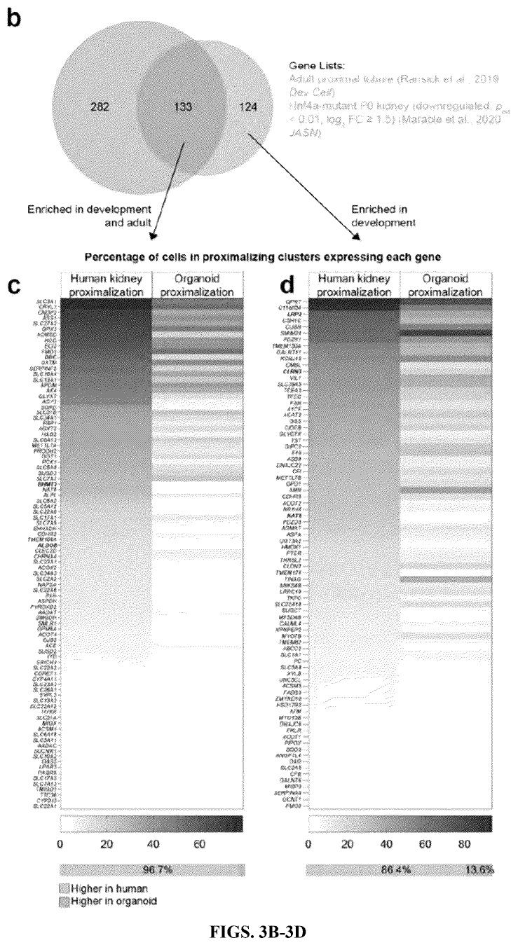

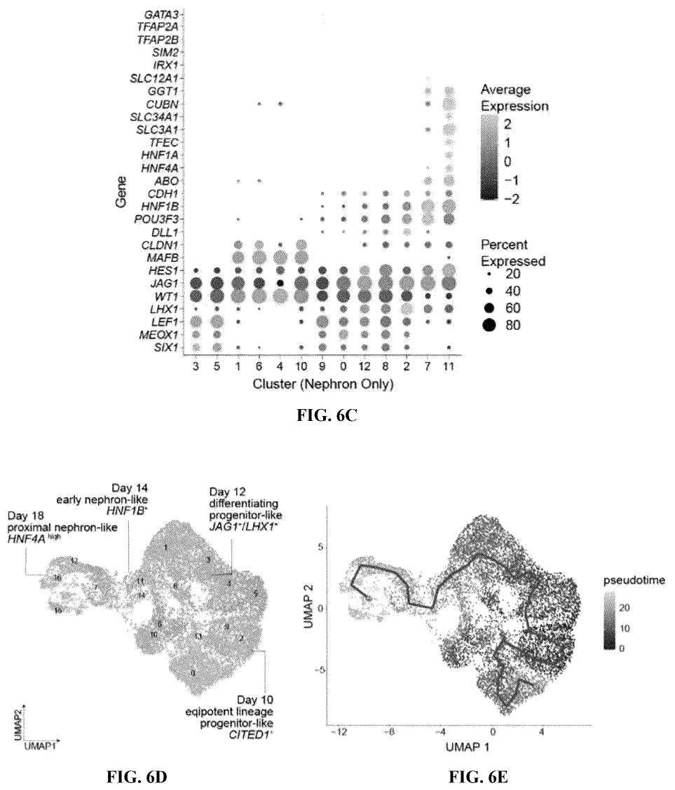

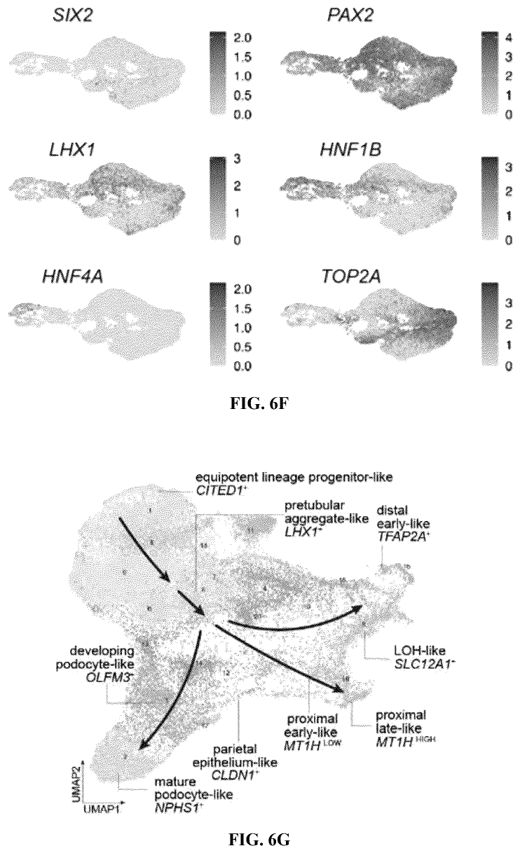

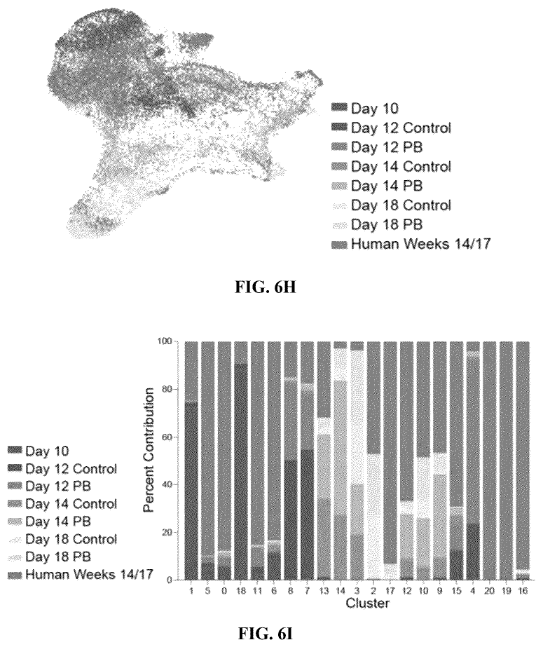

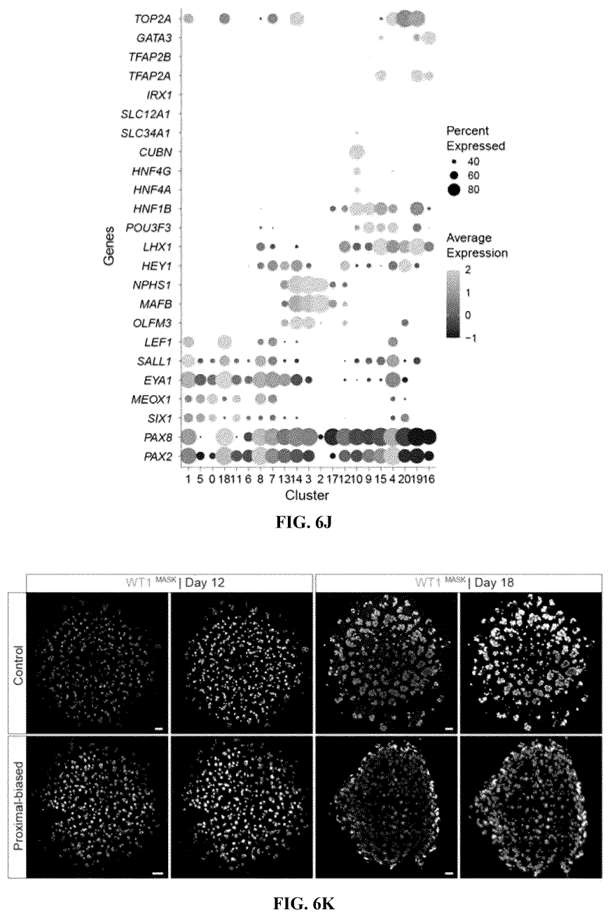

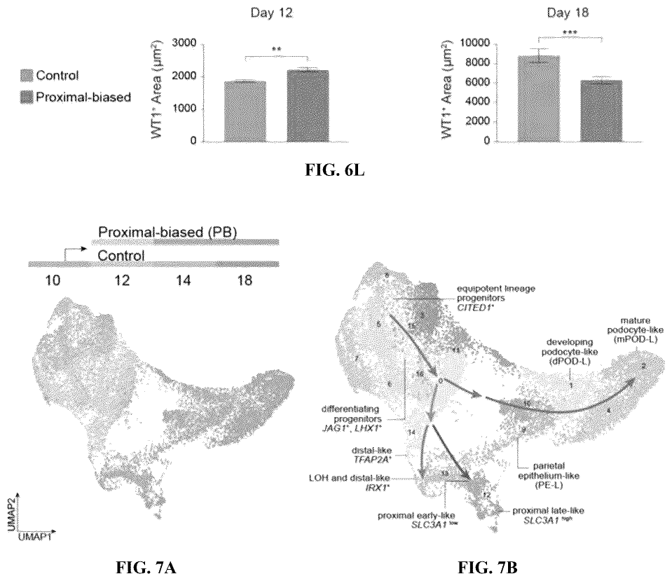

In vivo kidney datasets: In vivo datasets of human week 1418 (GSE139280) and human week 17 (GSE124472) were analyzed independently (FIGS. 2A-2L) and were used for comparison to scRNA-seq profiles of the nephrogenic subset of differentiating organoids. To identify genes that are co-expressed with HNF1B and HNF4A, a Pearson correlation test was performed for each factor, and visualized against clusters 9 (JAG1⁺, HNF1B^{LOW}, HNF4A⁻), 21 (JAG1⁺, HNF1B⁺, HNF4A^{LOW}), 24, and 20 (JAG1^{LOW}, HNF1B⁺, HNF4A⁺), which served as the early medial to proximalizing lineage (FIG. 2A, FIG. 3A). The expression of these genes in vivo and in corresponding clusters of reanalyzed kidney organoid nephrons (FIG. 2D; assembled as described below) was analyzed in FIG. 3A. Lastly, this human scRNA-seq dataset was integrated with the proximal-biased organoid dataset (FIGS. 6G-6H) using RunCCA.

In vitro organoid datasets: To unbiasedly assess transcriptional profiles of proximalizing nephron cells, the study assembled and reanalyzed differentiating kidney organoids generated from multiple sources. Data from day 25 (GSE102596); days 7, 15, and 29 (GSE136314); days 16 and 28 (GSE124472); and day 26 (GSE118184) organoids were reanalyzed. Raw data from each dataset were acquired and analyzed individually as above before being integrated. This integrated dataset was subset into nephrogenic cell types for direct comparison to in vivo nephrogenic cells (FIGS. 2A-2L). Here, clusters 7, 6, 14, 12, 8, 18, and 3 served as the proximalizing lineage (FIGS. 3A-3F). Separately, the study re-analyzed published scRNA-seq data from organoid nephrons from a protocol generating proximal tubule-enhanced organoids (GSE184928). These data were acquired and re-analyzed independently. While proximal-biased organoid nephron cells from this study were sampled for control and PB conditions at day 10 (untreated), 12, 14, and 18, representing 3, 5, 7, and 11 days after initiation of 3D organoid culture, single cells from proximal tubule-enhanced organoids were sampled at one timepoint, day 27, representing 13 days of extended monolayer culture followed by 14 days of 3D culture. The in vivo cells for this comparison were sampled from a continuum of differentiation (FIG. 6E).

Integration and batch effect correction with MNN: To analyze the expression patterns of genes associated with proximal tubule program in two organoid datasets where cells were captured using different technologies, the study employed a developing human nephron dataset (FIGS. 2A-2C) as a reference and mapped each organoid dataset to the human reference. To mitigate batch effects, the Mutual Nearest Neighbor (MNN) method was used, selected for its parsimonious correction capability and its proven efficacy in a recent benchmarking study. The study retained the shared cell types among the three datasets, specifically early tubules, podocytes, proximal tubules, and distal tubules. These shared cell types facilitated the individual alignment of each organoid dataset with the human developing kidney reference. For the alignment, the study focused on the top 3000 highly variable genes with default parameters. To statistically compare the organoid datasets with the human reference data, the study conducted a Wilcoxon Rank-sum test, incorporating a Bonferroni correction for multiple testing. The criteria for identifying significantly differentially expressed genes were set at an adjusted p-value of less than 0.05, coupled with a log fold change greater than 0.5 or less than −0.5.

Pseudotime reconstruction of lineages: Podocytes were subset from the organoid nephron single-cell RNA-sequencing dataset for a more streamlined developmental trajectory analysis from a day 10 equipotent lineage progenitor cell to a day 18 proximal-biased nephron cell. Monocle 3 was used to reconstruct the differentiation trajectory.

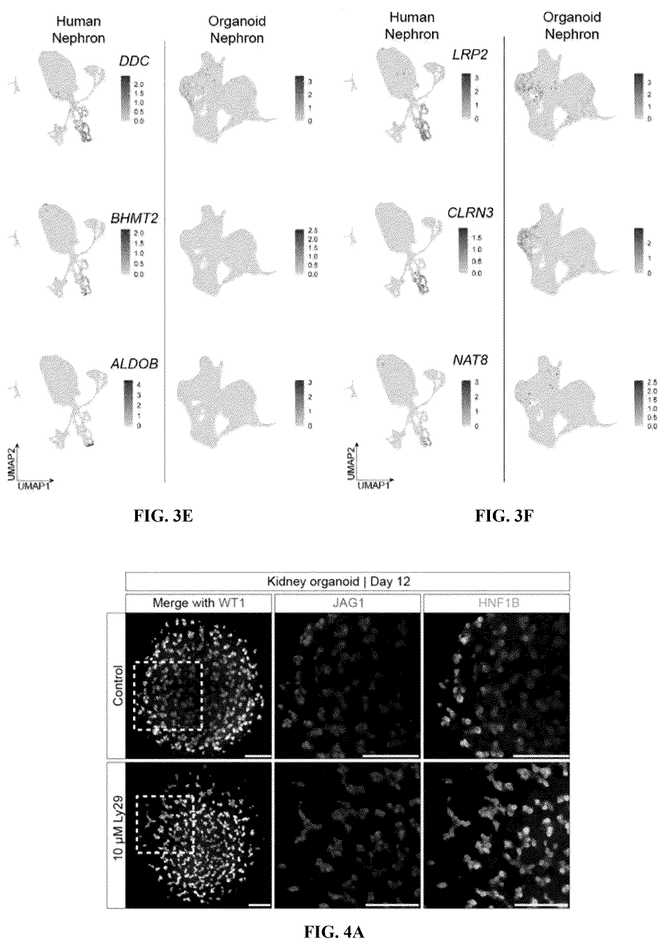

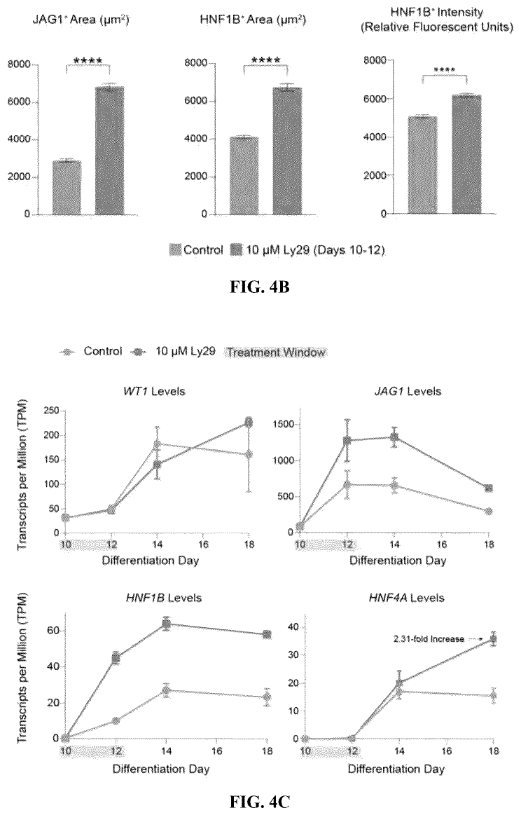

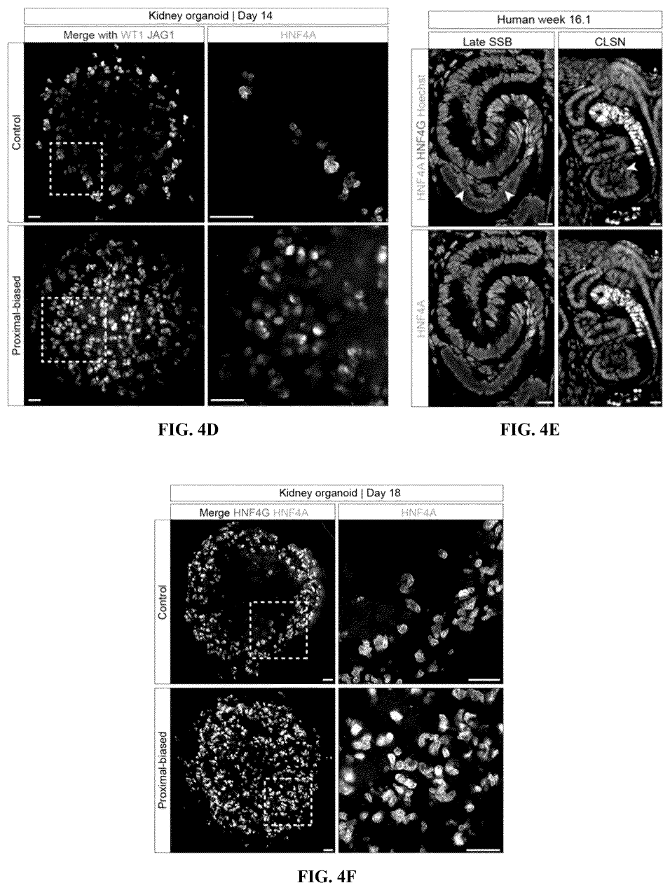

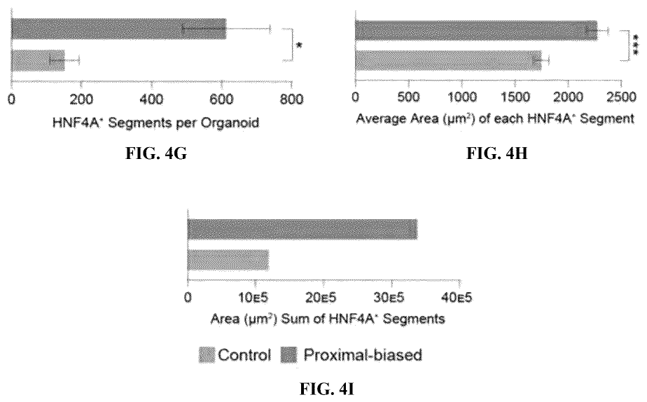

Other datasets referenced: To analyze Hnf4a-dependent gene expression in kidney organoids, the study acquired bulk RNA-sequencing data from control and Hnf4a-mutant P0 mouse kidneys (GSE144772). Bulk RNA-sequencing fastq files from two control and two mutant (Osr2Cre-driven deletion of Hnf4a) P0 kidneys were acquired. Sample quality was assessed using fastqc. Using STAR version 2.7.10a, reads were aligned to the mouse mm10 genome, Ensembl release 102. Mapping quality was appraised using qualimap. Resulting .bam files were then used to generate a feature counts table using featurecounts. A list of 257 genes that are significantly downregulated in the Hnf4a-mutant kidney (DESeq2 comparing control vs. mutant kidneys, p_{adj}<0.01, log₂FC≥1.5) were used for analysis (FIGS. 3A-3F). Separately, a list of 415 genes that are highly expressed and enriched in the adult mouse proximal tubule (GSE129798; Kidney Cell Explorer) were used for analyses in FIGS. 3A-3F. To generate the heatmaps presented in FIGS. 4B-4C, three proximalizing human clusters (FIG. 2A, clusters 20, 21, and 24) were analyzed, alongside the organoid data, which exclusively focused on cluster 3. These clusters were selected based on their relatively elevated expression levels of HNF4A, each surpassing 15% of all cells in the cluster. Human orthologs of mouse genes were retrieved using Ensembl BioMart, and genes expressed in less than three cells of the human dataset were removed.

Immunofluorescent imaging and analyses: Kidney organoids and explants. Whole kidney organoids and mouse kidney explants were fixed for 20 min on ice in 4% PFA (Electron Microscopy Sciences, 15710) in 1×PBS. In 1×PBS, organoids and explants were then carefully cut out of the permeable membrane well and placed into 1×PBS with 1.5% SEA Block (Thermo Fisher Scientific, 37527X3) and 0.1% TritonX100 (EMD Millipore, 1.08643) (blocking solution) at 4° C. with gentle movement for one hour. Primary antibodies were resuspended in blocking solution, and organoids were incubated in primary antibody and blocking solution overnight. Samples were rinsed and then washed for at least 3 hours through several rounds of 1×PBS with 0.1% TritonX100. Secondary antibodies were resuspended in blocking solution, and organoids were incubated in secondary antibody and blocking solution overnight. Rinsing and washing steps were repeated the next day before a 25 minute counterstain incubation in 1×PBS supplemented with 0.1% TritonX100 and 1 μg/mL Hoechst 33342 (Thermo Fisher Scientific, H3570) to stain nuclei. Organoids were then washed for 1 hour in 1×PBS alone. Individual organoids were mounted for imaging on slides with a glass coverslip in Immu-Mount (Thermo Fisher Scientific, 9990402). Slides were stored at 4° C. in the dark. Primary antibodies used in this study were: WT1 (abeam, ab89901, 1:1000), JAG1 (R&D Systems, AF599, 1:300), HNF1B (Thermo Fisher Scientific, MA5-24605, 1:500), HNF4A (R&D Systems, MAB4605, 1:200), CDH1 (BD Biosciences, 610181, 1:300), ZO-1 (Thermo Fisher Scientific, 33-9100, 1:200), PAX2 (R&D Systems, AF3364, 1:50), SIX1 (Cell Signaling Technology, 12891S, 1:300), HES1 (Cell Signaling Technology, 11988, 1:300), POU3F3 (Novus Biologicals, NBP1-49872, 1:500), HNF4G (Thermo Fisher Scientific, PA5-82189, 1:200), LRP2 (My Bio Source, MBS690201, 1:500), HAVCR1 (R&D Systems, AF1750, 1:200), and 7H2AX (Cell Signaling Technology, 2577). Secondary antibodies conjugated with AlexaFluor 488, 555, 594, and 647 were all diluted to 1:500 in blocking solution.

Human kidney sections: Human kidneys were carefully dissected out from donated tissues and placed in 1×PBS. Kidneys were then placed into 4% PFA to fix overnight (18 hrs). Kidneys were washed twice in 1×PBS (1 hour per wash), and samples were placed in 30% sucrose in 1×PBS rocking at 4° C. overnight (18 hrs). The next day, kidneys were swirled in Optimal Cutting Temperature (OCT from Sakura Finetech USA Inc, 4583) compound 3 times before embedding in an OCT block. The OCT blocks with tissues were frozen on a dry ice/ethanol slurry. Blocks were stored at −80° C., and ˜12 μm sections were obtained using a cryostat. Slides were stored at −80° C. before being processed for immunofluorescent imaging.

mRNA-sequencing and data analyses: Whole organoid samples (3 independent biological replicates per timepoint, per condition for differentiation days 10 and 12, and 2 replicates for differentiation days 14 and 18) were prepared and purified according to the RNeasy Mini Kit (Qiagen, 74104). Purified RNA was sent to Novogene for sequencing. Reads were aligned to the hg38 Ensembl 105 annotation using STAR version 2.7.10a. A noise-reduction filter was applied to keep genes in which the maximum count in at least one sample was greater than or equal to 10. Partek Flow version 10.0 was then used to normalize these raw feature counts following the transcripts per million (TPM) normalization method. After filtering genes with TPM values greater than or equal to 25 in at least one sample, Z-score averages were calculated and heatmaps were clustered using pheatmap. Separately, gene set enrichment analyses were performed specifically on Day 12 replicate samples by first normalizing noise-reduced feature counts using the DESeq2 median ratio method, as recommended for gene set enrichment analyses. Partek Flow version 10.0 was then subsequently used for gene set enrichment analyses. Data are deposited with GEO accession number GSE264217 and will be made publicly available as of the date of publication.

### Validation of Organoid Physiology and Injury Modeling

In vitro TRITC-albumin and dextran uptake assays: For the albumin uptake assay, differentiation day 18 control and proximal-biased organoids were incubated at 37° C./5% CO₂ for 4 hours in TeSR-E6 supplemented with 10 μg/mL TRITC-albumin (Sigma Aldrich, A2289). Control organoids were incubated with TeSR-E6 alone. After the 4 hour incubation, organoids were washed at least 3 times with TeSR-E6 media and live-imaged immediately after. Organoids were left in culture with only TeSR-E6 media overnight, and then live-imaged again the next day before being fixed and processed for immunofluorescent imaging and analyses. For the dextran uptake assay, organoids were treated and processed in the same way, except that TeSR-E6 was supplemented with Alexa-647-conjugated Dextran (Thermo Fisher Scientific, D22914).

In vitro cisplatin injury assay: For the kidney organoid cisplatin injury assay, the study adapted an approach undertaken by two recent studies. Differentiation day 18 control and proximal-biased organoids were incubated overnight (18 hrs) at 37° C./5% CO₂ in TeSR-E6 supplemented with 5 μM cisplatin (Millipore Sigma, 232120). Organoids recovered between days 19 and 20, before another overnight culture (18 hrs) between days 20 and 21 with 5 μM cisplatin. Uninjured organoids were cultured with DMSO vehicle. Organoids were then processed for immunofluorescent imaging and analyses.

Image acquisition and analysis: Image acquisition of whole-mounted organoids, cryo-sectioned human kidneys, and whole-mounted mouse kidney explants were performed using (1) Leica SP8-X confocal fluorescence imaging system (Leica Microsystems, Germany), in 1024×1024 pixels using 25× water, 40× oil, and 63× oil objectives, and (2) Leica Stellaris confocal microscope at 20× oil and 93× glycerol objectives. A Leica Thunder microscope was additionally used at 10× dry objective to image whole-mounted kidney organoids. Live organoids were captured using Zeiss Axio Zoom at 16× and 32× objectives. Image masking was performed and quantified with Imaris 9.7 software (Oxford Instruments), and with Fiji. Fiji was used for individual cell and nephron segment counting, and for quantifying relative protein signal and organoid nephron size.

### Results

Identifying abnormal kidney organoid developmental programs: To identify differences between developing nephrons in kidneys and organoids that can explain why organoids do not generate maturing proximal cells, the study characterized how proximal precursors form in vivo in developing human kidneys and compared organoids to this blueprint using single-cell RNA-sequencing and secondary validation for proteins that mark and drive proximal tubule development.

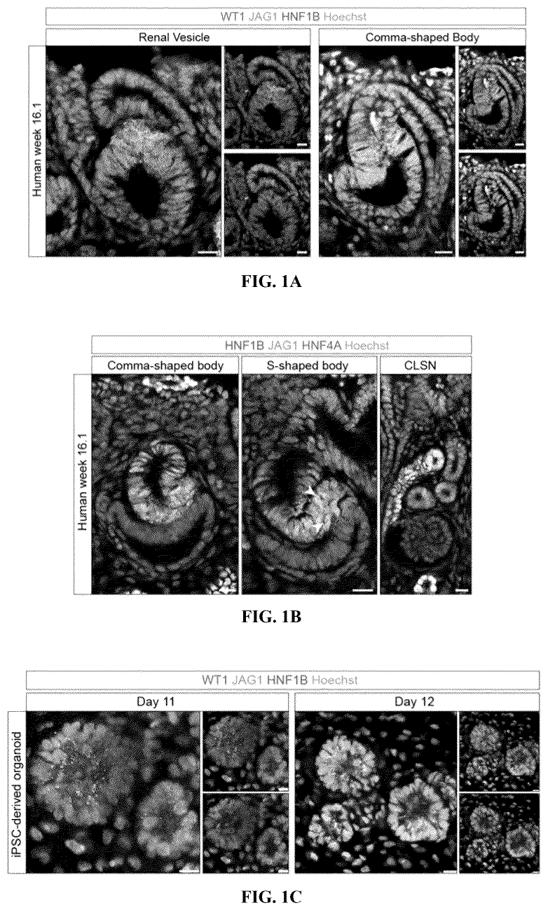

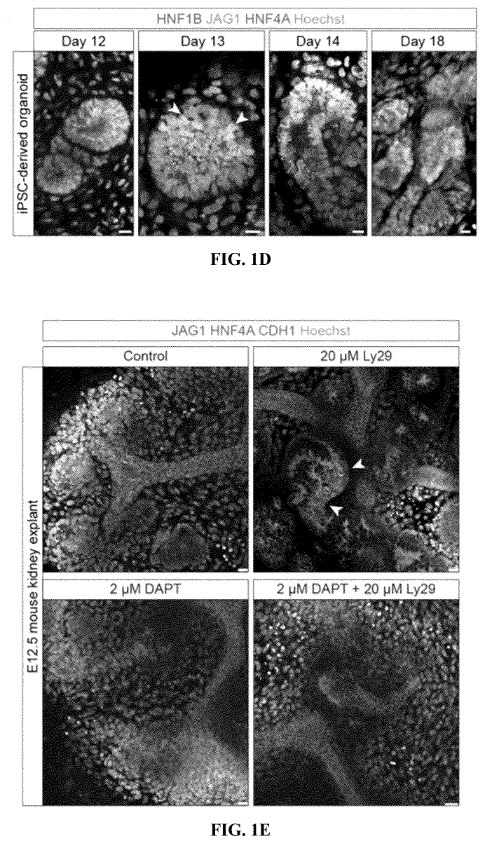

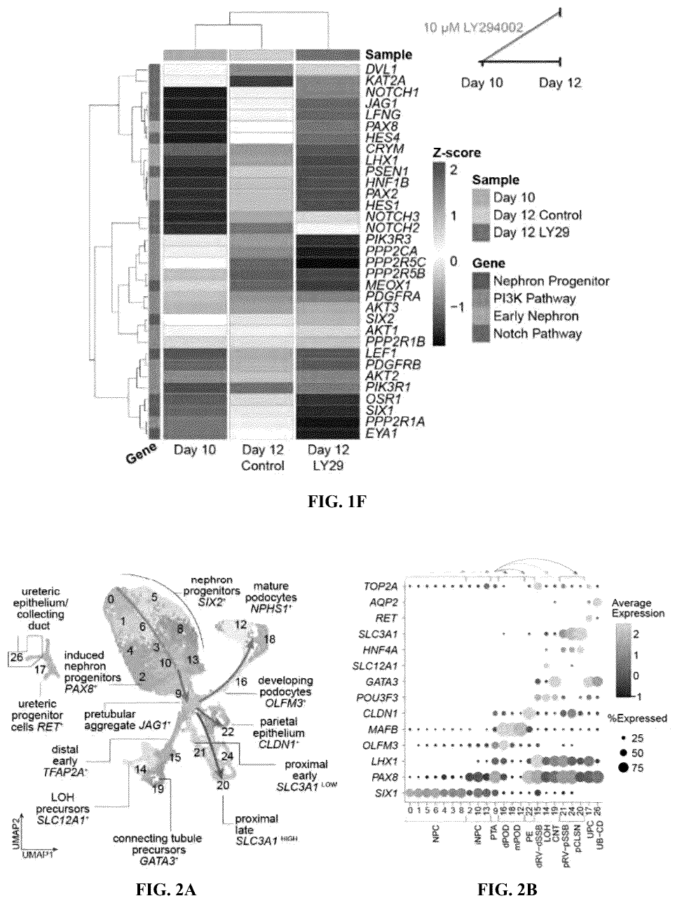

The specification of proximal precursors is consequent to the gradual recruitment of nephron progenitor cells into the forming nephron and signaling pathways that tune differentiation along the progressively emerging proximal-distal axis. This process begins with a cellular domain developing in the distal renal vesicle nephron formed by early recruited nephron progenitor cells. The domain is marked by membrane localized JAG1 and nuclear HNF1B and is in direct contact with the ureteric epithelium. It abuts the proximally located WT1⁺ region where cells are actively recruited from the nephron progenitor cell niche (FIG. 1A). As the renal vesicle develops into a comma-shaped body nephron, the HNF1B⁺/JAG1⁺ domain expands proximally. The initial distal domain downregulates JAG1 to form HNF1B⁺/JAG1^{low} cells, while the proximally expanding domain further upregulates JAG1 to become HNF1B⁺/JAG1^{high}, now considered the medial domain of the comma-shaped body. The medial domain also forms a boundary with the WT1⁺ proximal-most domain where the last nephron progenitors are recruited (FIG. 1A, FIGS. 2A-2C). The nephron is at the comma-shaped nephron stage, subdivided by these markers into three distinguishable domains. As it develops further, HNF1B is additionally upregulated in nuclei positioned at the border between the medial and proximal-most domain, where HNF4A is detected in late comma-shaped/early S-shaped body nephrons (FIG. 1B). Proximal tubule precursor cells (HNF1B^{high}/HNF4A⁺) then develop and downregulate JAG1 as the S-shaped nephron matures into capillary loop stage nephrons, when an elongating tubule forms with a HNF1B^{high}/HNF4A^{high}/JAG1⁻ proximal cell state (FIG. 1B). These finds are consistent with single-cell RNA-sequencing of the developing human nephron lineage (FIGS. 2A-2C). Proximal precursor cells diverge from other nephron lineages in an early PAX8⁺ cell population in the pretubular aggregate, which generates WT1⁺ podocyte precursors, TFAP2A⁺ distal precursors, and JAG1⁺ cells that sequentially upregulate HNF1B and HNF4A (FIGS. 2A-2C).

Organoid nephrons form differently: At day 10, kidney organoids include individual cell-aggregates positive for nephron progenitor markers (WT1, SIX1) and nephron lineage marker (PAX2), that coalesce around a forming apical epithelial polarity (CDH1, ZO1). As the aggregates further epithelialize (day 11-12), they display uniform deposition of WT1 in nuclei, and JAG1 as puncta and weakly at cell-membranes around the periphery of the forming nephron (FIGS. 1C-1D, FIGS. 2G-2H). HNF1B is upregulated between days 11 and 12 and is deposited in nuclei around the periphery of each nephron. At these stages, there are no indications of WT1, JAG1, and HNF1B being distributed along a gradually forming proximal-distal axis as seen in vivo (FIG. 1A), rather the organoid nephrons exhibit a HNF1B⁺/JAG1⁺/WT1⁺ triple-positive cell state (FIG. 1C). During further differentiation, organoid nephrons generate HNF1B⁺/HNF4A⁺ cells, but these do not transition into a rapidly elongating phase as observed in vivo and JAG1 remains strongly detectable (FIG. 1B, FIG. 1D). Single-cell data from organoids sampled over time and differentiation protocols indicate that the abnormal early and late triple-positive PAX8⁺/JAG1⁺/WT1⁺ and late HNF1B⁺/HNF4A⁺/JAG1⁺ cell states are a common trend across models (reanalyzed and combined from several sources) (FIGS. 2D-2F).

Given the importance of Hnf1b and Hnf4a for normal proximal tubule development, the study examined the possibility that organoids abnormally regulate functionally important Hnf1b/HNF1B and Hnf4a/HNF4A-mediated transcriptional programs and assessed organoid proximal fate development against the in vivo developmental blueprint. To identify genes that are co-expressed with each transcription factor in vivo (FIG. 2A), a Pearson expression correlation analysis was performed for each gene, and the expression of HNF1B and HNF4A correlates was characterized. The expression of these genes in vivo was ordered along a predicted proximal developmental trajectory, and their expression patterns highlight a transition from HNF1B correlates in early cells (cluster 9: pretubular aggregate/renal vesicle; e.g., JAG1, HES1, KRT8) through to a gradually more HNF4A-correlating signature (clusters 21, 24: S-shaped body; e.g., CLU, DCDC2, ANXA4), and finally genes strongly enriched in the maturing proximal precursors (cluster 20: Capillary loop stage nephron; e.g., ASS1, SLC34A1, SLC22A8; FIG. 3A (left)).

Using this progression as a framework for the temporal in vivo sequence of transcriptional events, the study compared it to organoid proximal precursor development across models (FIG. 3A (right) based on FIG. 2D). In vitro, HNF1B-correlates such as JAG1, HES1, KIF12, and KRT8 were detected while other genes, for instance transcription factor ELF3, fibroblast growth factor receptor FGFR4, serine protease inhibitor SERPINF2, and extracellular signaling protein CYR61 were not. This partial recapitulation of the transcription profile was further reduced for HNF4A-correlates, with only 25/46 genes detected across in vitro models. Genes coding for a range of protein types were not detected or significantly expressed, for instance solute carriers SLC22A8, SLC5A8, SLC16A9, and transferases and enzymes such as AGXT2, GLYAT, and ANPEP.

To independently examine whether HNF4A-dependent genes are underrepresented in the organoid proximal program, genes downregulated upon in vivo Hnf4a loss-of-function were intersected with transcriptional profiles from detailed expression maps of the adult male and female mouse kidney. The study categorized Hnf4a-dependent genes as those expressed only in development and those that persist into the adult functional nephron. 257 genes are significantly downregulated (control vs. mutant kidneys, padj<0.01, log₂FC≥1.5) in postnatal day 0 animals on loss of Hnf4a, and 415 genes are enriched in the adult male and female proximal tubules (Kidney Cell Explorer) (FIG. 3B). Of the 257 Hnf4a-dependent genes, 124 genes were enriched only in the developing kidney while 133 genes showed persistent expression into functional adult nephrons. To determine whether human orthologs are expressed in the developing human proximal tubule and in organoid models, the gene lists were intersected with single-cell transcriptional data (FIG. 2A, FIG. 2D). Of the 133 Hnf4a-dependent genes expressed during development and in adult mouse nephrons, 92 human orthologs were detected. 91/92 were also detected in organoids, and 89/92 (96.7%) were detected at greater abundance in vivo (FIG. 3C, FIG. 3E, TABLE 1, TABLE 2). Similarly, of the 124 Hnf4a-dependent genes expressed during development, 81 human orthologs were detected. 80 of these were detected in organoids, but 70/81 (86.4%) were again detected at a higher frequency in vivo (FIG. 3D, FIG. 3F, TABLE 3, TABLE 4).

TABLE 1
Human orthologs of the 133 mouse genes depicted in the Venn
diagram in FIG. 3B. Counts represent the total number of cells
within proximal clusters (defined as ≥15% of cells expressing
HNF4A) expressing each gene in either the human nephron (FIGS.
2A-2C) or organoid nephron merged (FIGS. 2D-2F) objects. The
percentage of all cells expressing the specified gene within
these proximal clusters is also provided. These percentages
were utilized to generate the heatmap in FIG. 3C.

| | | Organoid | |
|---|---|---|---|
| Human Ortholog | Human Count | Cluster 3 Count | Human Total Cells |
| SLC3A1 | 640 | 2201 | 814 |
| CRYL1 | 617 | 2393 | 814 |
| CNDP2 | 580 | 1486 | 814 |
| ASS1 | 572 | 1855 | 814 |
| SLC27A2 | 553 | 1611 | 814 |
| GPX3 | 546 | 2178 | 814 |
| ACMSD | 530 | 905 | 814 |
| HGD | 512 | 1763 | 814 |
| ECI2 | 503 | 1876 | 814 |
| FMO1 | 472 | 2324 | 814 |
| DDC | 465 | 747 | 814 |
| GATM | 428 | 1930 | 814 |
| SERPINF2 | 428 | 864 | 814 |
| SLC16A4 | 402 | 1537 | 814 |
| SLC13A1 | 396 | 987 | 814 |
| APOM | 393 | 1671 | 814 |
| AK4 | 385 | 1360 | 814 |
| GLYAT | 365 | 659 | 814 |
| ACY3 | 364 | 326 | 814 |
| SORD | 297 | 280 | 814 |
| SLC51B | 278 | 1053 | 814 |
| SLC34A1 | 273 | 664 | 814 |
| FBP1 | 269 | 376 | 814 |
| AGXT2 | 259 | 664 | 814 |
| HAO2 | 257 | 306 | 814 |
| SLC6A13 | 257 | 830 | 814 |
| METTL7A | 252 | 547 | 814 |
| PRODH2 | 247 | 418 | 814 |
| GGT1 | 240 | 679 | 814 |
| PCK1 | 237 | 243 | 814 |
| SLC5A8 | 235 | 595 | 814 |
| SUSD3 | 222 | 779 | 814 |
| SLC7A7 | 217 | 1005 | 814 |
| BHMT2 | 212 | 7 | 814 |
| NAT8 | 210 | 121 | 814 |
| ALPL | 198 | 357 | 814 |
| SLC5A2 | 172 | 24 | 814 |
| SLC5A12 | 153 | 138 | 814 |
| SLC22A8 | 145 | 163 | 814 |
| SLC17A1 | 139 | 405 | 814 |
| SLC7A9 | 130 | 201 | 814 |
| EHHADH | 123 | 148 | 814 |
| CDHR2 | 122 | 240 | 814 |
| TMEM106A | 111 | 209 | 814 |
| ALDOB | 102 | 48 | 814 |
| CLEC2D | 99 | 308 | 814 |
| CHRNA4 | 95 | 315 | 814 |
| SLC23A1 | 93 | 163 | 814 |
| ACOX2 | 90 | 150 | 814 |
| SLC34A3 | 85 | 148 | 814 |
| SLC2A2 | 83 | 25 | 814 |
| NAPSA | 82 | 153 | 814 |
| SLC22A6 | 82 | 66 | 814 |
| PAH | 79 | 25 | 814 |
| ASPDH | 76 | 154 | 814 |
| PYROXD2 | 76 | 160 | 814 |
| AADAT | 68 | 223 | 814 |
| DMGDH | 66 | 66 | 814 |
| SMLR1 | 66 | 83 | 814 |
| GPM6A | 56 | 106 | 814 |
| ACOT4 | 55 | 145 | 814 |
| GJB2 | 54 | 53 | 814 |
| ACE | 53 | 256 | 814 |
| SUSD2 | 46 | 45 | 814 |
| IYD | 45 | 190 | 814 |
| ERICH4 | 42 | 120 | 814 |
| SLC22A2 | 38 | 65 | 814 |
| CGREF1 | 31 | 67 | 814 |
| CYP4A11 | 31 | 2 | 814 |
| SLC23A3 | 29 | 26 | 814 |
| SLC26A1 | 29 | 69 | 814 |
| SYPL2 | 26 | 38 | 814 |
| SLC13A3 | 23 | 110 | 814 |
| SLC22A12 | 23 | 25 | 814 |
| HYKK | 22 | 67 | 814 |
| SLC51A | 22 | 75 | 814 |
| MIOX | 21 | 62 | 814 |
| ACSM1 | 17 | 59 | 814 |
| SLC6A18 | 17 | 7 | 814 |
| SLC5A11 | 16 | 119 | 814 |
| AADAC | 15 | 7 | 814 |
| SUCNR1 | 13 | 145 | 814 |
| SLC10A2 | 12 | 51 | 814 |
| GAS2 | 9 | 197 | 814 |
| LPAR3 | 7 | 4 | 814 |
| PAQR9 | 7 | 29 | 814 |
| SLC17A3 | 6 | 22 | 814 |
| SLC7A13 | 5 | 0 | 814 |
| TMIGD1 | 4 | 5 | 814 |
| TTC36 | 4 | 14 | 814 |
| CYP2J2 | 3 | 4 | 814 |
| SLC22A1 | 3 | 17 | 814 |

TABLE 2
Human orthologs of the 133 mouse genes depicted in the Venn
diagram in FIG. 3B. Counts represent the total number of cells
within proximal clusters (defined as ≥15% of cells expressing
HNF4A) expressing each gene in either the human nephron (FIGS.
2A-2C) or organoid nephron merged (FIGS. 2D-2F) objects. The
percentage of all cells expressing the specified gene within
these proximal clusters is also provided. These percentages
were utilized to generate the heatmap in FIG. 3C.

| Human | Organoid Cluster | Human | Organoid |
|---|---|---|---|
| Ortholog | 3 Total Cells | Percentage | Percentage |
| SLC3A1 | 4625 | 78.6240786 | 47.5891892 |
| CRYL1 | 4625 | 75.7985258 | 51.7405405 |
| CNDP2 | 4625 | 71.2530713 | 32.1297297 |
| ASS1 | 4625 | 70.2702703 | 40.1081081 |
| SLC27A2 | 4625 | 67.9361179 | 34.8324324 |
| GPX3 | 4625 | 67.0761671 | 47.0918919 |
| ACMSD | 4625 | 65.1105651 | 19.5675676 |
| HGD | 4625 | 62.8992629 | 38.1189189 |
| ECI2 | 4625 | 61.7936118 | 40.5621622 |
| FMO1 | 4625 | 57.985258 | 50.2486486 |
| DDC | 4625 | 57.1253071 | 16.1513514 |
| GATM | 4625 | 52.5798526 | 41.7297297 |
| SERPINF2 | 4625 | 52.5798526 | 18.6810811 |
| SLC16A4 | 4625 | 49.3857494 | 33.2324324 |
| SLC13A1 | 4625 | 48.6486486 | 21.3405405 |
| APOM | 4625 | 48.2800983 | 36.1297297 |
| AK4 | 4625 | 47.2972973 | 29.4054054 |
| GLYAT | 4625 | 44.8402948 | 14.2486486 |
| ACY3 | 4625 | 44.7174447 | 7.04864865 |
| SORD | 4625 | 36.4864865 | 6.05405405 |
| SLC51B | 4625 | 34.1523342 | 22.7675676 |
| SLC34A1 | 4625 | 33.5380835 | 14.3567568 |
| FBP1 | 4625 | 33.046683 | 8.12972973 |
| AGXT2 | 4625 | 31.8181818 | 14.3567568 |
| HAO2 | 4625 | 31.5724816 | 6.61621622 |
| SLC6A13 | 4625 | 31.5724816 | 17.9459459 |
| METTL7A | 4625 | 30.958231 | 11.827027 |
| PRODH2 | 4625 | 30.3439803 | 9.03783784 |
| GGT1 | 4625 | 29.4840295 | 14.6810811 |
| PCK1 | 4625 | 29.1154791 | 5.25405405 |
| SLC5A8 | 4625 | 28.8697789 | 12.8648649 |
| SUSD3 | 4625 | 27.2727273 | 16.8432432 |
| SLC7A7 | 4625 | 26.6584767 | 21.7297297 |
| BHMT2 | 4625 | 26.044226 | 0.15135135 |
| NAT8 | 4625 | 25.7985258 | 2.61621622 |
| ALPL | 4625 | 24.3243243 | 7.71891892 |
| SLC5A2 | 4625 | 21.1302211 | 0.51891892 |
| SLC5A12 | 4625 | 18.7960688 | 2.98378378 |
| SLC22A8 | 4625 | 17.8132678 | 3.52432432 |
| SLC17A1 | 4625 | 17.0761671 | 8.75675676 |
| SLC7A9 | 4625 | 15.970516 | 4.34594595 |
| EHHADH | 4625 | 15.1105651 | 3.2 |
| CDHR2 | 4625 | 14.987715 | 5.18918919 |
| TMEM106A | 4625 | 13.6363636 | 4.51891892 |
| ALDOB | 4625 | 12.5307125 | 1.03783784 |
| CLEC2D | 4625 | 12.1621622 | 6.65945946 |
| CHRNA4 | 4625 | 11.6707617 | 6.81081081 |
| SLC23A1 | 4625 | 11.4250614 | 3.52432432 |
| ACOX2 | 4625 | 11.0565111 | 3.24324324 |
| SLC34A3 | 4625 | 10.4422604 | 3.2 |
| SLC2A2 | 4625 | 10.1965602 | 0.54054054 |
| NAPSA | 4625 | 10.0737101 | 3.30810811 |
| SLC22A6 | 4625 | 10.0737101 | 1.42702703 |
| PAH | 4625 | 9.70515971 | 0.54054054 |
| ASPDH | 4625 | 9.33660934 | 3.32972973 |
| PYROXD2 | 4625 | 9.33660934 | 3.45945946 |
| AADAT | 4625 | 8.35380835 | 4.82162162 |
| DMGDH | 4625 | 8.10810811 | 1.42702703 |
| SMLR1 | 4625 | 8.10810811 | 1.79459459 |
| GPM6A | 4625 | 6.87960688 | 2.29189189 |
| ACOT4 | 4625 | 6.75675676 | 3.13513514 |
| GJB2 | 4625 | 6.63390663 | 1.14594595 |
| ACE | 4625 | 6.51105651 | 5.53513514 |
| SUSD2 | 4625 | 5.65110565 | 0.97297297 |
| IYD | 4625 | 5.52825553 | 4.10810811 |
| ERICH4 | 4625 | 5.15970516 | 2.59459459 |
| SLC22A2 | 4625 | 4.66830467 | 1.40540541 |
| CGREF1 | 4625 | 3.80835381 | 1.44864865 |
| CYP4A11 | 4625 | 3.80835381 | 0.04324324 |
| SLC23A3 | 4625 | 3.56265356 | 0.56216216 |
| SLC26A1 | 4625 | 3.56265356 | 1.49189189 |
| SYPL2 | 4625 | 3.19410319 | 0.82162162 |
| SLC13A3 | 4625 | 2.82555283 | 2.37837838 |
| SLC22A12 | 4625 | 2.82555283 | 0.54054054 |
| HYKK | 4625 | 2.7027027 | 1.44864865 |
| SLC51A | 4625 | 2.7027027 | 1.62162162 |
| MIOX | 4625 | 2.57985258 | 1.34054054 |
| ACSM1 | 4625 | 2.08845209 | 1.27567568 |
| SLC6A18 | 4625 | 2.08845209 | 0.15135135 |
| SLC5A11 | 4625 | 1.96560197 | 2.57297297 |
| AADAC | 4625 | 1.84275184 | 0.15135135 |
| SUCNR1 | 4625 | 1.5970516 | 3.13513514 |
| SLC10A2 | 4625 | 1.47420147 | 1.1027027 |
| GAS2 | 4625 | 1.10565111 | 4.25945946 |
| LPAR3 | 4625 | 0.85995086 | 0.08648649 |
| PAQR9 | 4625 | 0.85995086 | 0.62702703 |
| SLC17A3 | 4625 | 0.73710074 | 0.47567568 |
| SLC7A13 | 4625 | 0.61425061 | 0 |
| TMIGD1 | 4625 | 0.49140049 | 0.10810811 |
| TTC36 | 4625 | 0.49140049 | 0.3027027 |
| CYP2J2 | 4625 | 0.36855037 | 0.08648649 |
| SLC22A1 | 4625 | 0.36855037 | 0.36756757 |

TABLE 3
Human orthologs of the 124 mouse genes depicted in the Venn
diagram in FIG. 3B. Counts represent the total number of cells
within proximal clusters (defined as ≥15% of cells expressing
HNF4A) expressing each gene in either the human nephron (FIGS.
2A-2C) or organoid nephron merged (FIGS. 2D-2F) objects. The
percentage of all cells expressing the specified gene within
these proximal clusters is also provided. These percentages
were utilized to generate the heatmap in FIG. 3D.

| | | Organoid | |
|---|---|---|---|
| Human Ortholog | Human Count | Cluster 3 Count | Human Total Cells |
| QPRT | 766 | 3503 | 814 |
| C11orf54 | 723 | 2558 | 814 |
| LRP2 | 567 | 1861 | 814 |
| USH1C | 542 | 1550 | 814 |
| CUBN | 523 | 2470 | 814 |
| SMIM24 | 518 | 3790 | 814 |
| PDZK1 | 508 | 2167 | 814 |
| TMEM150A | 443 | 1601 | 814 |
| GALNT11 | 418 | 1548 | 814 |
| KCNJ15 | 412 | 2246 | 814 |
| CMBL | 387 | 715 | 814 |
| CLRN3 | 386 | 990 | 814 |
| VIL1 | 366 | 1646 | 814 |
| SLC39A5 | 365 | 1597 | 814 |
| TCEA3 | 355 | 1093 | 814 |
| TFEC | 337 | 1069 | 814 |
| FAH | 329 | 705 | 814 |
| A1CF | 311 | 844 | 814 |
| ACAT2 | 309 | 1109 | 814 |
| GSS | 303 | 760 | 814 |
| CIDEB | 296 | 1010 | 814 |
| GLYCTK | 294 | 418 | 814 |
| TST | 285 | 689 | 814 |
| GIPC2 | 276 | 1201 | 814 |
| F10 | 256 | 1263 | 814 |
| ASB9 | 254 | 500 | 814 |
| DNAJC22 | 254 | 457 | 814 |
| CFI | 252 | 828 | 814 |
| METTL7B | 242 | 622 | 814 |
| GPD1 | 240 | 786 | 814 |
| AMN | 237 | 1881 | 814 |
| CDHR5 | 236 | 768 | 814 |
| ACOT2 | 223 | 305 | 814 |
| NR1H4 | 223 | 418 | 814 |
| NAT8 | 210 | 121 | 814 |
| PDZD3 | 199 | 282 | 814 |
| AGMAT | 197 | 573 | 814 |
| ASPA | 175 | 432 | 814 |
| UGT3A2 | 158 | 298 | 814 |
| HMOX1 | 141 | 273 | 814 |
| PTER | 141 | 410 | 814 |
| THNSL2 | 138 | 477 | 814 |
| CLDN2 | 136 | 854 | 814 |
| TMEM174 | 128 | 146 | 814 |
| TINAG | 122 | 1604 | 814 |
| ANKS4B | 115 | 502 | 814 |
| LRRC19 | 115 | 738 | 814 |
| TKFC | 113 | 330 | 814 |
| SLC22A18 | 97 | 762 | 814 |
| SUGCT | 93 | 248 | 814 |
| MFSD4B | 90 | 129 | 814 |
| CALML4 | 81 | 303 | 814 |
| XPNPEP2 | 79 | 154 | 814 |
| MYO7B | 76 | 669 | 814 |
| TMEM82 | 74 | 252 | 814 |
| ABCC2 | 68 | 247 | 814 |
| SLC1A1 | 63 | 325 | 814 |
| PC | 59 | 261 | 814 |
| SLC5A9 | 57 | 81 | 814 |
| XYLB | 56 | 148 | 814 |
| UNC5CL | 53 | 225 | 814 |
| ACSM5 | 50 | 82 | 814 |
| FADS3 | 48 | 94 | 814 |
| ZMYND10 | 47 | 62 | 814 |
| HSD17B2 | 46 | 103 | 814 |
| AFM | 42 | 0 | 814 |
| MYO15B | 32 | 108 | 814 |
| DNAJC6 | 30 | 50 | 814 |
| PKLR | 30 | 105 | 814 |
| ACOT1 | 27 | 49 | 814 |
| PIPOX | 20 | 54 | 814 |
| SOD3 | 13 | 61 | 814 |
| ANGPTL4 | 11 | 144 | 814 |
| DAO | 11 | 2 | 814 |
| SLC2A5 | 11 | 46 | 814 |
| CFB | 9 | 9 | 814 |
| GALNT6 | 8 | 10 | 814 |
| MISP3 | 8 | 17 | 814 |
| SERPINA6 | 7 | 43 | 814 |
| GCNT1 | 5 | 33 | 814 |
| FMO2 | 3 | 51 | 814 |

TABLE 4
Human orthologs of the 124 mouse genes depicted in the Venn
diagram in FIG. 3B. Counts represent the total number of cells
within proximal clusters (defined as ≥15% of cells expressing
HNF4A) expressing each gene in either the human nephron (FIGS.
2A-2C) or organoid nephron merged (FIGS. 2D-2F) objects. The
percentage of all cells expressing the specified gene within
these proximal clusters is also provided. These percentages
were utilized to generate the heatmap in FIG. 3D.

| Human | Organoid Cluster | Human | Organoid |
|---|---|---|---|
| Ortholog | 3 Total Cells | Percentage | Percentage |
| QPRT | 4625 | 94.1031941 | 75.7405405 |
| C11orf54 | 4625 | 88.8206388 | 55.3081081 |
| LRP2 | 4625 | 69.6560197 | 40.2378378 |
| USH1C | 4625 | 66.5847666 | 33.5135135 |
| CUBN | 4625 | 64.2506143 | 53.4054054 |
| SMIM24 | 4625 | 63.6363636 | 81.9459459 |
| PDZK1 | 4625 | 62.4078624 | 46.8540541 |
| TMEM150A | 4625 | 54.4226044 | 34.6162162 |
| GALNT11 | 4625 | 51.3513514 | 33.4702703 |
| KCNJ15 | 4625 | 50.6142506 | 48.5621622 |
| CMBL | 4625 | 47.5429975 | 15.4594595 |
| CLRN3 | 4625 | 47.4201474 | 21.4054054 |
| VIL1 | 4625 | 44.963145 | 35.5891892 |
| SLC39A5 | 4625 | 44.8402948 | 34.5297297 |
| TCEA3 | 4625 | 43.6117936 | 23.6324324 |
| TFEC | 4625 | 41.4004914 | 23.1135135 |
| FAH | 4625 | 40.4176904 | 15.2432432 |
| A1CF | 4625 | 38.2063882 | 18.2486486 |
| ACAT2 | 4625 | 37.960688 | 23.9783784 |
| GSS | 4625 | 37.2235872 | 16.4324324 |
| CIDEB | 4625 | 36.3636364 | 21.8378378 |
| GLYCTK | 4625 | 36.1179361 | 9.03783784 |
| TST | 4625 | 35.012285 | 14.8972973 |
| GIPC2 | 4625 | 33.9066339 | 25.9675676 |
| F10 | 4625 | 31.4496314 | 27.3081081 |
| ASB9 | 4625 | 31.2039312 | 10.8108108 |
| DNAJC22 | 4625 | 31.2039312 | 9.88108108 |
| CFI | 4625 | 30.958231 | 17.9027027 |
| METTL7B | 4625 | 29.7297297 | 13.4486486 |
| GPD1 | 4625 | 29.4840295 | 16.9945946 |
| AMN | 4625 | 29.1154791 | 40.6702703 |
| CDHR5 | 4625 | 28.992629 | 16.6054054 |
| ACOT2 | 4625 | 27.3955774 | 6.59459459 |
| NR1H4 | 4625 | 27.3955774 | 9.03783784 |
| NAT8 | 4625 | 25.7985258 | 2.61621622 |
| PDZD3 | 4625 | 24.4471744 | 6.0972973 |
| AGMAT | 4625 | 24.2014742 | 12.3891892 |
| ASPA | 4625 | 21.4987715 | 9.34054054 |
| UGT3A2 | 4625 | 19.4103194 | 6.44324324 |
| HMOX1 | 4625 | 17.3218673 | 5.9027027 |
| PTER | 4625 | 17.3218673 | 8.86486486 |
| THNSL2 | 4625 | 16.953317 | 10.3135135 |
| CLDN2 | 4625 | 16.7076167 | 18.4648649 |
| TMEM174 | 4625 | 15.7248157 | 3.15675676 |
| TINAG | 4625 | 14.987715 | 34.6810811 |
| ANKS4B | 4625 | 14.1277641 | 10.8540541 |
| LRRC19 | 4625 | 14.1277641 | 15.9567568 |
| TKFC | 4625 | 13.8820639 | 7.13513514 |
| SLC22A18 | 4625 | 11.9164619 | 16.4756757 |
| SUGCT | 4625 | 11.4250614 | 5.36216216 |
| MFSD4B | 4625 | 11.0565111 | 2.78918919 |
| CALML4 | 4625 | 9.95085995 | 6.55135135 |
| XPNPEP2 | 4625 | 9.70515971 | 3.32972973 |
| MYO7B | 4625 | 9.33660934 | 14.4648649 |
| TMEM82 | 4625 | 9.09090909 | 5.44864865 |
| ABCC2 | 4625 | 8.35380835 | 5.34054054 |
| SLC1A1 | 4625 | 7.73955774 | 7.02702703 |
| PC | 4625 | 7.24815725 | 5.64324324 |
| SLC5A9 | 4625 | 7.002457 | 1.75135135 |
| XYLB | 4625 | 6.87960688 | 3.2 |
| UNC5CL | 4625 | 6.51105651 | 4.86486486 |
| ACSM5 | 4625 | 6.14250614 | 1.77297297 |
| FADS3 | 4625 | 5.8968059 | 2.03243243 |
| ZMYND10 | 4625 | 5.77395577 | 1.34054054 |
| HSD17B2 | 4625 | 5.65110565 | 2.22702703 |
| AFM | 4625 | 5.15970516 | 0 |
| MYO15B | 4625 | 3.93120393 | 2.33513514 |
| DNAJC6 | 4625 | 3.68550369 | 1.08108108 |
| PKLR | 4625 | 3.68550369 | 2.27027027 |
| ACOT1 | 4625 | 3.31695332 | 1.05945946 |
| PIPOX | 4625 | 2.45700246 | 1.16756757 |
| SOD3 | 4625 | 1.5970516 | 1.31891892 |
| ANGPTL4 | 4625 | 1.35135135 | 3.11351351 |
| DAO | 4625 | 1.35135135 | 0.04324324 |
| SLC2A5 | 4625 | 1.35135135 | 0.99459459 |
| CFB | 4625 | 1.10565111 | 0.19459459 |
| GALNT6 | 4625 | 0.98280098 | 0.21621622 |
| MISP3 | 4625 | 0.98280098 | 0.36756757 |
| SERPINA6 | 4625 | 0.85995086 | 0.92972973 |
| GCNT1 | 4625 | 0.61425061 | 0.71351351 |
| FMO2 | 4625 | 0.36855037 | 1.1027027 |

These data show that correlates of HNF4A and human orthologs of Hnf4a-dependent genes are expressed infrequently in organoids. This is consistent across organoid models, collectively pointing to abnormal regulation of HNF4A-mediated gene expression.

Initiating a proximal-forming cell-state in kidney organoids: To develop a strategy to drive proximal precursor development in organoids, the study revisited previous work identifying a relationship between PI3K signaling and Notch ligand Jag). In mouse kidneys, WNT/β-catenin and PI3K signaling have opposing effects on Jag) expression, and pharmacological inhibition of PI3K signaling results in rapid upregulation of Jag) in nephron progenitors and early nephrons. The study therefore tested whether inhibition of PI3K and upregulation Jag) could drive a proximal nephron program as this could provide a tool to generate human proximal tubule precursors in kidney organoids. Mouse kidney explants were cultured with PI3K inhibitor LY294002 (Ly29) for 48 hours. This increased Jag) abundance. Hnf4a was co-detected in Jag1⁺ cells, confirming expression of a bona fide proximal precursor marker (FIG. 1E). To determine if Hnf4a expression was dependent on Notch signaling, as suggested by mouse genetic models, kidneys were treated with both Ly29 and Notch/gamma-secretase inhibitor DAPT. Co-inhibition of PI3K and Notch signaling blocked Hnf4a expression, as did inhibiting Notch on its own (FIG. 1E). These data confirm an interaction between the PI3K and Notch pathways in generating proximal cell identities in kidneys.

To validate this strategy in kidney organoids, organoids were treated with Ly29 from days 10-12. This is the time when JAG1 is partially upregulated in vitro and when nephrons epithelialize. They were transcriptionally profiled before and after treatment using RNA-sequencing. PI3K inhibitor-treated organoids displayed a 1.78-fold increase in JAG1 expression, and Notch pathway genes LNFG, HES1, HES4, NOTCH1 were strongly upregulated, as were differentiation markers PAX8 and LHX1. Nephron progenitor markers SIX1, MEOX1, OSR1, EYA1 were downregulated. Wnt/β-catenin targets WNT4 and LGR5 were unchanged (FIG. 1F, FIG. 2I). Gene set enrichment analyses confirmed that the Notch pathway was selectively upregulated, PI3K pathway downregulated, and other unrelated pathways, e.g., Hedgehog, were unaffected (FIGS. 2J-2L). Within this 48-hr. timeframe, HNF1B expression was upregulated 2.88-fold in PI3K inhibited samples, but HNF4A was not detected at day 12 (TPM<1.7; FIG. 1F, FIG. 2I). These data show that organoids upregulate JAG1 and HNF1B in response to transient PI3K inhibition.

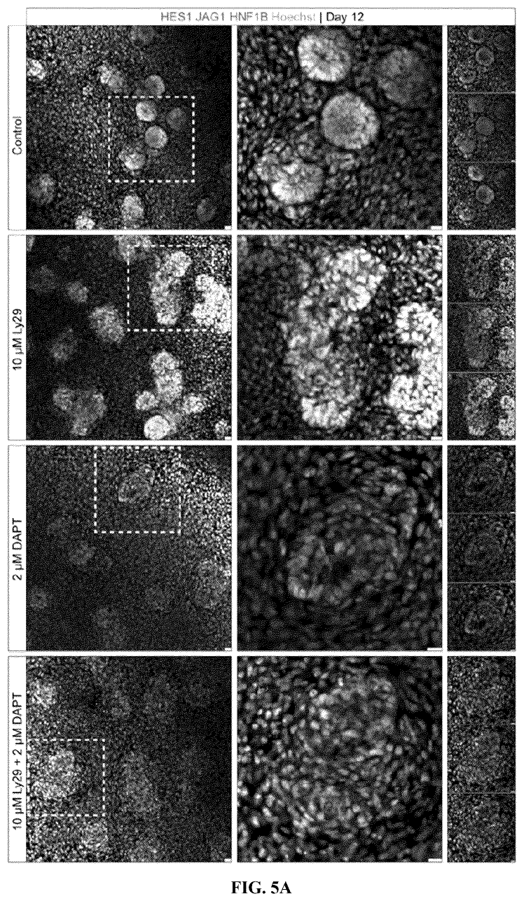

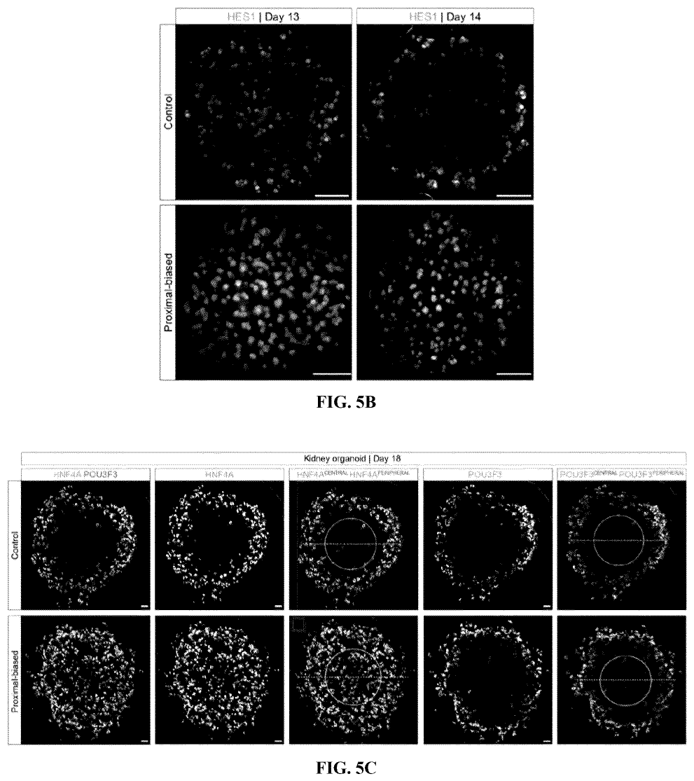

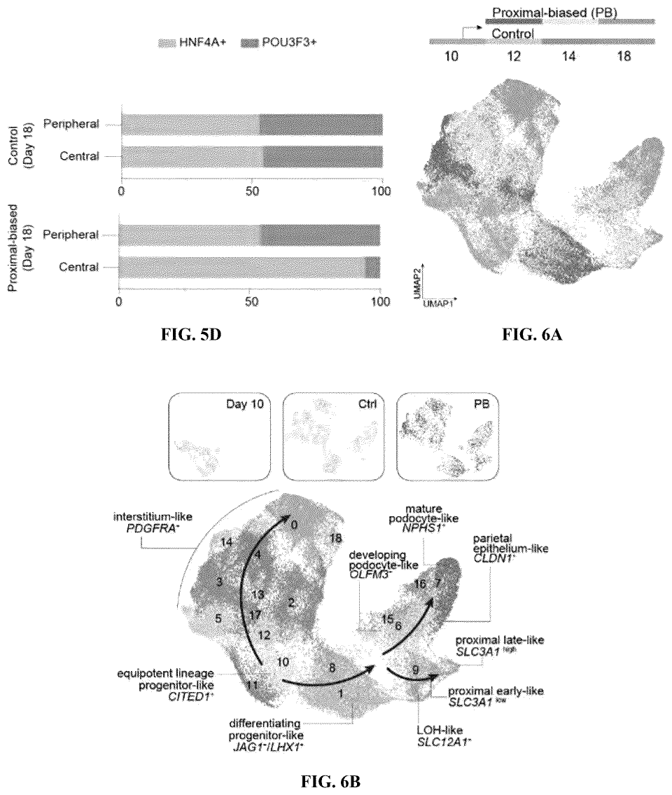

Upregulation of JAG1 and HNF1B occurred in nephrons alongside elongation (HNF1B 1.64-fold size increase, p-value <0.0001; JAG1, 2.36-fold size increase, p-value <0.0001) and the abundance of HNF1B was also increased within each nephron (1.22-fold increase, p-value <0.0001) compared to controls (FIGS. 4A-4B). These changes were dependent on Notch signaling as JAG1⁺/HNF1B⁺ nephrons did not elongate or further upregulate HNF1B when cultured with Notch antagonist DAPT, either with or without Ly29 (FIG. 5A). The protein encoded by Notch target gene HES1, a marker for Notch signaling, was reduced in organoids treated with both Ly29 and DAPT, but was upregulated in organoids treated with only Ly29 (FIG. 5A). The increase in HES1 on Ly29 treatment persisted through differentiation days 13 and 14; 1 and 2 days after the removal of the inhibitor. Transiently blocking PI3K signaling therefore drives Notch signaling and HES1 protein production throughout organoid nephrons, while control organoid nephrons exhibited low and non-uniform HES1 protein levels (FIG. 5B). Importantly, inhibition of Ly29 altered the structural dynamics of the organoids, leading to the emergence of JAG1⁺/HNF1B⁺ structures distributed across both the periphery and center of the organoid discs, while control organoids predominantly exhibited tubulogenesis biased towards the periphery (FIG. 4A, FIG. 5B).

In vivo, the JAG1⁺/HNF1B⁺ state transitions into HNF4A⁺ proximal cells (FIG. 1B) with HNF4G co-upregulated when the proximal precursor domain elongates (FIG. 4E). While control organoids show some HNF4A expression by differentiation day 14 (FIG. 1D), primarily at the periphery of organoids (FIG. 4D), Ly29-treated organoids upregulated HNF4A extensively throughout organoids (FIG. 4D, FIG. 4F). Treated organoids increased the total number of HNF4A⁺ segments per organoid (4.02-fold increase, p-value <0.05), the average size of individual HNF4A⁺ segments (1.30-fold increase, p-value <0.001), and therefore the sum total area of HNF4A⁺ organoid nephron segments (2.83-fold increase for n=3 organoids) (FIGS. 4G-4I). As the organoid nephrons matured, they further upregulated HNF4A/HNF4A (2.31-fold increase in transcript levels, p-value <0.05) as well as HNF4G/HNF4G (1.72-fold increase in transcript levels, p-value <0.05) (FIG. 4C, FIG. 4F). This phenotype was confirmed using a second inhibitor of PI3K signaling, GDC-0941, that differs structurally from Ly29 (data not shown). Notably, the PB organoids had unaltered WT1 RNA and protein levels, marking differentiating podocytes (FIGS. 4C-4D). The spreading of nephron forming events from periphery to center of each organoid upregulated HNF4A centrally (FIGS. 5C-5D). Those nephrons positioned at the periphery increased expression of HNF4A and transcription factor POU3F3, which is only expressed in segment 3 in the proximal tubule, and otherwise distally. The upregulation of POU3F3 was confirmed by bulk RNA-sequencing. These data show organoid nephrons can be biased to upregulate transcription factors driving early proximal tubule development.

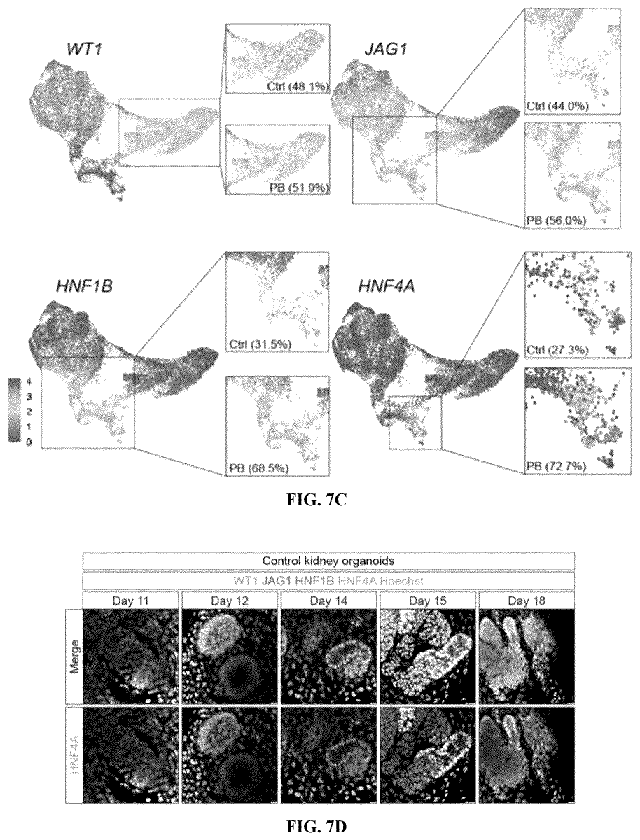

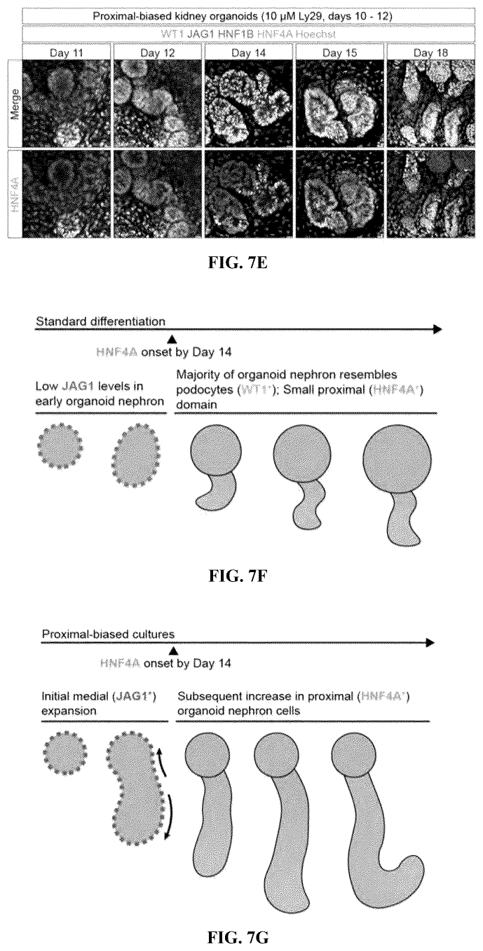

Enriching a functional proximal precursor identity in kidney organoids: To examine the process by which organoids develop PB cell identities in the protocol, control and Ly29-treated (PB) organoids were transcriptionally profiled using single-cell RNA-sequencing, sampling organoids across time (days 10 [untreated], 12, 14, and 18) and conditions. The organoid separated into nephron-like (PAX2⁺) and interstitial-like (PDGFRA⁺) cells (FIGS. 6A-6B). Day 10 organoid nephron cells showed WT1⁺/SIX1⁺/PAX2⁺ progenitor-like profiles (FIG. 6C). These cells were positive for CITED1 and robust expression of PAX8, indicating a lack of an in vivo nephron progenitor cells where PAX8 is normally detected at low levels in CITED1⁺ cells. Organoid nephrogenic cells separated by their sampling time, but overlap was observed between each time, thus forming an inferred developmental trajectory from progenitors through to HNF4A⁺ cells (FIGS. 7A-7C). The inferred trajectory suggests day 10 cells transition from progenitors and differentiate into nephron lineages by day 18 as evident from gradual changes in gene expression (FIGS. 6D-6F). These data validate the bulk RNA-sequencing and whole-mount immunofluorescent antibody staining, confirming increases in early proximal/medial gene expression such as HNF1B under PB conditions, and show elevated JAG1 precedes HNF1B and subsequent HNF4A expression (FIG. 7C). Control and PB organoid nephron cells co-clustered, indicating that early and transient Ly29 treatment coaxed cells towards a PB state, increased the number of these cells, but without eliciting other transcriptional changes that drive a transcriptional state sufficiently distinct to separate clusters. 72.7% of all HNF4A⁺ cells originated from the PB condition, with similar ratios in HNF1B and JAG1 (FIG. 7C).

To understand the cellular composition of the control and PB organoid nephron cells, the study integrated and compared their transcriptional profiles with in vivo nephron cells (FIG. 2A; FIG. 6G). Organoid nephrogenic cells mostly co-clustered with human nephron cells (FIG. 6H). Sample contributions for each cluster were quantified (FIGS. 6H-6I), revealing parallels and discrepancies between in vivo and in vitro-derived PB organoid nephron cells. Organoid podocyte (clusters 2 and 3: MAFB⁺, OLFM3⁺, NPHS2⁺), early proximal (cluster 12: HNF1B⁺, JAG1^{high}), proximal (cluster 10: HNF1B⁺, HNF4A⁺, SLC3A1+), and loop of Henle precursor (cluster 9: SLC12A1⁺, MAL⁺) cells co-clustered with in vivo cells. In contrast, human CITED1⁺ nephron progenitors (clusters 0 and 5) did not co-cluster with day 10 organoid cells (cluster 1) since CITED1⁺ day 10 organoid cells also express PAX8 and LHX1 (FIGS. 6G-6J). Distinct cluster were observed between day 12 control (clusters 7 and 8: PDGFRB⁺, PI3K^{HIGH}), day 12 PB (cluster 4: JAG1⁺, DLL1⁺, Notch^{HIGH}), and early human nephron (cluster 6: SNAI2⁺, DAPL1⁺) cells, pointing to transcriptional differences induced by Ly29 treatment (FIGS. 2J-2L, FIG. 6H). Distal human nephron cells (clusters 20, 19, and 16) separated from organoid nephron cells. These data collectively indicate that the PB organoids primarily generate podocyte and proximal cells, and few other cellular identities.

Validating these data in individual organoid nephrons showed consistent views (FIGS. 7D-7G). Morphologically, PB organoids diverge from controls, with a shift to HNF4A⁺ cells, leading to an expansion of the HNF1B⁺/HNF4A⁺ identity throughout the tubular portion of the organoid nephron. Initially, PB organoids display a subtle increase in the WT1⁺ domain immediately following PI3K inhibition at day 12 (1.08-fold larger, p-value <0.01) (FIGS. 6K-6L). However, this effect is not sustained and compared to control organoids, each control nephron displays large WT1⁺ renal corpuscle-like structures which are larger than those in the PB organoids (1.42-fold larger, p-value <0.001) (FIGS. 6K-6L, FIG. 7D, FIG. 7F). Instead, the PB organoid nephrons show an elongated HNF4A⁺ proximal domain (FIGS. 4F-4I, FIG. 7E, FIG. 7G) compared to controls (FIG. 7D, FIG. 7F).

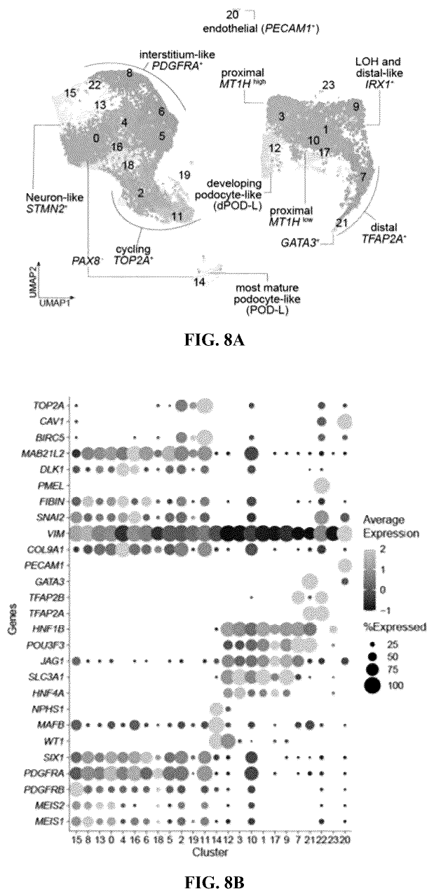

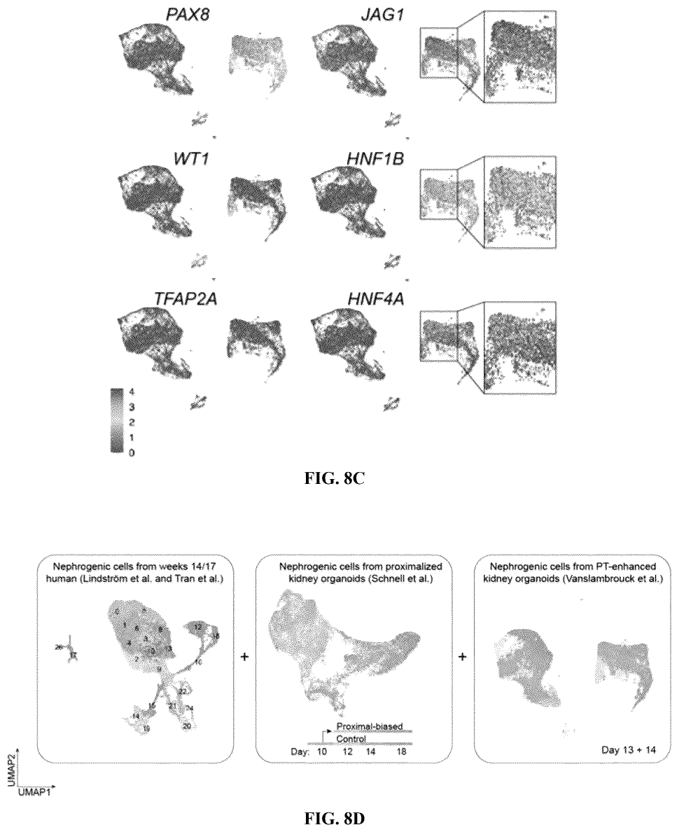

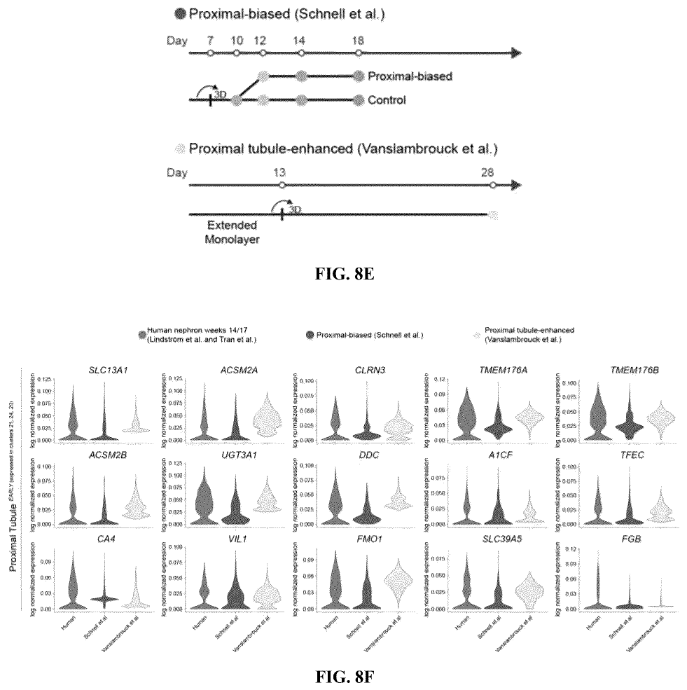

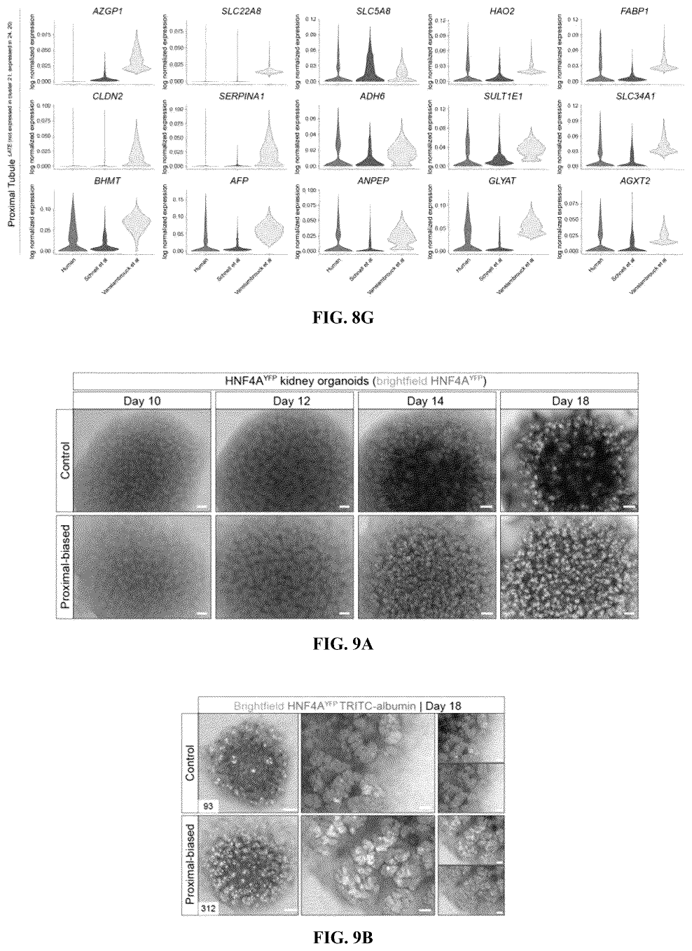

To scrutinize how PB nephrogenic cells compare against other kidney organoid proximal tubule models, the study performed a three-way analysis between the PB organoid nephrons (FIGS. 7A-7C), cells from a study generating proximal tubule-enhanced kidney organoids (FIGS. 8A-8C), and the study used the in vivo human proximal developmental program as a benchmark (FIGS. 2A-2C). Because cells from these data were captured using different single-cell technologies, the study mapped each organoid dataset to the in vivo reference using the Mutual Nearest Neighbor (MNN) method to mitigate batch effects (FIG. 8D). In addition, since organoids were sampled at discrete timepoints (FIG. 8E), while human in vivo data represents a continuum of differentiation, the study used the HNF1B and HNF4A-correlating genes to build a reference for the ‘early’ and ‘late’ proximal developmental programs (early: genes expressed in human clusters 21, 24, and 20; late: genes not expressed in human cluster 21, but expressed in clusters 24 and 20) (FIG. 3A).

Using these reference points, organoid cell profiles were assessed using the distribution of expression of early and late genes using a Wilcoxon Rank-sum test, incorporating a Bonferroni correction for multiple testing. Early proximal genes in PB organoid nephron cells closely resembled the distribution and expression levels detected in human cells (for instance ACSM2A, SLC39A5, UGT3A1) while proximal tubule-enhanced cells show a distinct grouped expression profile (FIG. 8F). Among the early proximal genes analyzed, 14/15 displayed an expression distribution statistically closer between the in vivo and PB organoid data compared to the proximal tubule-enhanced data (TABLE 5, TABLE 6). Similarly, late proximal tubule genes (for instance OAT3/SLC22A8, SLC34A1, SERPINA1) were expressed at lower levels in the in vivo and PB organoid cells compared to proximal tubule-enhanced organoid cells (FIG. 8G, TABLE 7, TABLE 8). A likely explanation for these differences is that proximal tubule-enhanced organoid nephron cells reflect a single late timepoint, while PB nephron and in vivo cells capture a differentiating proximal program including a series of proximal precursor states.

TABLE 5
Statistical analyses (Wilcoxon Rank-sum tests with Bonferroni correction for
multiple testing) to assess the degree of statistical difference in the expression
distributions from single-cell RNA-sequencing, separately comparing in vivo
weeks 14/17 human nephron cells to proximal-biased cells or proximal tubule-
enhanced cells in FIG. 8F. The analysis focused on 15 genes selected from FIG.
3A, representing early HNF4A/HNF1B correlating genes expressed in clusters 21,
24, and 20 of the human nephron single-cell RNA-sequencing dataset.

| | p value H v V | p value H v S | logFC H v V |
|---|---|---|---|
| SLC13A1 | 5.46707851616688e−22 | 0.01101196 | −0.2981481 |
| ACSM2A | 1.15335155728475e−104 | 0.616610302 | −0.8056453 |
| CLRN3 | 1.6358829427106e−16 | 3.43017293689279e−05 | −0.1278128 |
| TMEM176A | 2.68010672645973e−23 | 0.000350714 | −0.2930962 |
| TMEM176B | 3.04480078741674e−14 | 0.056794103 | −0.237503 |
| ACSM2B | 1.08074829740041e−74 | 1.15413716760713e−05 | −0.6524598 |
| UGT3A1 | 2.49677994468468e−25 | 2.45494538076594e−08 | −0.3116427 |
| DDC | 2.15915767473085e−61 | 0.423988518 | −0.5194341 |
| A1CF | 5.7544821033159e−34 | 6.50572751124716e−05 | −0.1037517 |
| TFEC | 8.72746331239872e−37 | 3.72842251129364e−17 | −0.3185589 |
| CA4 | 0.000555116 | 0.003552525 | 0.35918456 |
| VIL1 | 6.79581374832624e−20 | 0.000107968 | −0.2444852 |
| FMO1 | 8.62069805571195e−82 | 7.19428413267228e−08 | −0.6090224 |
| SLC39A5 | 4.25532726756223e−34 | 0.006439927 | −0.3838294 |
| FGB | 9.2756469412629e−58 | 8.28083444220484e−44 | 0.47610479 |

TABLE 6
Statistical analyses (Wilcoxon Rank-sum tests with Bonferroni correction for
multiple testing) to assess the degree of statistical difference in the expression
distributions from single-cell RNA-sequencing, separately comparing in vivo
weeks 14/17 human nephron cells to proximal-biased cells or proximal tubule-
enhanced cells in FIG. 8F. The analysis focused on 15 genes selected from FIG.
3A, representing early HNF4A/HNF1B correlating genes expressed in clusters 21,
24, and 20 of the human nephron single-cell RNA-sequencing dataset.

| | logFC H v S | adjusted p value H v V | adjusted p value H v S |
|---|---|---|---|
| SLC13A1 | 0.48728835 | 1.64012355485006e−18 | 1 |
| ACSM2A | 0.28320168 | 3.46005467185426e−101 | 1 |
| CLRN3 | 0.34432111 | 4.9076488281318e−13 | 0.102905188 |
| TMEM176A | 0.16897507 | 8.0403201793792e−20 | 1 |
| TMEM176B | 0.11686541 | 9.13440236225023e−11 | 1 |
| ACSM2B | 0.36578708 | 3.24224489220122e−71 | 0.034624115 |
| UGT3A1 | 0.45827376 | 7.49033983405403e−22 | 7.36483614229781e−05 |
| DDC | 0.41145113 | 6.47747302419254e−58 | 1 |
| A1CF | 0.01616377 | 1.72634463099477e−30 | 0.195171825 |
| TFEC | 0.1823851 | 2.61823899371962e−33 | 1.11852675338809e−13 |
| CA4 | 0.08201517 | 1 | 1 |
| VIL1 | −0.071398 | 2.03874412449787e−16 | 0.323904002 |
| FMO1 | 0.42726835 | 2.58620941671359e−78 | 0.000215829 |
| SLC39A5 | 0.3955241 | 1.27659818026867e−30 | 1 |
| FGB | 0.88959698 | 2.78269408237887e−54 | 2.48425033266145e−40 |

TABLE 7
Statistical analyses (Wilcoxon Rank-sum tests with Bonferroni correction
for multiple testing) to assess the degree of statistical differences
in the expression distributions from single-cell RNA-sequencing, separately
comparing in vivo weeks 14/17 human nephron cells to proximal-biased
cells or proximal tubule-enhanced cells in FIG. 8G. The analysis focused
on 15 genes selected from FIG. 3A, representing late HNF4A/HNF1B correlating
genes not expressed in cluster 21, but expressed in clusters 24 and 20
of the human nephron single-cell RNA-sequencing dataset.

| | p value H v V | p value H v S | logFC H v V |
|---|---|---|---|
| AZGP1 | 4.18995142313228e−154 | 2.71673437724698e−76 | −1.2052066 |
| SLC22A8 | 6.8706593612439e−142 | 0.182989037 | −0.790815 |
| SLC5A8 | 1.42263773860577e−45 | 2.85020740762631e−21 | −0.0228181 |
| HAO2 | 1.88722993317472e−74 | 9.52617398174495e−43 | −0.6085387 |
| FABP1 | 5.02127741116724e−94 | 3.37347323236298e−41 | −0.7195388 |
| CLDN2 | 1.99744985608808e−111 | 0.958391072 | −0.9194157 |
| SERPINA1 | 7.63358076509802e−75 | 3.59925625932857e−16 | −0.6805015 |
| ADH6 | 1.00851582927637e−47 | 2.71461230547046e−36 | −0.463531 |
| SULT1E1 | 1.12686754945549e−90 | 2.28356919145061e−35 | −0.7219843 |
| SLC34A1 | 4.03047687672695e−136 | 0.108680793 | −0.9895018 |
| BHMT | 3.37839195723254e−133 | 0.008096201 | −0.8670829 |
| AFP | 1.59803846530805e−143 | 1.31349989484515e−28 | −1.0015721 |
| ANPEP | 2.76894719907323e−90 | 0.399312867 | −0.6581221 |
| GLYAT | 4.79051747063005e−113 | 4.88896802398847e−07 | −0.8287407 |
| AGXT2 | 7.82302785090356e−75 | 0.457118867 | −0.5995072 |

TABLE 8
Statistical analyses (Wilcoxon Rank-sum tests with Bonferroni correction
for multiple testing) to assess the degree of statistical differences
in the expression distributions from single-cell RNA-sequencing, separately
comparing in vivo weeks 14/17 human nephron cells to proximal-biased
cells or proximal tubule-enhanced cells in FIG. 8G. The analysis focused
on 15 genes selected from FIG. 3A, representing late HNF4A/HNF1B correlating
genes not expressed in cluster 21, but expressed in clusters 24 and 20
of the human nephron single-cell RNA-sequencing dataset.

| | logFC H v S | adjusted p value H v V | adjusted p value H v S |
|---|---|---|---|
| AZGP1 | 0.76927115 | 1.25698542693969e−150 | 8.15020313174093e−73 |
| SLC22A8 | 0.91895254 | 2.06119780837317e−138 | 1 |
| SLC5A8 | −0.4086256 | 4.2679132158173e−42 | 8.55062222287894e−18 |
| HAO2 | 0.41616676 | 5.66168979952415e−71 | 2.85785219452348e−39 |
| FABP1 | 0.84472862 | 1.50638322335017e−90 | 1.01204196970889e−37 |
| CLDN2 | 0.96461227 | 5.99234956826425e−108 | 1 |
| SERPINA1 | 1.52599448 | 2.29007422952941e−71 | 1.07977687779857e−12 |
| ADH6 | 0.20336752 | 3.02554748782911e−44 | 8.14383691641139e−33 |
| SULT1E1 | 0.10135471 | 3.38060264836646e−87 | 6.85070757435184e−32 |
| SLC34A1 | 0.43288448 | 1.20914306301809e−132 | 1 |
| BHMT | 0.61018546 | 1.01351758716976e−129 | 1 |
| AFP | 0.69334163 | 4.79411539592415e−140 | 3.94049968453546e−25 |
| ANPEP | 0.62471676 | 8.30684159721969e−87 | 1 |
| GLYAT | 0.866441 | 1.43715524118902e−109 | 0.00146669 |
| AGXT2 | 0.34924038 | 2.34690835527107e−71 | 1 |

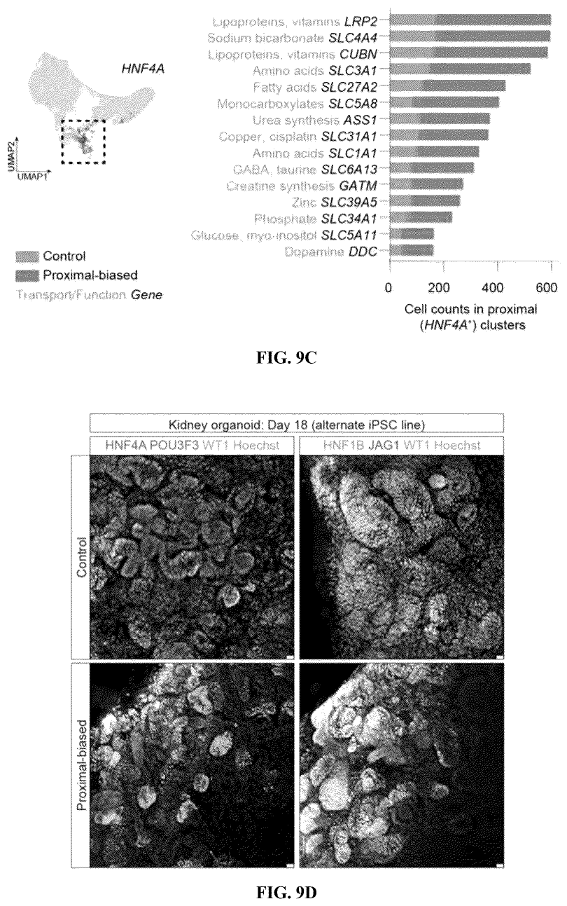

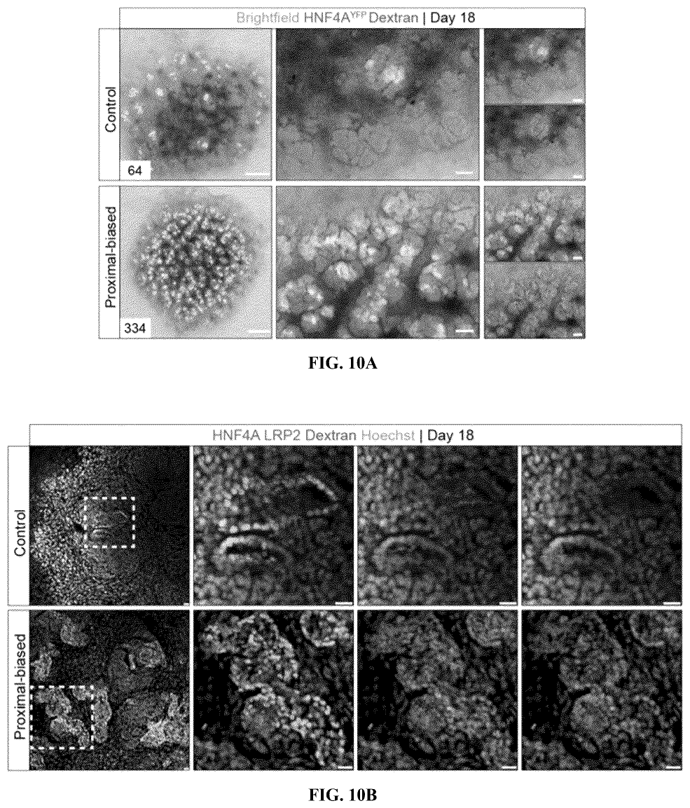

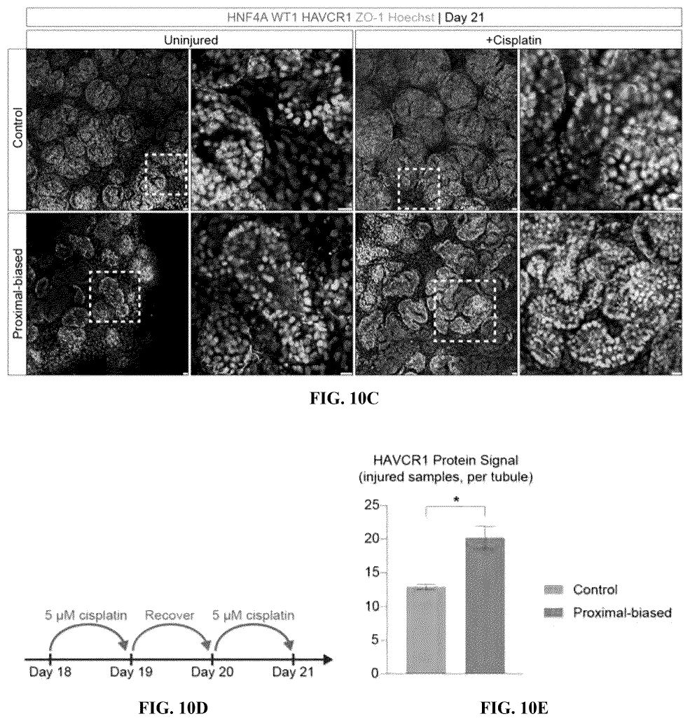

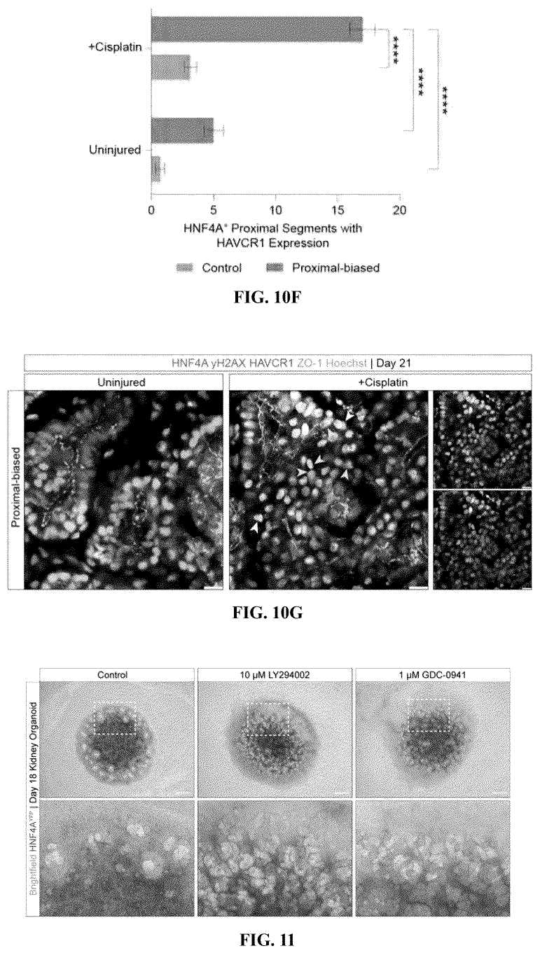

Modeling cisplatin-induced injury and proximal tubule physiologies in kidney organoids: Kidney organoid physiologies are often assessed by cells' ability to absorb and accumulate compounds that are known to be selectively transported by solute carriers in vivo. Uptake of dextran and albumin are associated with the function of HNF4A-target genes LRP2 and CUBN. PB nephrons display increased expression of LRP2 and CUBN compared to controls (LRP2: 1.32-fold higher TPM; CUBN: 1.48-fold higher TPM), raising the possibility that they exhibit proximal tubule functions. To assess this in live cells and to compare to previous kidney organoid models, the study used an HNF4A-YFP reporter iPSC line to label proximal tubule cells with yellow fluorescent protein (YFP), and tested whether PB kidney organoids display increased dextran and albumin uptake. Day 18 PB organoids showed strong HNF4A-reporter activity (FIG. 9A) and increased uptake of fluorescently tagged dextran (FIG. 10A; 6.87-fold increase, p-value <0.05) and albumin (FIG. 9B; 4.06-fold increase, p-value <0.05) compared to controls. Albumin and dextran were only detected within HNF4A-YFP⁺ regions of nephrons, and immunostaining post-assay confirmed that uptake was confined to HNF4A⁺, LRP2⁺ nephron segments highlighting proximal precursor-specific roles (FIG. 10B).

Single-cell RNA-sequencing data show PB cells express a broad range of genes associated with proximal tubule physiological functions, such as sodium bicarbonate transport (SLC4A4, 71.5% of cells from PB), monocarboxylate and iodide transport (SLC5A8, 79.0% of cells from PB), amino acid transport (SLC3A1, 71.8% of cells from PB), and copper transport (SLC31A1, 71.0% cells from PB) (FIG. 9C). SLC31A1 has also been shown to be important for uptake of cisplatin, a chemotherapeutic with severe nephrotoxic side-effects. Recent studies show that organoids are sensitive to cisplatin and respond by activating early proximal tubule injury marker KIM1/HAVCR1 expression and display evidence of DNA damage. To determine whether PB cells can model cisplatin-induced injury, PB organoids and controls were treated with two staggered low doses of cisplatin (5 μM) and assayed organoids 3 days after initial injury (FIG. 10D). Cisplatin treatment resulted in strong detection of KIM1/HAVCR1 in HNF4A⁺ PB nephron cells, while injured control organoids exhibited sparse KIM1/HAVCR1 (FIG. 10C). All HNF4A⁺ PB cells displayed KIM1/HAVCR1, but protein abundance was higher in PB organoid nephrons (FIG. 10E, 1.56-fold higher, p-value <0.05). Injured PB organoids contain significantly more KIM1/HAVCR1⁺ and HNF4A⁺ double-positive organoid nephrons compared to injured control organoids, as well as uninjured control and PB organoids (all p-values <0.0001) (FIG. 10F). Reports indicate that Hnf4a/HNF4A are downregulated in proximal tubules following injury and injury programs are mosaic. To determine whether the varied HNF4A protein levels in injured nephrons suggest different cellular responses, the study stained for DNA damage response marker γH2AX. Detection of γH2AX coincides with fluctuations in HNF4A protein levels, indicating a mosaic response (FIG. 10G). This suggests PB nephron cells provide a sensitive and rapid system to study cisplatin-induced cellular injury responses. Of note, interstitial γH2AX was detected in interstitial cells, suggesting broad DNA damage throughout organoids, requiring focused assays analyzing PB cells.

Given the improved capability of PB organoid nephrons to replicate physiological processes and display markers of injury, coupled with the reproducibility of biasing other iPSC lines (FIG. 9D), this approach emerges as a robust method for generating in vivo-mimicking proximal precursor cells within nephrons cultured in vitro.

### Discussion

This study developed informed approaches to mimic human proximal nephron precursor cells in organoids by delineating early in vivo human proximal tubule development, comparing it to existing kidney organoid models, and thereafter driving changes in cell states to bias differentiation outcomes. This model generates nephron-like structures in thin self-organizing discs that preserve nephron 3D complexities. This study takes advantage of the known plasticity in nephron positional identities and drive organoid nephrons towards a proximal-biased (PB) precursor-like state where organoid cells sequentially activate transcription factors and express HNF4A and function-imparting proximal tubule genes. This system offers a reproducible model to study development, origins of congenital disease, kidney injury, and physiology and is compatible with high-resolution microscopy. This approach is aligned with the broader body of work in kidney organoids, development, and disease modeling, and provides a targeted technique generating proximal tubule cell types with improved fidelity to their in vivo counterparts.

Benchmarking and recapitulating the proximal developmental program: This study demonstrates that in vivo, human proximal precursors develop through a series of cell-state transitions marked by activation of JAG1, followed by HNF1B, and eventually production of HNF4A in the upper bend of the medial S-shaped body nephron. Genetic evidence from mice shows that Notch signaling is required for proximal nephron differentiation, nephron segmentation, and nephron number. The emerging view is that the requirement for Notch signaling in proximal tubule development is deeply conserved between human and mice.

The study further shows that inhibition of PI3K results in a transient upregulation of Notch target genes, and upregulation of HNF1B. Inhibiting PI3K and simultaneously blocking Notch signaling in PB organoids prevents HNF1B expression, which is consistent with Notch1; Notch2 double knockout mice failing to upregulate Hnf1b in nephrons. In vivo, Hnf1b binds to and is required for expression of Hnf4a. A conserved role for HNF1B is indicated by mutations in Hnf1b/HNF1B being linked to congenital kidney anomalies affecting tubulogenesis, as well as studies in organoids. In this model, increased HNF1B expression and protein levels precede detection of HNF4A, concurring with in vivo and in vitro studies showing HNF4A is essential for proximal nephron cell-fate development.

While Hnf4a/HNF4A and Hnf4g/HNF4G are co-expressed in proximal tubules in vivo, evidence suggests a non-redundant relationship. Hnf4a knockout mice and HNF4A knockout kidney organoids have prominent phenotypes. Conversely, loss of HNF4G moderately alters kidney organoids, and Hnf4g knockout mice display a phenotype in the intestine only when removed alongside Hnf4a, due to the redundant roles of Hnf4 factors in enterocyte maturation. As part of comparative analysis between in vivo and in vitro, the study sought to understand how Hnf4a-dependent genes are expressed during development and in adult nephrons. It is interesting to note that a group of Hnf4a-dependent genes are not expressed in the adult, indicating temporally dynamic roles for Hnf4a, or that gene expression is mediated by indirect mechanisms requiring for example co-factors. Given the clear role of Hnf4a in injury and repair mechanisms, this is an area needing further scrutiny. Overall, this analysis points to that human orthologs of Hnf4a-dependent genes are expressed at much lower frequencies in organoids, which in turn suggests that organoids at present only partially mirror the in vivo proximal program. However, the onset of HNF4A and HNF4G expression occurs within a short timeframe in the S-shaped body nephron and PB organoids do express both genes as the nephron tubules elongate, which mirrors the temporal in vivo sequence. Other kidney models generating HNF4A⁺ proximal cell-states have previously enriched kidney organoid cultures for the nephron progenitor state and subsequently optimized culture conditions to permit proximal-like cell development. In contrast, the PB system imposes a specific signaling event switching cells to a proximal developmental program. For improving reproducibility, synchronicity, and application across cell-lines, increasing control over differentiation outcomes is an efficient strategy.

In all kidney organoid systems, it is unclear how JAG1 is initially activated in the forming nephron. In vivo, it is thought that Jag1 is bound and sensitive to β-catenin mediated transcription and Jag/JAG1 expression initiates in cells adjacent to the WNT9B secreting collecting duct. It is unclear how the brief dosing with CHIR at day 7 (a GSK3-β antagonist and WNT/β-catenin agonist) can control the emergence of JAG1⁺ nephron aggregates at day 10. Moreover, in vivo JAG1 expression follows an intriguing wave-like pattern where it moves proximally through the nephron and is downregulated distally, suggesting multiple layers of control over this Notch ligand. Identifying the mechanisms that regulate JAG1 expression and activities will be critical for understanding nephron proximal-distal patterning.

Modeling physiologies and injury in proximal-biased nephrons: In vivo, proximal tubule cells exhibit a well-defined apical-basal polarity, appropriately positioned solute carriers and transporters, vascularization, susceptibility to intraluminal solute flow, and sensitivity to nephrotoxic compounds. Modeling nephrotoxicity in PB organoids therefore lacks flow and vascularization, but they develop an apical-basal polarity and accumulate dextran and albumin in HNF4A⁺, LRP2⁺ cells. The study shows that PB nephrons are sensitive to Cisplatin early in the differentiation protocol (day 18), at a low concentration (5 μM), and within a quick timeframe (72 hrs), making the model suitable for screening experiments requiring short dosing plans and quick endpoints. Cisplatin is transported by a range of solute carriers, and bulk RNA-sequencing and single-cell RNA-sequencing confirm expression of several (maximum TPM values CTR1/SLC31A1: 120.92; OAT1/SLC22A6: 2.89; OAT3/SLC22A8: 3.83; OCT2/SLC22A2: 3.89). However, while detected, it remains to be determined whether cisplatin-induced damage requires active transport. Expression of HAVCR1/KIM1 was specific to HNF4A⁺ cells, but cisplatin-induced DNA damage was detected throughout organoids based on the DNA-damage marker γH2AX. Dissecting the mechanism by which HNF4A⁺ cells are injured by cisplatin would further validate its use as a proximal tubule nephrotoxicity model. Developing a tightly controlled synchronized injury model suitable for posing precise questions on the impact of compounds—such as chemotherapeutics, antibiotics, or other agents—is of high clinical value and would allow scrutiny of transition states that proximal nephron cells undergo post-injury.

The current PB model represents a cell-state that is best estimated to resemble a capillary loop stage based on the expression of some solute carriers. Generating a mature proximal tubule can include further modifications such as tuning of culture conditions to drive maturation, elongation, and separation of proximally distinct segments. Evidence shows that sorted proximal tubule-like cells from hiPSC-derived kidney organoids grown with or without immortalized cells can recapitulate proximal tubule physiologies and isolated proximal nephron tubules can be cultured for months, which introduces a direct possibility to adopt this protocol and generate PB organoids that are used as a cell source for long-term tubule culture systems. Organoid production can also be scaled to be suitable for large-scale screens, as shown in experiments using a bioreactor to produce kidney organoids. On their own, these organoids generate some LRP2⁺/Megalin⁺ structures, again raising the possibility of applying this approach to bias them to a proximal lineage. Beyond the manipulation of culture methods in static or dynamic systems, proximal nephron cells isolated from kidney organoids have been used in bioengineered chips to study pharmacological drug uptake. Chip technologies have further allowed for the introduction of nephrons with vasculature to model renal physiologies. These methods and the rapidly developing kidney organoid field contribute to the expanding toolkit available for kidney research and complement this developmental-mimicry approach.

### Example 2: Other PI3K Inhibitors

A study was conducted which tested an additional, structurally dissimilar inhibitor of PI3K signaling (GDC-0941) to confirm the effect from Ly29 on human iPSC-derived kidney organoids. Data are shown in FIG. 11.

GDC-0941 has slightly less efficacy than LY294002, which could potentially be attributed to their mechanisms of action. Ly29 inhibits PI3K activity via competitive inhibition of an ATP binding site on the p85α regulatory subunit of PI3K. GDC-0941 also competes for ATP binding, but on the catalytic subunit p110α. p110α is the catalytic subunit responsible for phosphorylating phosphatidylinositols, while p85α is the regulatory subunit that stabilizes and inhibits p110α.

### Example 3: Additional Injury Model Data

### Results

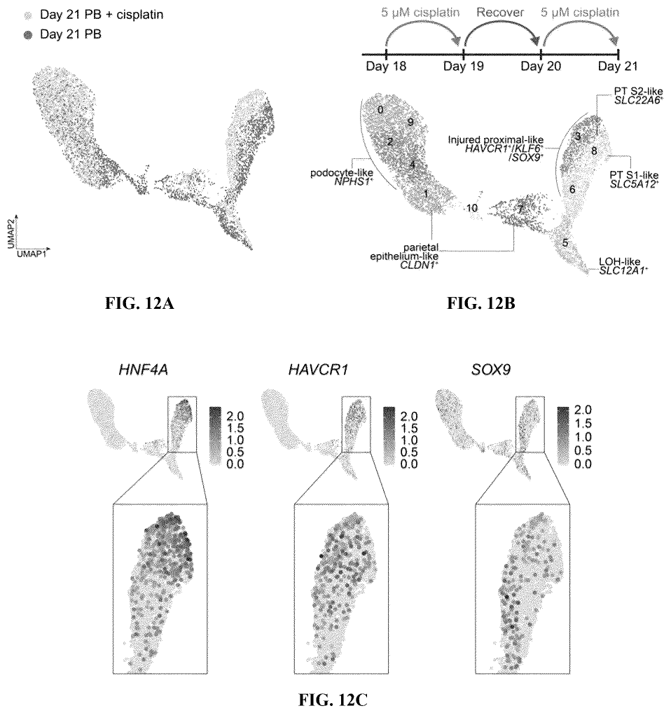

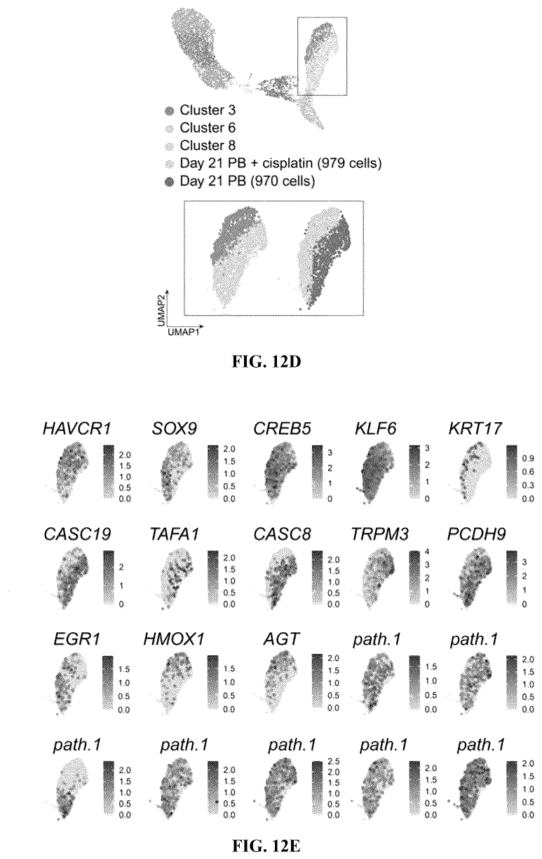

To evaluate how PB kidney organoids transcriptionally respond to cisplatin-induced injury, single-nucleus RNA sequencing on double-dose cisplatin-treated (injured) PB organoids was performed on day 21, and the data was merged with the single-nucleus RNA-sequencing data from uninjured PB organoids at the same time point (FIGS. 12A-12B). Podocytes co-clustered independent of injury condition, while tubule cells (HNF4A⁺ proximal-like, and SLC12A1⁺ loop of Henle-like) did not co-cluster and were separated based on injury status (FIGS. 12A-12B). HNF4A was readily detected in uninjured organoid nephron cells, while injured cells showed higher detection of HAVCR1 and SOX9, the latter showing a pronounced and injury-specific expression profile in the dataset that corroborated immunofluorescent data (FIG. 12C). Further examination of the proximal nephron subset revealed highly distinct clustering based on injury status (FIG. 12D), and injury-enriched genes (including SOX9 and KLF6, two markers of injured proximal tubules) were highly expressed in cisplatin-treated cells (FIG. 12E). Strikingly, KRT17 (keratin 17), a stress-responsive intermediate filament normally restricted to collecting duct epithelia but aberrantly expressed in states of kidney injury, regeneration, and tumorigenesis, was exclusively detected in the injured population (FIGS. 12E-12F). Similarly, injury-enriched and stress-response genes EGR1 (early growth response 1), HMOX1 (heme oxygenase 1), and AGT (angiotensinogen) were readily detected in the injured population. Percent contribution analyses validated that approximately 75% of all cells positive for these markers originated from the injured population (FIG. 12F), supporting the notion that these genes are indicative of a stress-induced or regenerative response specific to proximal tubule injury in PB kidney organoids.

Collectively, these data highlight the potential of the PB nephron proximal tubule cell model as a sensitive and rapid system to delineate nephrotoxic and acute kidney injury. Thus, to further elucidate the relevance of PB organoids as an injury and nephrotoxicity model, single-cell RNA sequencing data of the adult human kidney from the Kidney Precision Medicine Project (KPMP) was acquired and visualized (FIG. 12G). The dataset includes patient cells from three distinct disease categories of progressively worsening kidney disease: healthy reference, acute kidney injury (AKI), and chronic kidney disease (CKD). Focusing on the proximal tubule subset (FIG. 12G), the cells cluster into distinct groups corresponding to proximal tubule segments 1 and 2, segment 3, adaptive/maladaptive/repairing proximal tubule epithelial cells (aPT), and degenerative proximal tubule epithelial cells (dPT); cells from all three disease states were present across these four clusters (FIG. 12G), allowing for comprehensive analyses of gene expression across disease conditions. These findings were then compared to the gene expression patterns observed in single-nuclei RNA-sequencing of double-dose cisplatin-treated PB organoids, comparing both injured and uninjured cell states.

HAVCR1, SOX9, EGR1, and other injury-enriched genes (FIG. 12E) revealed a clear trend in the adult human kidney data: as disease progresses from healthy to AKI and further to CKD, the expression of these genes increases at higher frequencies (FIG. 12H—adult human kidney). A similar pattern emerged when comparing these adult human data to the PB organoid model: comparing uninjured to cisplatin-treated PB organoid cells, a nearly identical gene expression shift was observed (FIG. 12H—day 21 PB organoids), mirroring the progression seen in the human kidney data from healthy to degenerative states. This striking similarity suggests that the injury-induced response in PB organoid nephrons closely resembles that of the human kidney in pathological conditions, particularly the transition toward a more degenerated state (FIGS. 12G-12H). These results underscore the fidelity of PB organoids as an injury model that recapitulates human kidney injury responses. Proximal nephron cells in double-dose cisplatin-treated PB organoids are akin to those found in patients living with AKI and CKD. This direct comparison highlights the utility of PB organoids as a powerful tool for studying kidney injury and progression in a human-relevant model system. The PB organoid nephron, therefore, emerges as a robust method for generating in vivo-mimicking proximal tubule cells that replicate physiological processes and respond to injury-driving mechanisms.

### Discussion

Single-nuclei transcriptomic analyses provide a deeper understanding to the response of kidney organoid cells to injury. Specifically, the data suggested that injured and uninjured cells proximal cells do not co-cluster, and markers associated with injury (SOX9 and HAVCR1) were differentially expressed in the injured cells (FIGS. 12A-12H). This separation suggests that injury prompts substantial transcriptional reprogramming, reinforcing the notion that the injured state of proximal tubule cells is highly distinct, yet potentially reversible. Persistent expression of SOX9 in the injured cells is consistent with prior reports showing that Sox9 is linked to regenerative or fibrotic responses, adding further evidence that SOX9 is a potential mediator of injury-induced plasticity of proximal tubule cells. These injury-specific gene expression patterns, including the upregulation of KRT17, EGR1, HMOX1, AGT, and others (FIGS. 12E-12F) further highlights the molecular responses occurring in the injured cells: Egr1/EGR1 encodes a transcription factor rapidly induced by ischemic and toxic stress in proximal tubule cells in vivo, where it promotes a pro-regenerative program through activation of Sox9 and Wnt/β-catenin signaling; while Hmox1/HMOX1 is involved in the cytoprotective breakdown of heme and mediates oxidative stress responses and ferroptotic susceptibility in injured tubular epithelium. AGT is primarily synthesized in proximal tubule epithelial cells, where it serves as the local precursor of angiotensin peptides and reflects intrarenal renin-angiotensin system activation contributing to inflammation, glomerular hypertension, and fibrosis. The findings that these markers are upregulated in cisplatin-treated PB kidney organoids are consistent with the known role of these markers in acute kidney injury in vivo, where activation of pathways such as oxidative stress and ferroptosis susceptibility (Hmox1/HMOX1) and stress-induced transcriptional reprogramming that promotes SOX9-mediated epithelial repair (Egr1/EGR1) are key features of the injury response in kidney cells.

Remarkably, the PB organoid model demonstrates a high level of physiological relevance by recapitulating the same gene expression signatures and shifts that are observed in adult human kidney disease states: injury-enriched genes upregulated in cisplatin-treated PB organoids, including HAVCR1, SOX9, and EGR1, mirror the expression profiles seen in human proximal tubules as they progress from healthy to AKI and CKD (FIG. 12G; KPMP). This similarity underscores the potential of the PB organoid model to not only study the molecular mechanisms of kidney injury, but to also serve as a tool for developing therapeutic interventions aimed at enhancing the regenerative capacity of the kidney.

### Example 4: Stepwise Developmental Mimicry Generates Proximal-Biased Kidney Organoids with Emerging Proximal Tubule Cell Maturity

Proximal nephron cells are the most abundant cells in the human kidney, are responsible for reabsorbing 65% of the nephron filtrate, and their pathologies are the primary reason patients require dialysis and kidney transplants. In spite of their clinical importance, proximal tubule disease mechanisms are poorly understood, and human models are needed to scrutinize disease origins and etiology. Stem cell-derived human kidney models recapitulating proximal tubule functions would therefore provide a critical tool to study renal disease and develop new therapeutic approaches.

Directed differentiation protocols coaxing induced pluripotent stem cells (iPSCs) to intermediate mesoderm lineages have led to the development of human kidney-like organoids. These models partially replicate developmental kidney cell profiles, but do not form mature proximal tubule cells, and the proximal precursor-like cells that do develop exhibit low expression of genes that normally impart nephron-specific physiologies. Similarly, while current organoid models respond to nephrotoxic compounds by upregulating specific injury markers such as KIM1/HAVCR1 and γH2AX in LTL⁺ cells, the lack of homogenous proximal tubule like cells in organoids expressing nephrotoxin-transporting solute carriers limits their utility in studying acute proximal tubular injury and performing proximal nephron-specific drug screens.

Studies performed in mice and organoids show that proximal tubule development is dependent on expression of Hnf4a/HNF4A, whose transcription factor protein product is required for the normal expression of roughly 300 proximal tubule solute carriers, protein, ion, and substrate transporters, and other proximal nephron functional genes. Recently, effort has therefore focused on generating HNF4A⁺ proximal tubule precursors as these would serve as building blocks for generating functional proximal tubules. One strategy has enriched for this cell population by expanding the pool of nephron progenitors in early organoids and subsequently allowing their differentiation, but regardless of the protocol, proximal nephron precursors display relatively low expression of HNF4A and HNF4A-dependent genes.

In vivo, proximal tubule precursors emerge in the S-shaped body nephron following a stereotyped and deeply conserved developmental program. In this program, nephron progenitors are gradually recruited from their niche into pretubular aggregates that progressively undergo epithelial-to-mesenchymal transitions generating epithelial renal vesicles. Complex morphogenetic events form tubular Comma-shaped and thereafter the aforementioned S-shaped body (SSB) nephrons with distal and proximal gene signatures positioned along the emerging distal-to-proximal axial polarity. Transcriptionally distinct HNF4A⁺ proximal tubule precursors develop in narrow 2-3 cell-wide populations within each medial domain of SSBs, in a field of Notch ligand JAG1⁺ cells. Their development is dependent on Notch signaling, as Notch1 and Notch2 loss-of-function mice fail to normally express transcription factor Hnf1b, which in turn binds to and is necessary for Hnf4a expression. In addition to Notch, the development of the proximal-distal nephron axis requires integrated signaling between several pathways as spatial positions in the nascent nephron are known to be driven by Notch, Wnt, BMP, and PI3K signaling, each tuning the formation of precursor populations.

This study addresses the challenge of generating proximal tubule cells by developing a protocol to expand the HNF4A⁺ proximal nephron precursor population within kidney organoids and in individual nephrons. The study directs organoid cell differentiation along an in vivo-like developmental trajectory proceeding through JAG1⁺/HNF1B⁺ fates and culminating in maturing HNF4A⁺ proximal precursors. Comparative analyses with in vivo development show the organoid proximal precursors resemble HNF4A⁺ proximal tubule cells in the capillary loop stage (CLSN) nephron with emerging physiologies. Proximal-biased (PB) nephrons display expression of solute carriers and transporters and are capable of selectively transporting albumin and dextran. The study leverages the prevalence of proximal structures expressing cisplatin transporters to demonstrate significant upregulation of KIM1/HAVCR1 within HNF4A⁺ organoid nephron tubules in response to nephrotoxic injury. In line with in vivo injury, this response is mosaic and KIM1/HAVCR1⁺, HNF4A⁺ PB organoid nephrons display collapsed apical-basal polarities, DNA damage, and upregulate injury-response marker SOX9. The PB nephron model represents a significant step towards recapitulating kidney development and function in organoid systems and provides a direct strategy to study proximal nephrotoxicity and tubulopathies in a robust human assay.

### Methods

Human kidney samples: Developing human samples were collected under Institutional Review Board approved protocols (USC-HS-13-0399 and CHLA-14-2211). Following the patient decision for pregnancy termination, informed consent for donation of the products of conception for research purposes was obtained and samples collected without patient identifiers. The person obtaining informed consent was different than the physician performing the pregnancy termination procedure, and the decision to donate tissue did not impact the method of pregnancy termination. Developmental age was determined according to the American College of Obstetrics and Gynecology guidelines. The kidney samples ranged from 14 to 17 weeks of gestation with no sex reported. Intact samples within the kidney capsule were analyzed. Samples were transported on ice at 4° C. in high glucose DMEM (Gibco, 11965-118) supplemented with 10% fetal bovine serum (Genesee Scientific, 25-550) and 25 mM HEPES (Gibco, 15630080).

Human induced pluripotent stem cell lines: An iPSC line #3C15 FiPSC, derived from human male foreskin fibroblasts (BJ fibroblasts, ATCC, CRL-2522), was used. This line has been used in prior studies and protocols to generate human lung airway tissue, including induced pulmonary neuroendocrine cells. An HNF4A-YFP reporter iPSC line (male) was also used. An H1 hESC-derived iNPC line39 was used to make iNPC-derived kidney organoids following an established protocol.

iPSC Maintenance:

Matrigel-coated plate preparation: DMEM (Corning, 10-017-CV) was aliquoted into a 15 mL conical vial, and 120 μL of Matrigel (Corning, 354277) was added to make a 1% Matrigel mix. After thorough mixing, 2 mL of 1% Matrigel was pipetted into each well of a 6-well plate (or 1 mL/well for a 12-well plate). Matrigel plates were then incubated at 37° C./5% CO₂ overnight before use.

Biolaminin 521 LN-coated plate preparation: DMEM was aliquoted into a 15 mL conical vial and 300 μL of Biolaminin 521 LN (Biolamina, LN521) was added to make a 5% Biolaminin 521 LN mix. After thorough mixing, 0.5 mL of 5% Biolaminin 521 LN was pipetted into each well of a 12-well plate. Biolaminin 521 LN plates were then incubated at 4° C. overnight before use.

iPSC expansion and maintenance: iPSCs were thawed in Essential 8 media (Thermo Fisher Scientific, A1517001) on 1% Matrigel-coated plates. Media was initially supplemented with 10 μM Y-27632 (Rho kinase inhibitor from Tocris, 1254) for 24 hours after thawing iPSCs. Media was changed every 24 hours until cells reached 70-80% confluency (3 days). For freezing, when iPSCs reached 70-80% confluency, cells were collected for passage as described below and resuspended into a mix of 50% Essential 8, 40% KnockOut Serum Replacement (Thermo Fisher Scientific, 10828010), and 10% DMSO (Millipore Sigma, D5879). Cells were then aliquoted into 2 mL cryovials at 500,000 cells/vial, gradually frozen at −80° C. overnight, and then transferred to liquid nitrogen storage.

Directed differentiation to generate kidney organoids: Each biological replicate was generated from a distinct frozen vial of iPSCs. On reaching 70-80% confluency (3 days), iPSCs were rinsed with 1×PBS (Thermo Fisher Scientific, 10010049) and then incubated in TrypLE Select Enzyme (Thermo Fisher Scientific, 12563011) for 6 minutes at 37° C./5% CO2. The enzymatic reaction was neutralized using a volume of Essential 8 media 2× that of TrypLE. Cells were collected and resuspended in Essential 8 media supplemented with 10 μM Y-27632, with 15,000 cells/well plated onto a 5% biolaminin 521 LN-coated 12-well plate. 6 hours after plating, differentiation was initiated by changing culture media to TeSR-E6 (Stem Cell Technologies, 05946) supplemented with CHIR99021 (Tocris, 4423). Briefly, culture medium was supplemented with CHIR99021 for 5 days (exact concentration and duration are dependent on cell line being used), followed by 2 days with 200 ng/mL FGF9 (R&D Systems, 273-F9) and 1 μg/mL Heparin (Millipore Sigma, H4784). At day 7, the cells were detached using TrypLE as described above and resuspended in TeSR-E6 supplemented with 10 μM Y-27632. 200,000 cells were seeded into each well of a round-bottom non-adhesive 96-well plate. Organoids were manually transferred to a 0.4 μm pore culture plate (Corning, 3450; Stem Cell Technologies, 100-1026). Organoids received a pulse of TeSR-E6 supplemented with CHIR99021 for 1 hour; media was then switched back to TeSR-E6 supplemented with 200 ng/mL FGF9 and 1 μg/mL Heparin until day 12. Proximal-biased kidney organoids were cultured in TeSRE6 supplemented with 200 ng/mL FGF9, 1 μg/mL Heparin, and 10 μM LY294002 (Tocris, 1130) for 48 hours between differentiation days 10 and 12. Untreated control organoids were cultured with 200 ng/mL FGF9, 1 μg/mL Heparin, and DMSO vehicle. From day 12 onward, organoids were cultured in TeSR-E6 alone.

Small-molecule inhibitor screen and culture optimization: For all small molecule inhibitors used in this study, initial screens were performed on kidney organoids to determine the optimal dosage and duration of culture. To determine optimal dosage of a molecule, kidney organoids were cultured for 48 hours between differentiation days 10 and 12 in TeSR-E6 supplemented with the molecule at varying concentrations. After an ideal concentration was established, to determine optimal duration of treatment with the small molecule, separate differentiation experiments were set up with the small molecule from differentiation days 10 to 18, as well as differentiation days 12 to 18. For all optimization screens, time-course bright-field imaging was performed, and organoids were collected for whole-mount immunofluorescent analyses on differentiation day 18.

Single-cell preparation of kidney organoids: Approximately 6 organoids from multiple independent wells of both untreated and treated (48 hour culture with 10 μM LY294002 between days 10 and 12) conditions were collected at differentiation days 10, 12, 14, and 18 for single-cell RNA-sequencing, from separate differentiation batches. The organoids were dissociated using Accumax (Stem Cell Technologies, 07921) at 37° C./5% CO₂ for 35 minutes and pipetted twenty times with a P-1000 wide-bore pipette tip every 5 minutes. For FACS analyses, the same number of organoids from separate differentiations were dissociated using 10 mg/mL Native Bacillus licheniformis Protease (Creative BioMart, NATE-0633), 2.5 mg/mL collagenase type IV (Worthington Biochemical, LS004186), and 50 μg/mL DNase1 (Worthington Biochemical, LS002058) in 1×PBS−/−. FACS data were processed using flowCore (Ellis et al., 2024). Dissociations were neutralized by using a 2× volume of AutoMACS Running Buffer (Miltenyi Biotec, 130-091-221). Cells were pelleted by centrifugation at 4° C. for 3 minutes at 300×g. After resuspending the pellet in 2 mL of AutoMACS Running Buffer, cell suspensions were strained through 40 μm strainer (VWR, 21008-949), washing the filter with an additional 1 mL AutoMACS. The flow-through was pelleted, again at 300×g at 4° C. for 3 minutes. Cells were gently resuspended in AutoMACS Running Buffer supplemented with 14 μM DAPI (Thermo Fisher Scientific, D1306) and 5 μM DRAQ5 (Thermo Fisher Scientific, 62-254) and sorted on an ARIA I FACS at a low flow rate. Viable cells (DRAQ5-positive, DAPI-negative) were collected into a 1.7 mL low binding tube (Corning, 3207).

Single-nucleus preparation of kidney organoids: For single-nucleus RNA-sequencing, the same number of organoids from multiple independent wells were snap-frozen in liquid nitrogen before nuclei isolation. Briefly, organoids were transferred to a 1.7 mL low binding tube, centrifuged to form a pellet, and snap-frozen in liquid nitrogen. Flash-frozen organoids were dounced in cold Nuclei EZ Lysis Buffer (Sigma, N3408) supplemented with protease inhibitor (Roche, 05892791001). Homogenization was performed using a loose pestle and tight pestle system (Sigma, P0485) with 15 loose-dounce strokes, followed by filtration through a 200 μm strainer (pluriSelect, 43-50200). The homogenate was then subjected to an additional 5 strokes with the tight pestle and incubated on ice for 5 minutes. The suspension was filtered through a 40 μm strainer (pluriSelect, 43-50040) and centrifuged at 500×g for 5 minutes at 4° C. in a swinging-bucket centrifuge. After removing the supernatant, the nuclei pellet was resuspended in just Nuclei EZ Lysis Buffer, incubated on ice for an additional 5 minutes, and centrifuged again. The final nuclei pellet was resuspended in chilled 1× Nuclei Buffer (10× Genomics, #2000297) and filtered through a pre-wetted 10 μM filter (pluriSelect, 43-50010). All reagents contained Protector RNAse inhibitor (Promega, N2615) at a final concentration of 1 U/μL and DTT (Sigma, 6465663) at a final concentration of 1 mM.

Single-cell and single-nucleus RNA-sequencing data collection and analyses: SPLiT-seq single-cell processing and sequencing. Sorted, single, live cells were immediately processed and fixed using Evercode Cell Fixation kit (Parse Biosciences, ECF2001). Fixed cells were then stored at −80° C. until all samples were ready to be processed with the Evercode WT v2 single-cell RNA-sequencing platform (Parse Biosciences, ECW02030). After recovery of barcoded cells, cDNA was cleaned up and amplified by PCR, as per Evercode WT v2 platform protocols. cDNA quality was examined at multiple points on a 4200 TapeStation (Agilent) for yield and quality assessment. Paired-end sequencing was performed on the NovaSeq X Plus PE150 (Novogene).

Single-nucleus processing and sequencing: Single-nucleus RNA-sequencing libraries were generated using the 10× Genomics Chromium Next GEM Single Cell Multiome reagents (PN 1000283) following the manufacturer's protocols (10× Genomics; CG000338 Rev E). Briefly, freshly prepared single-nuclei suspensions were counted on a Countess 3 FL cell counter and approximately 9,000 nuclei per condition went through 1 hour of transposition at 37° C. followed by GEM generation and barcoding on a Chromium X machine with immediate subsequent RT reaction. cDNA was generated from 7 preAmp cycles and 8 cDNA cycles. GEX libraries were made from fragmented cDNA and 12 SI cycles. Sequencing was performed on a NovaSeqX 10B machine with 100 cycle Illumina flow cells at the Translational Genomics Center, Children's Hospital Los Angeles (CHLA) Center for Personalized Medicine.

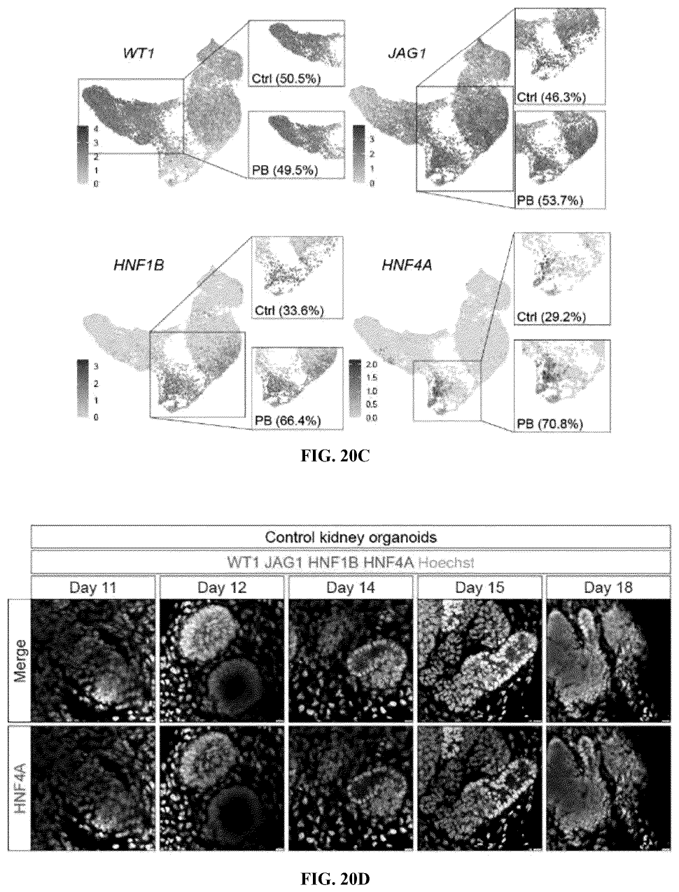

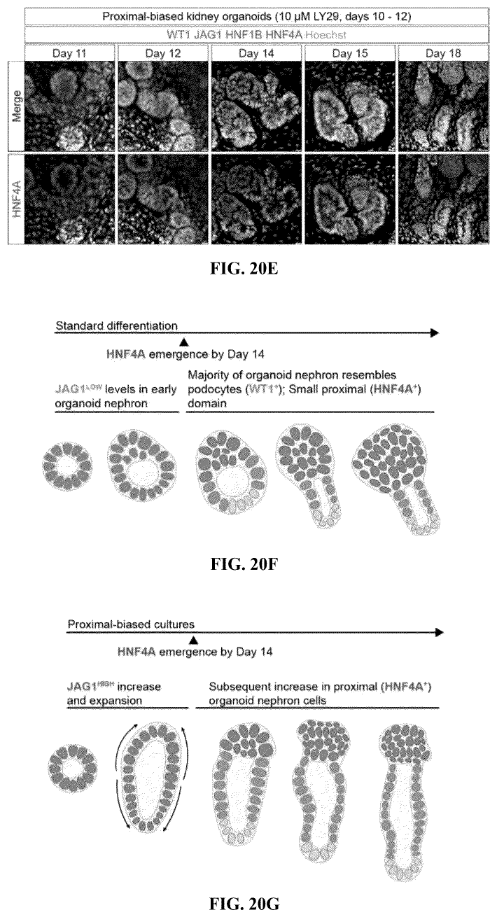

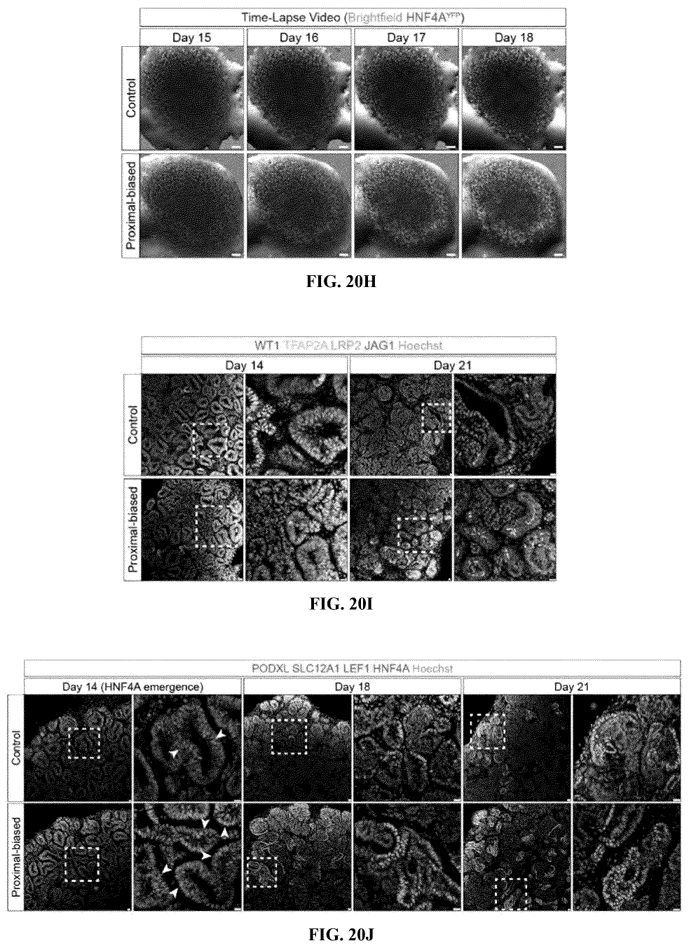

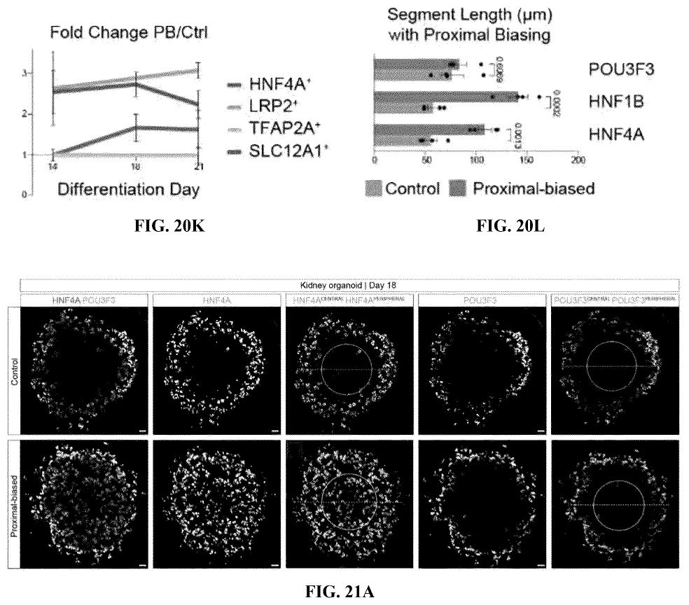

Single-cell RNA-sequencing analyses: From fastq files, demultiplexing, quality control, alignment to reference genome (hg38 Ensembl 105 annotation), and generation of count tables were done using splitpipe (Parse Biosciences). The Seurat 4.0 package was used for single-cell RNA-sequencing analyses. To filter out low-quality cells, the study kept cells with between 500 and 13000 features, between 100 and 200000 RNA counts, and less than 35% mitochondrial gene content. Sample datasets were integrated using Seurat integration functions across organoid time-points and experimental variables. 50 PCs were used to calculate cell relationships, cluster assignment, and UMAPs. After quality control, the organoid single-cell dataset included 9,686 cells from day 10, 22,229 cells from control samples (days 12, 14, and 18), and 24,405 cells from proximal-biased samples (days 12, 14, and 18). For the percentages shown in FIG. 20C, which represent the proportion of cells expressing each gene that originated from the control and proximal-biased samples, these values were scaled to normalize for differences in the total number of cells post-quality control between the control and proximal-biased samples. Differentially expressed genes of each cluster were identified using the FindAllMarkers function, selectively returning genes with expression in at least 25% of cells within the cluster (min.pct=0.25) and with a minimum fold-change of 0.25 (log fc.threshold=0.25), while restricting the output to only positively expressed markers (only.pos=TRUE). Nephrogenic cell-clusters were identified based on expression of nephron lineage and nephron cell-fate markers, and interstitial lineages similarly separated by their gene enrichment. Nephron-forming cells were selected by serial clustering and subsetting while monitoring cluster-based quality metrics.

Single-nucleus RNA-sequencing analyses: Single-nucleus RNA-sequencing data was processed using Cell Ranger (v9.0.0) and the Seurat 5.2 R package. Raw FASTQ files were processed using the cellranger count command, aligning to the pre-built human reference genome (GRCh38-2024-A). All minimum QC thresholds were achieved from sequencing with an average GEX saturation of 80% and greater than 3,000 median gene expression per nucleus. Feature-barcode matrices were subsequently imported into Seurat using the Read10× function. Quality control was performed in Seurat as described above. 30 PCs were used to calculate cell relationships, cluster alignment, and UMAPs. After quality control, the organoid single-nucleus dataset included 6265 cells from the day 21 proximal-biased sample, and 6637 cells from the day 27 proximal-biased sample. Differentially expressed genes for each cluster were identified, and the nephrogenic lineage was subset, each as described above.

In vivo kidney datasets: In vivo datasets of human week 1418 (GSE139280) and human week 178 (GSE124472) were analyzed independently (FIG. 2, FIG. 14) and were used for comparison to single cell and single-nucleus RNA-sequencing profiles of the nephrogenic subsets of differentiating organoids. To identify genes that are co-expressed with HNF1B and HNF4A, a Pearson correlation test was performed for each factor, and visualized against clusters 9 (JAG1⁺, HNF1BLOW, HNF4A−), 21 (JAG1⁺, HNF1B⁺, HNF4ALOW), 24, and 20 (JAG1LOW, HNF1B⁺, HNF4A⁺), which served as the early medial to proximalizing lineage (FIG. 2, FIG. 3, FIG. 14). The expression of these genes in vivo and in corresponding clusters of reanalyzed kidney organoid nephrons (FIG. 2, FIG. 14; assembled as described below) was analyzed in FIG. 3. Lastly, this human single cell RNA-sequencing dataset was integrated with the proximal-biased organoid dataset using RunCCA.

In vitro organoid datasets: To unbiasedly assess transcriptional profiles of proximalizing nephron cells, the study assembled and reanalyzed differentiating kidney organoids generated from multiple sources. Data from day 25 (GSE102596)6; days 7, 15, and 29 (GSE136314)7; days 16 and 28 (GSE124472)8; and day 26 (GSE118184)9 organoids were reanalyzed. Raw data from each dataset were acquired and analyzed individually as above before being integrated. This integrated dataset was subset into nephrogenic cell types for direct comparison to in vivo nephrogenic cells (FIG. 2, FIG. 14). Here, clusters 7, 6, 14, 12, 8, 18, and 3 served as the proximalizing lineage (FIG. 3). Separately, the study re-analyzed published single-cell RNA-sequencing data from organoids from a protocol generating proximal tubule-enhanced kidney organoids (GSE184928). These data were acquired and re-analyzed independently.

Integration and batch effect correction: Single-nucleus RNA-sequencing data from day 21 proximal-biased and day 27 proximal-biased kidney organoids were merged and integrated with the single-cell RNA-sequencing dataset from days 13⁺14 (27) proximal tubule-enhanced organoids 10 using sctransform to mitigate batch effects and integrated using IntegrateLayers with the canonical correlation analysis (CCAIntegration) method in Seurat 5.2.

Pseudotime reconstruction of lineages: Podocytes were subset from the organoid nephron single-cell RNA-sequencing dataset for a more streamlined developmental trajectory analysis from a day 10 equipotent lineage progenitor cell to a day 18 proximal-biased nephron cell. Monocle 3 was used to reconstruct the differentiation trajectory.

Gene expression heatmaps: Gene expression heatmaps comparing organoids at individual timepoints were generated using the AggregateExpression function in Seurat 5.2 on the list of all differentially expressed genes identified in the dataset. DEGs were calculated as described above and by setting the cluster identifier to the sample, enabling direct comparison of samples rather than cluster numbers. For the heatmap displaying three samples (day 10, day 12 control, and day 12 proximal-biased), Z-scores were calculated from the aggregated expression values and visualized using the pheatmap R package. For the remaining heatmaps, where only two samples were compared at each timepoint (control vs. proximal-biased), aggregated expression values were used to calculate log₂ fold changes (log₂FC) of the proximal-biased sample relative to the control. These log₂FC values were subsequently ordered and plotted as heatmaps using pheatmap.

Other datasets referenced: To analyze Hnf4a-dependent gene expression in kidney organoids, the study acquired bulk RNA-sequencing data from control and Hnf4a-mutant P0 mouse kidneys (GSE144772). Bulk RNA-sequencing fastq files from two control and two mutant (Osr2Credriven deletion of Hnf4a) P0 kidneys were acquired. Sample quality was assessed using fastqc. Using STAR version 2.7.10a, reads were aligned to the mouse mm10 genome, Ensembl release 102. Mapping quality was appraised using qualimap. Resulting .bam files were then used to generate a feature counts table using featurecounts. A list of 257 genes that are significantly downregulated in the Hnf4a-mutant kidney (DESeq2 comparing control vs. mutant kidneys, padj<0.01, log₂FC≥1.5) were used for analysis (FIG. 3). Separately, a list of 415 genes that are highly expressed and enriched in the adult mouse proximal tubule (GSE129798; Kidney Cell Explorer) were used for analyses in FIG. 3. To generate the heatmaps presented in FIG. 3, three proximalizing human clusters (FIG. 2, FIG. 14, clusters 20, 21, and 24) were analyzed, alongside the organoid data, which exclusively focused on cluster 3. These clusters were selected based on their relatively elevated expression levels of HNF4A, each surpassing 15% of all cells in the cluster. Human orthologs of mouse genes were retrieved using Ensembl BioMart, and genes expressed in less than three cells of the human dataset were removed.

mRNA-sequencing and data analyses: Whole organoid samples (3 independent biological replicates per timepoint, per condition for differentiation days 10 and 12, and 2 replicates for differentiation days 14 and 18) were prepared and purified according to the RNeasy Mini Kit (Qiagen, 74104). Purified RNA was sent to Novogene for sequencing. Reads were aligned to the human genome hg38 Ensembl 105 annotation using STAR version 2.7.10a. A noise-reduction filter was applied to keep genes in which the maximum count in at least one sample was greater than or equal to 10. Partek Flow version 10.0 was then used to normalize these raw feature counts following the transcripts per million (TPM) normalization method. After filtering genes with TPM values greater than or equal to 25 in at least one sample, Z-score averages were calculated and heatmaps were clustered using pheatmap (Klode, 2018). Separately, gene set enrichment analyses were performed specifically on Day 12 replicate samples by first normalizing noise-reduced feature counts using the DESeq2 median ratio method, as recommended for gene set enrichment analyses. Partek Flow version 10.0 was then subsequently used for gene set enrichment analyses.

### Immunofluorescent Imaging and Analyses:

Kidney organoids: Whole kidney organoids were fixed for 20 min on ice in 4% PFA (Electron Microscopy Sciences, 15710) in 1×PBS. In 1×PBS, organoids were then carefully cut out of the permeable membrane well and placed into 1×PBS with 1.5% SEA Block (Thermo Fisher Scientific, 37527X3) and 0.1% TritonX100 (EMD Millipore, 1.08643) (blocking solution) at 4° C. with gentle movement for one hour. Primary antibodies were resuspended in blocking solution, and organoids were incubated in primary antibody and blocking solution overnight. Samples were rinsed and then washed for at least 3 hours through several rounds of 1×PBS with 0.1% TritonX100. Secondary antibodies were resuspended in blocking solution, and organoids were incubated in secondary antibody and blocking solution overnight. Rinsing and washing steps were repeated the next day before a 25 minute counterstain incubation in 1×PBS supplemented with 0.1% TritonX100 and 1 μg/mL Hoechst 33342 (Thermo Fisher Scientific, H3570) to stain nuclei. Organoids were then washed for 1 hour in 1×PBS alone. Individual organoids were mounted for imaging on slides with a glass coverslip in Immu-Mount (Thermo Fisher Scientific, 9990402). Slides were stored at 4° C. in the dark. Primary antibodies used in this study were: WT1 (abeam, ab89901, 1:1000), JAG1 (R&D Systems, AF599, 1:300), HNF1B (Thermo Fisher Scientific, MA5—24605, 1:500), HNF4A (R&D Systems, MAB4605, 1:200), CDH1 (BD Biosciences, 610181, 1:300), ZO-1 (Thermo Fisher Scientific, 33-9100, 1:200), PAX2 (R&D Systems, AF3364, 1:50), SIX1 (Cell Signaling Technology, 12891S, 1:300), HES1 (Cell Signaling Technology, 11988, 1:300), POU3F3 (Novus Biologicals, NBP1-49872, 1:500), HNF4G (Thermo Fisher Scientific, PA5-82189, 1:200), LRP2 (My Bio Source, MBS690201, 1:500), HAVCR1 (R&D Systems, AF1750, 1:200), 7H2AX (Cell Signaling Technology, 2577), LAMB1 (Santa Cruz Biotechnology, sc-33709, 1:250), ATP1A1 (Abcam, ab7671, 1:200), SOX9 (Abcam, ab185230, 1:300), PODXL (R&D Systems, AF1658, 1:200), PAX8 (Abcam, ab189249, 1:100), LEF1 (Santa Cruz Biotechnology, sc-374412, 1:200), LTL (Vector Laboratories, B-1325-2, 1:300), SLC12A1 (Sigma Aldrich, HPA018107, 1:200), TFAP2A (Santa Cruz Biotechnology, sc-12726, 1:200), Alexa 647-conjugated LRP2 (R&D Systems, FAB9578R, 1:100), and Alexa 594-conjugated LRP2 (R&D Systems, FAB9578T, 1:100). Secondary antibodies conjugated with AlexaFluor 488, 555, 594, and 647 were all diluted to 1:500 in blocking solution.

Human kidney sections: Human kidneys were carefully dissected out from donated tissues and placed in 1×PBS. Kidneys were then placed into 4% PFA to fix overnight (18 hours). Kidneys were washed twice in 1×PBS (1 hour per wash), and samples were placed in 30% sucrose in 1×PBS rocking at 4° C. overnight (18 hours). The next day, kidneys were swirled in Optimal Cutting Temperature (OCT from Sakura Finetech USA Inc, 4583) compound 3 times before embedding in an OCT block. The OCT blocks with tissues were frozen on a dry ice/ethanol slurry. Blocks were stored at −80° C., and ˜12 μm sections were obtained using a cryostat. Slides were stored at −80° C. before being processed for immunofluorescent imaging.

Image acquisition and analyses: Image acquisition of whole-mounted organoids and cryosectioned human kidneys were performed using (1) Leica SP8-X confocal fluorescence imaging system (Leica Microsystems, Germany), in 1024×1024 pixels using 25× water, 40× oil, and 63× oil objectives, and (2) Leica Stellaris confocal microscope at 20× oil and 93× glycerol objectives. A Leica Thunder microscope was additionally used at 10× dry objective to image entire wholemounted kidney organoids. Live organoids were captured using (1) Zeiss Axio Zoom at 16×, 32×, and 112× objectives and (2) Olympus APX100 at 4× objective. Image masking was performed and quantified with Imaris 9.7 software (Oxford Instruments) and with Fiji. Fiji was used for individual cell and nephron segment counting, and for quantifying relative protein signal and organoid nephron size.

In vitro albumin and dextran uptake assays: For the albumin uptake assay, differentiation day 21 control and proximal-biased organoids were incubated at 37° C./5% CO₂ overnight (18 hours) in TeSR-E6 supplemented with 50 μg/mL TRITCalbumin (Sigma Aldrich, A2289) and Alexa 647-conjugated LRP2 antibody (R&D Systems, FAB9578R, 1:100). After the incubation, organoids were washed at least 3 times with 1×PBS and live imaged immediately after. For the dextran uptake assay, organoids were treated and processed in the same way, except that TeSR-E6 was supplemented with 20 μg/mL Alexa 647-conjugated Dextran (Thermo Fisher Scientific, D22914) and Alexa 594-conjugated LRP2 (R&D Systems, FAB9578T, 1:100).

In vitro cisplatin injury assay: For the kidney organoid cisplatin injury assay, the study adapted an approach undertaken by two recent studies. Differentiation day 18 control and proximal-biased organoids were incubated overnight (18 hours) at 37° C./5% CO₂ in TeSR-E6 supplemented with 5 μM cisplatin (Millipore Sigma, 232120). Organoids recovered between days 19 and 20, before another overnight culture (18 hours) between days 20 and 21 with 5 μM cisplatin. Uninjured organoids were cultured with DMSO vehicle. Organoids were then processed for immunofluorescent imaging and analyses.

### Results

Identifying abnormal kidney organoid developmental programs: To identify differences between developing nephrons in kidneys and organoids that can explain why organoids do not generate maturing proximal tubule cells, the study characterized how proximal precursors form in vivo in developing human kidneys and compared organoids to this framework using single-cell RNA-sequencing and secondary validation for proteins that mark and drive proximal tubule development.

The specification of proximal precursors is consequent to the gradual recruitment of nephron progenitor cells into the forming nephron and signaling pathways that tune differentiation along the progressively emerging proximal-distal axis. This process begins with a cellular domain developing in the distal renal vesicle nephron formed by early recruited nephron progenitor cells. The domain is marked by membrane-localized JAG1 and nuclear HNF1B and is in direct contact with the ureteric epithelium. It abuts the proximally located WT1⁺ region where cells are actively recruited from the nephron progenitor cell niche (FIG. 1A). As the renal vesicle develops into a comma-shaped body nephron, the HNF1B⁺/JAG1⁺ domain expands proximally. The initial distal domain downregulates JAG1 to form HNF1B⁺/JAG1LOW cells, while the proximally expanding domain further upregulates JAG1 to become HNF1B⁺/JAG1HIGH (now considered the medial domain of the comma-shaped body), and the medial domain forms a boundary with the WT1⁺ proximal-most domain where the last nephron progenitors are recruited (FIGS. 1A-1B). At the comma-shaped nephron stage, the nephron is subdivided by these markers into three distinguishable domains. As the nephron develops further, HNF1B is upregulated in nuclei positioned at the border between the medial and proximal-most domain, where HNF4A is detected in late comma-shaped/early S-shaped body nephrons (FIG. 1B). Proximal tubule precursor cells (HNF1BHIGH/HNF4A⁺) then develop and downregulate JAG1 when the S-shaped nephron matures into capillary loop stage nephrons adopting an elongated form with a HNF1BHIGH/HNF4AHIGH/JAG1− proximal tubule cell state (FIG. 1B). These findings are consistent with single-cell RNA-sequencing of the developing human nephron lineage (FIGS. 2AA-2C). Proximal tubule precursor cells diverge from other nephron lineages in an early PAX8⁺ cell population in the pretubular aggregate, which generates WT1⁺ podocyte precursors, TFAP2A⁺ distal precursors, and JAG1⁺ cells that sequentially upregulate HNF1B and HNF4A (FIGS. 2A-2C).

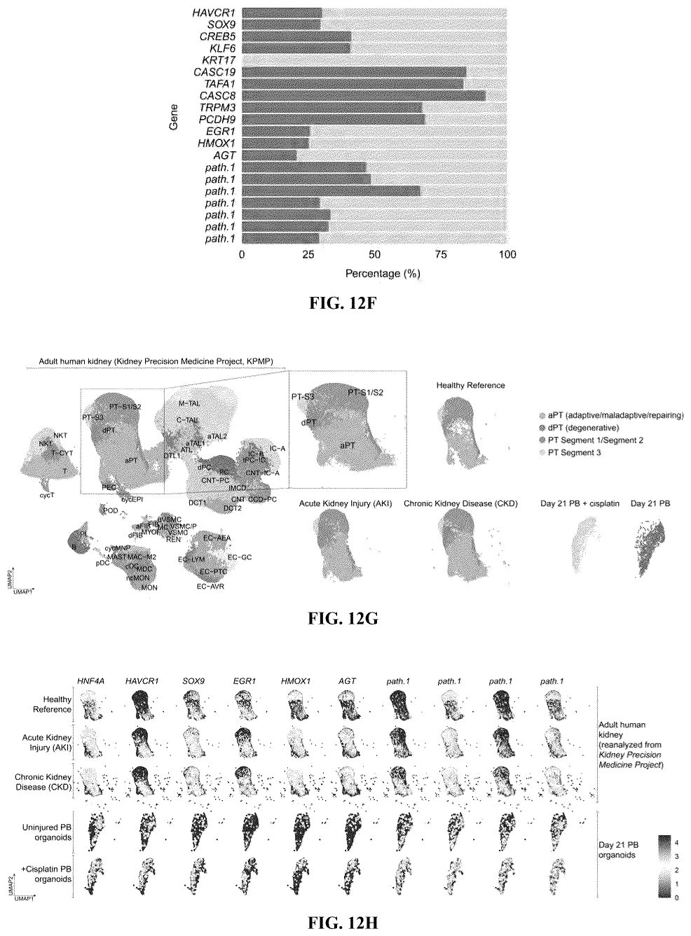

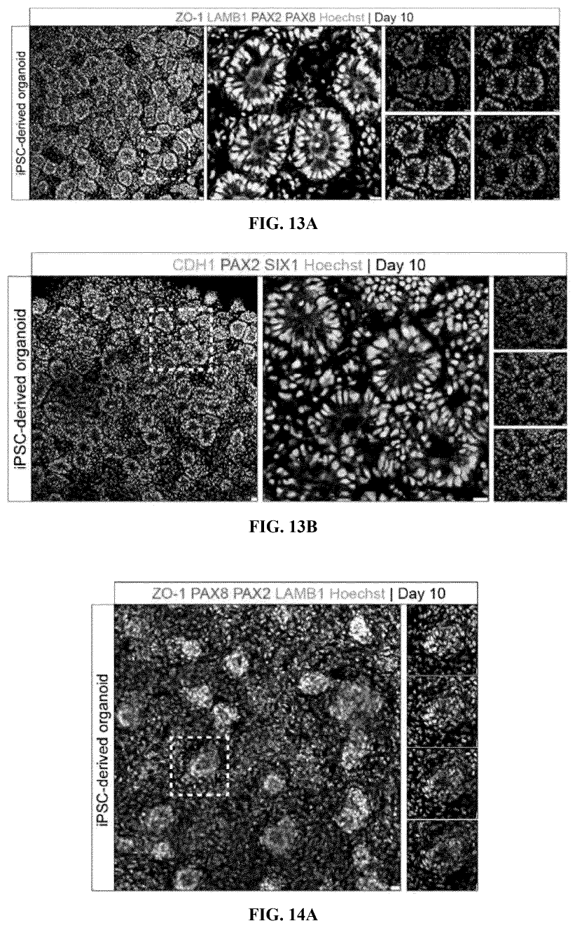

Organoid nephrons however, form differently. At day 10, kidney organoids include individual cell-aggregates positive for nephron progenitor markers (WT1, SIX1) and nephron lineage markers (PAX2, PAX8). These cells coalesce around a forming apical epithelial polarity (CDH1, ZO1, JAG1) with an accreting basement membrane (LAMB1). These dynamics are consistent across organoids generated from different human iPSC lines using a modified Takasato protocol (FIGS. 13A-13B, FIGS. 14A-14C).

As the aggregates further epithelialize (days 11-12), they display uniform deposition of WT1 in nuclei, and JAG1 as cytoplasmic puncta and weakly at cell membranes around the periphery of the forming nephron (FIGS. 1C-1D), while HNF1B is upregulated between days 11 and 12. At these stages, there are no indications of WT1, JAG1, and HNF1B being distributed along a gradually forming proximal-distal axis as seen in vivo (FIG. 1A), rather the organoid nephrons exhibit an initially homogenous HNF1B⁺/JAG1⁺/WT1⁺ triple-positive cell state (FIG. 1C). During further differentiation, organoid nephrons generate HNF1B⁺/HNF4A⁺ cells, but these do not transition into a rapidly elongating phase as observed in vivo and JAG1 remains strongly detectable (FIG. 1B, FIG. 1D). Single-cell data from organoids sampled over time and differentiation protocols indicate that the abnormal early and late triple-positive PAX8⁺/JAG1⁺/WT1⁺ and late HNF1B⁺/HNF4A⁺/JAG1⁺ cell states are a common trend across models (FIGS. 2D-2F).

Given the importance of Hnf1b and Hnf4a for normal proximal tubule development, the study examined the possibility that organoids abnormally regulate functionally important Hnf1b/HNF1B and Hnf4a/HNF4A-mediated transcriptional programs and assessed organoid proximal fate development against the in vivo developmental blueprint. To identify genes that are co-expressed with each transcription factor in vivo (FIG. 2A), a Pearson expression correlation analysis was performed for each gene and the expression of HNF1B and HNF4A correlates was characterized. The expression of these genes in vivo was ordered along a predicted proximal developmental trajectory, and their expression patterns highlight a transition from HNF1B correlates in early cells (cluster 9: pretubular aggregate/renal vesicle; e.g., JAG1, HES1, KRT8) through to a gradually more HNF4A-correlating signature (clusters 21, 24: S-shaped body; e.g., CLU, DCDC2, ANXA4), and finally genes strongly enriched in the maturing proximal precursors (cluster 20: Capillary loop stage nephron; e.g., ASS1, SLC34A1, SLC22A8; FIG. 3A (human)).

Using this progression as a framework for the temporal in vivo sequence of transcriptional events, the study compared it to organoid proximal precursor development across models (FIG. 3A (organoid) based on FIG. 2D). In vitro, HNF1B-correlates such as JAG1, HES1, KIF12, and KRT8 were detected while other genes, for instance transcription factor ELF3, fibroblast growth factor receptor FGFR4, serine protease inhibitor SERPINF2, and extracellular signaling protein CYR61 were not. This partial recapitulation of the transcription profile was further reduced for HNF4A-correlates, with only 25/46 genes detected across in vitro models. Genes coding for a range of protein types were not detected or significantly expressed, for instance solute carriers SLC22A8, SLC5A8, SLC16A9, and transferases and enzymes such as AGXT2, GLYAT, and ANPEP.

To independently examine whether HNF4A-dependent genes are underrepresented in the organoid proximal program, the study intersected genes downregulated upon in vivo Hnf4a loss-of-function, with transcriptional profiles from detailed expression maps of the adult male and female mouse kidney. The study categorized Hnf4a-dependent genes as those expressed only in development and those that persist into the adult functional nephron. 257 genes are significantly downregulated (control vs. mutant kidneys, padj<0.01, log₂FC≥1.5) in postnatal day 0 animals on loss of Hnf4a, and 415 genes are enriched in the adult male and female proximal tubules (FIG. 3B). Of the 257 Hnf4a-dependent genes, 124 genes were enriched only in the developing kidney while 133 genes showed persistent expression into functional adult nephrons. To determine whether human orthologs are expressed in the developing human proximal tubule and in organoid models, the study intersected the gene lists with single-cell transcriptional data (FIG. 2A, FIG. 2D). Of the 133 Hnf4a-dependent genes expressed during development and in adult mouse nephrons, 92 human orthologs were detected. 91/92 were also detected in organoids, and 89/92 (96.7%) were detected at greater abundance in vivo (FIG. 3C, FIG. 3E). Similarly, of the 124 Hnf4a-dependent genes expressed during development, 81 human orthologs were detected. 80 of these were detected in organoids, but 70/81 (86.4%) were again detected at a higher frequency in vivo (FIG. 3D, FIG. 3F).

These data show that correlates of HNF4A and human orthologs of Hnf4a-dependent genes are expressed infrequently in organoids. This is consistent across organoid models, collectively pointing to abnormal regulation of HNF4A-mediated gene expression.

Initiating a proximal-forming cell-state in kidney organoids: There exists a relationship between PI3K signaling and Notch ligand Jag1. In mouse kidneys, WNT/β-catenin and PI3K signaling have opposing effects on Jag1 expression, and pharmacological inhibition of PI3K signaling results in rapid upregulation of Jag1 in nephron progenitors and early nephrons. The study therefore tested whether inhibition of PI3K and upregulation of JAG1 could drive a proximal nephron program and provide a tool to generate human proximal tubule precursors in kidney organoids.

The study treated organoids with PI3K inhibitor LY294002 (Ly29) from days 10-12, when JAG1 is normally partially upregulated in vitro and organoid nephrons epithelialize. The study transcriptionally profiled them before and after treatment using RNA-sequencing. PI3K inhibitor-treated organoids displayed a 1.78-fold increase in JAG1 expression, and Notch pathway genes LNFG, HES1, and HES4 were upregulated, as were differentiation markers PAX8 and LHX1. Nephron progenitor markers SIX1, MEOX1, OSR1, and EYA1 were downregulated. Wnt/β-catenin targets WNT4 and LGR5 were unchanged (FIG. 1F, FIG. 2I). Gene set enrichment analyses confirmed that the Notch pathway was selectively upregulated, PI3K pathway downregulated, and other unrelated pathways, e.g., Hedgehog, were unaffected (FIGS. 2J-2L). Within this 48 hour timeframe, HNF1B expression was upregulated 2.88-fold in PI3K inhibited samples, but HNF4A was not detected at day 12 (TPM<1.7; FIG. 1F, FIG. 2I). These data show that organoids upregulate JAG1 and HNF1B in response to transient PI3K inhibition.

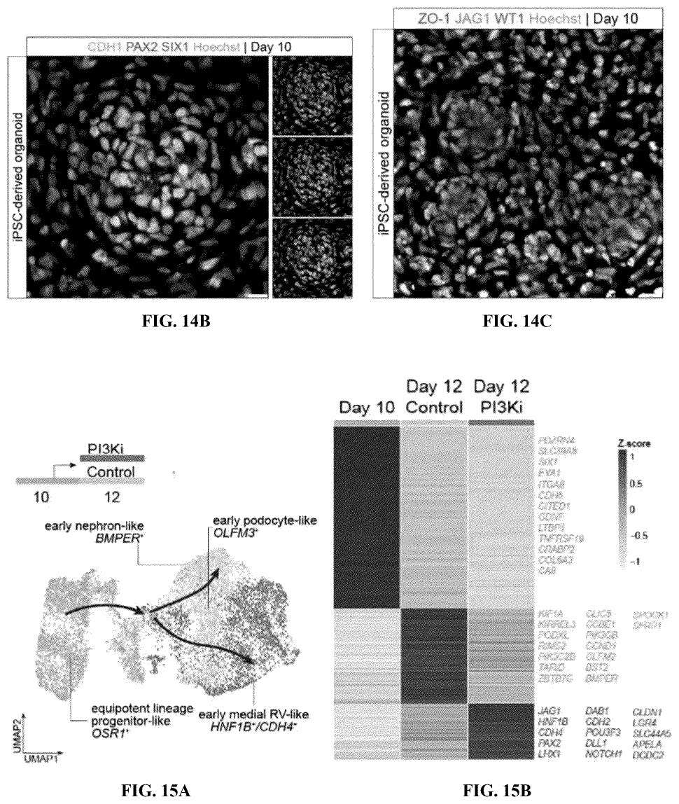

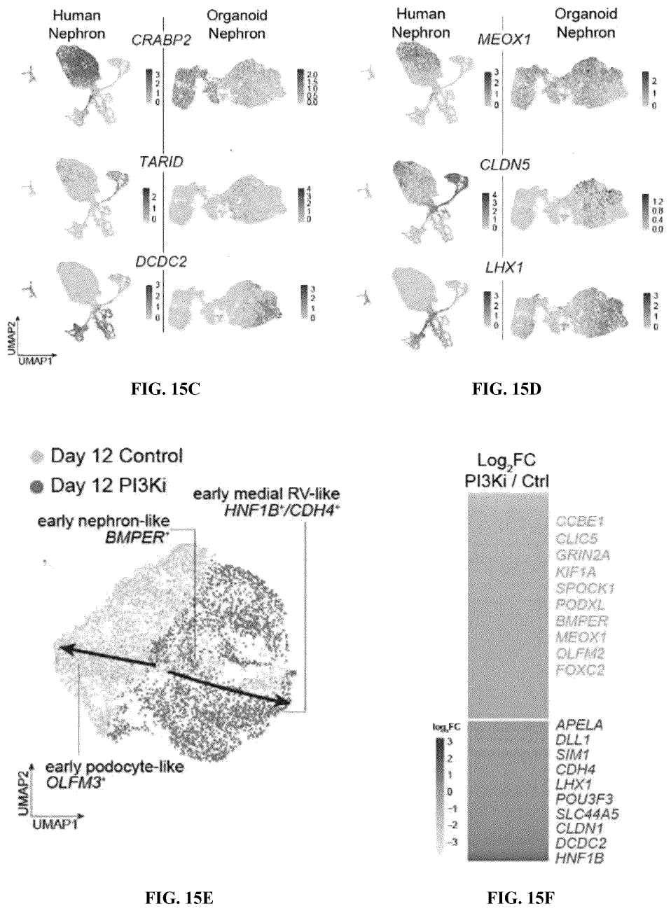

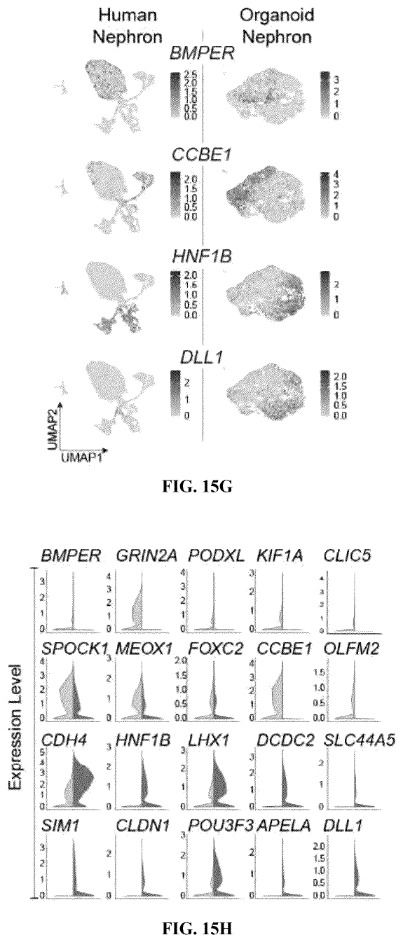

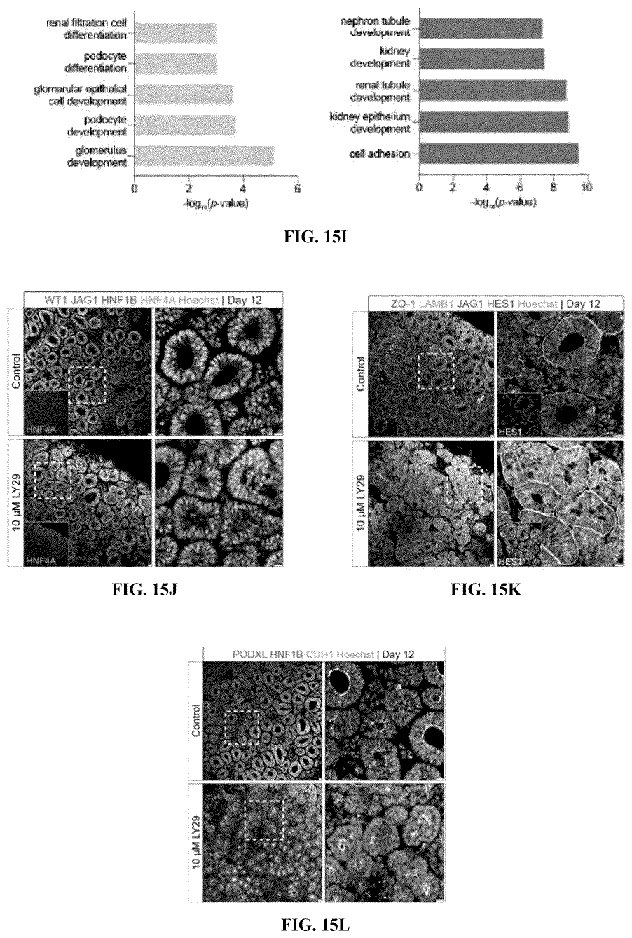

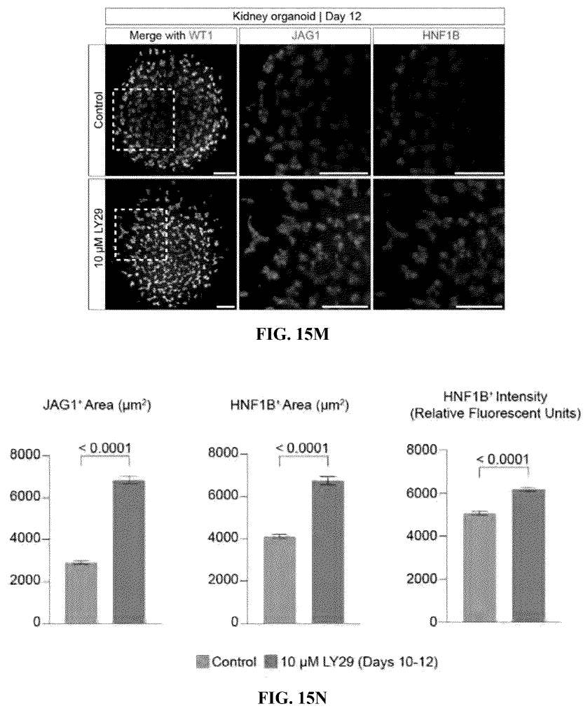

To unbiasedly investigate proximalization in organoid nephron cells, the study performed single-cell RNA-sequencing on day 10 untreated, day 12 control, and day 12 Ly29-treated organoids (FIG. 15A). Differential gene expression analyses revealed distinct transcriptional profiles (FIG. 15B). Day 10 organoid nephrons were enriched for nephron progenitor markers SIX1, CITED1, MEOX1, and CRABP2. By day 12, control organoid nephrons upregulated podocyte markers TARID, an antisense lncRNA that activates podocyte-specific TCF21 expression, as well as podocyte markers PODXL, CLDN5, KIRREL3, and CLIC5. Ly29-treated organoid nephrons however, showed increased expression of epithelialization and nephron tubule markers, including CDH4, DCDC2, JAG1, LHX1, and HNF1B, indicating a shift toward an epithelial nephron tubule fate (FIGS. 15B-15D).

To directly assess the impact of PI3K inhibition on differentiation, the study compared day 12 control and Ly29-treated organoid nephron cells (FIG. 15E). Differential gene expression analyses substantiated strong divergent profiles of day 12 control and Ly29-treated samples with control cells enriched for progenitor genes MEOX1 and BMPER, and podocyte markers PODXL and CCBE1, while Ly29-treated cells upregulated epithelial and nephron tubule markers, including CDH4, LHX1, and POU3F3; Notch pathway ligands JAG1 and DLL1; and the HNF4A-binding transcription factor HNF1B23 (FIGS. 15E-15G). Most HNF1B⁺ cells at day 12 originated from Ly29-treated nephrons, with similar patterns observed for other key markers (FIG. 15H). Unbiased gene ontology analyses of the top 50 most differentially expressed genes for each condition showed day 12 control cells being enriched for processes related to podocyte differentiation and glomerulus development, while day 12 Ly29-treated cells were enriched for nephron tubule development, kidney epithelium development, and cell adhesion (FIG. 15I). Together, these findings suggest that day 12 control cells are primed for podocyte development, while PI3K inhibitor treatment shifts cells toward an epithelial nephron tubule fate, with strong upregulation of Notch and pre-proximal tubule markers, reflecting patterns observed in human proximal nephron development.

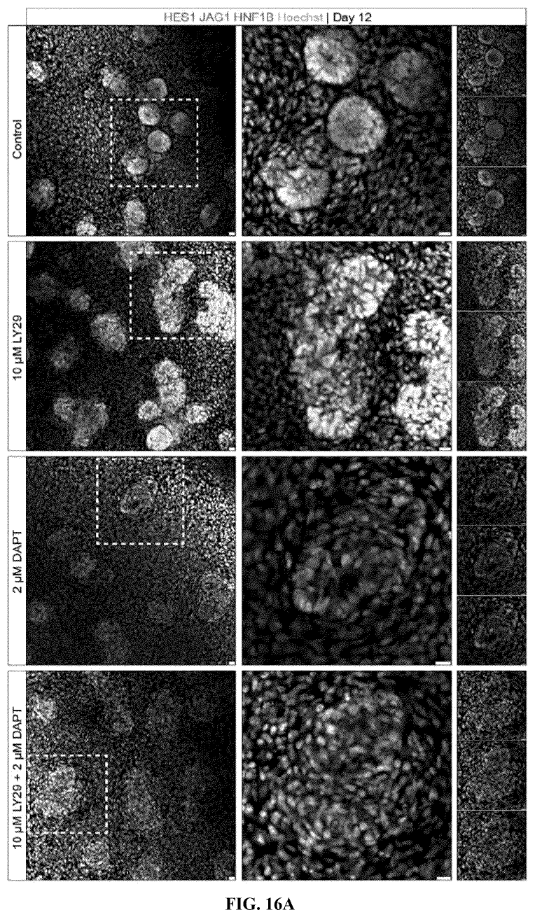

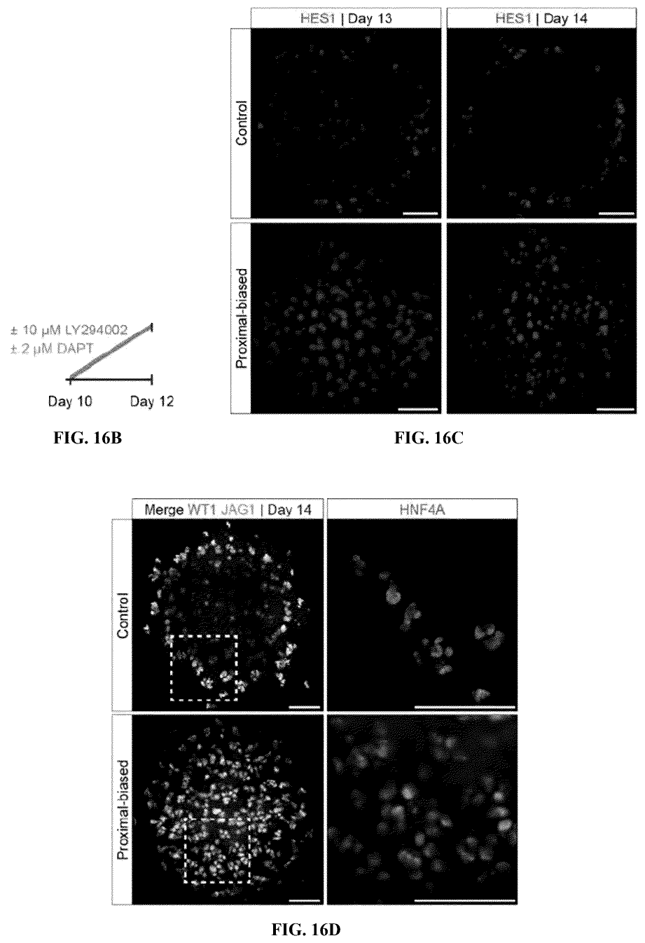

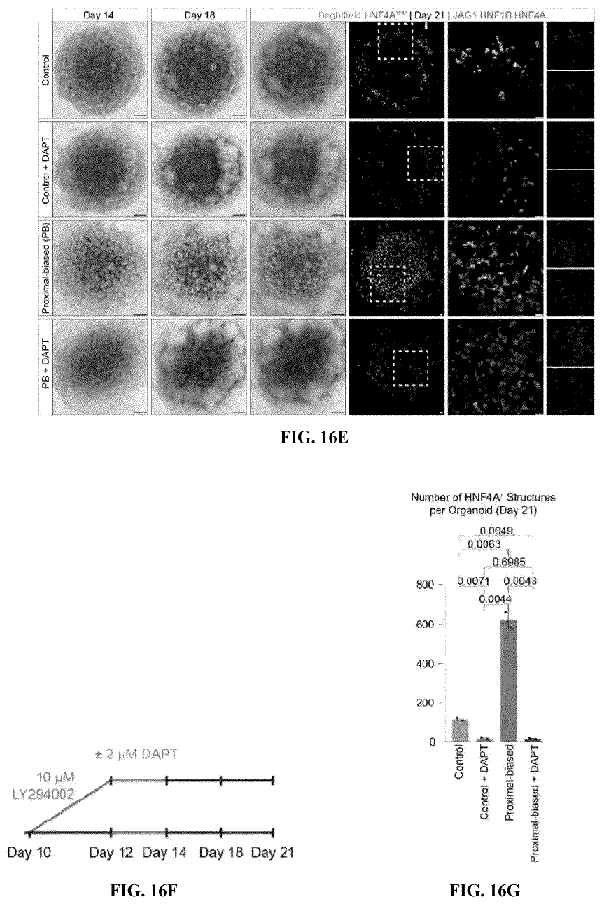

To validate and extend these findings, the study performed whole-mount immunofluorescence in organoids using two iPSC lines to confirm phenotype robustness. Consistent with single-cell RNA-sequencing results, day 12 Ly29-treated cells showed strong upregulation of JAG1 and HNF1B throughout organoid nephrons, while control organoids exhibited mosaic detection of HNF1B and lower levels of JAG1. HNF4A was not yet detected in either condition at day 12 (FIG. 15J). The upregulation of JAG1 was accompanied by increased detection of Notch target HES1, which remained low in controls (FIG. 15K). By day 12, a fully formed basement membrane, marked by LAMB1, was observed in both control and Ly29-treated organoids (FIG. 15K). Co-upregulation of epithelialization marker CDH1 and HNF1B was observed, with increased intensity in Ly29-treated samples (FIG. 15L). Quantitative assessment of JAG1 and HNF1B abundance in day 12 organoids showed increases in JAG1 and HNF1B in Ly29-treated organoids. Their upregulation occurred in nephrons alongside elongation (HNF1B 1.64-fold size increase, p-value <0.0001; JAG1, 2.36-fold size increase, p-value <0.0001), and the abundance of HNF1B also increased within each nephron (1.22-fold increase, p-value <0.0001) compared to controls (FIGS. 15M-15N). Consistent with PI3K inhibition resulting in Notch signaling linked to HNF1B expression, nephrons did not upregulate HNF1B or HES1 when cultured with Notch/gamma-secretase inhibitor DAPT, either with or without Ly29 (FIGS. 16A-16B). The increase in HES1 upon Ly29 treatment persisted through differentiation days 13 and 14; 1 and 2 days after the removal of the inhibitor. Transiently blocking PI3K signaling therefore drives Notch signaling and HES1 protein production throughout organoid nephrons, while control organoid nephrons exhibit low and non-uniform HES1 protein levels (FIG. 16C). Importantly, inhibition of PI3K signaling altered the structural dynamics of the organoids, leading to the emergence of JAG1⁺/HNF1B⁺ structures distributed across both the periphery and center of the organoid discs, while control organoids predominantly exhibited tubulogenesis biased towards the periphery (FIGS. 15J-15M, FIG. 16C).

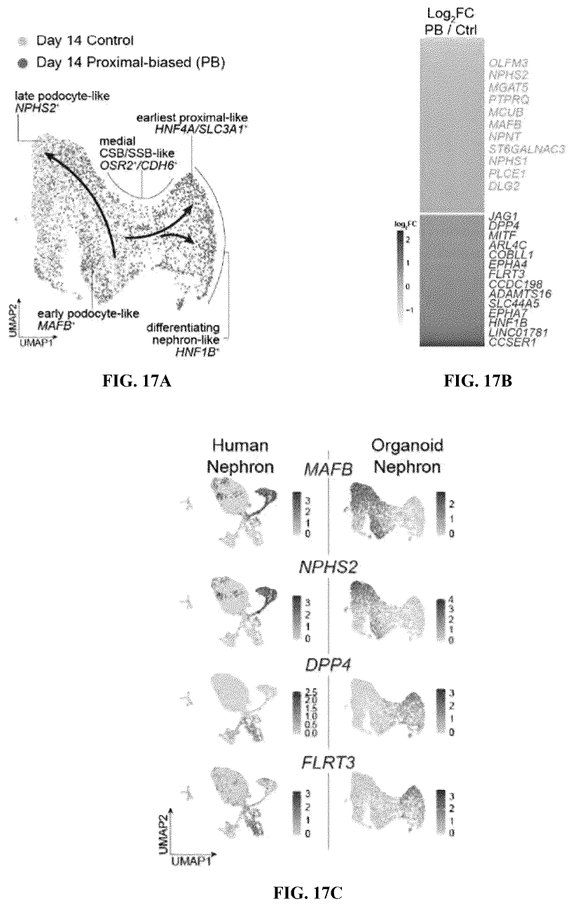

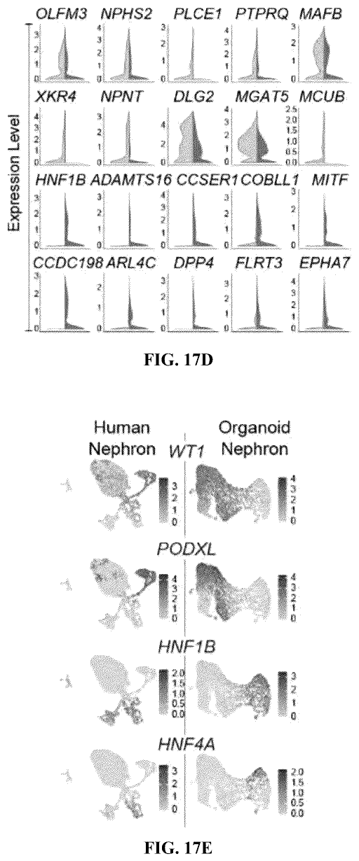

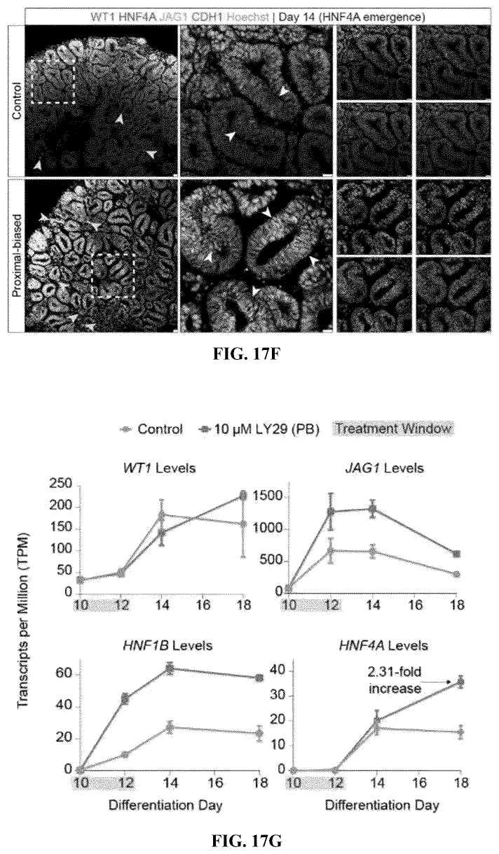

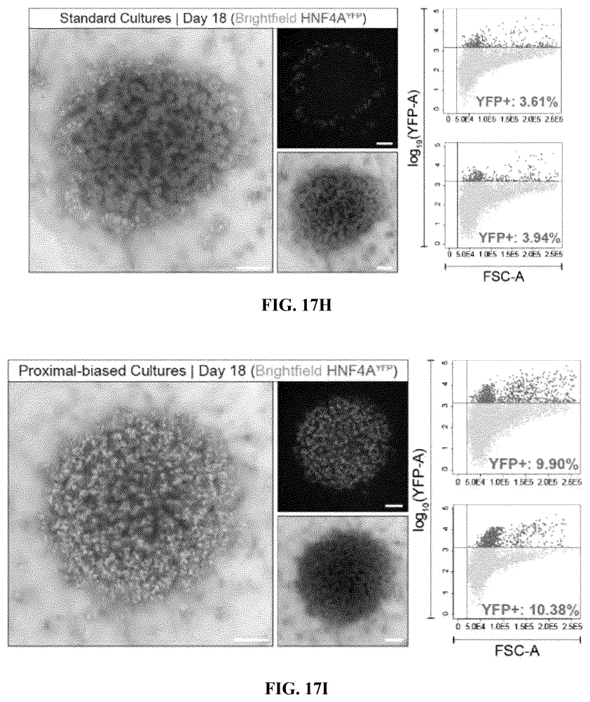

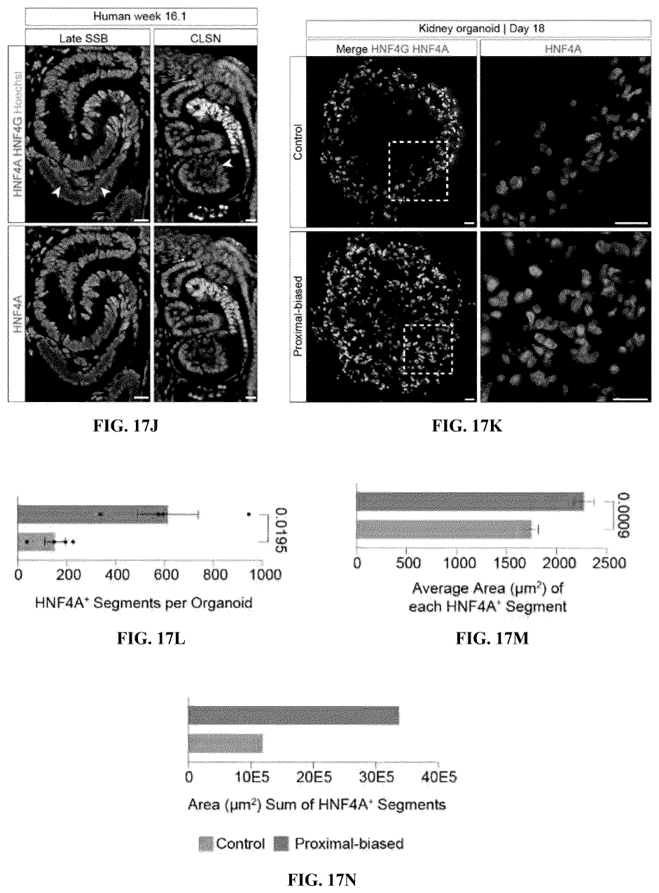

To scrutinize how cells differentiate from their day 12 states, the study performed single-cell RNA-sequencing at day 14, two days after PI3K inhibitor removal (FIG. 17A). Differential gene expression analyses revealed that control organoid nephron cells largely follow a podocyte differentiation program, marked by enrichment of podocyte-associated genes MAFB, PTPRQ, NPHS2, PODXL, and WT1 (FIGS. 17B-17C). In contrast, day 14 Ly29-treated organoid nephron cells exhibited proximal-biased (PB) differentiation with increased expression of human medial SSB and proximal nephron genes, such as brush-border markers DPP4 and FLRT3, along with sustained expression of HNF1B and JAG1 (FIGS. 17B-17D). The distinct expression patterns of these markers between control and treated cells suggest each condition maintains its differentiation program. Notably, HNF4A—previously undetected at day 12—was now observed in both conditions but prominent in day 14 Ly29-treated cells (FIG. 17E). The study confirmed the proximal-driving effect of PI3K inhibition using a second inhibitor of PI3K signaling that differs structurally from Ly29, GDC-094136. Overall, these results suggest that early PI3K inhibition promotes a population of progenitor-like cells primed for HNF4A⁺ proximal nephron emergence.

The transition from a JAG1⁺/HNF1B⁺ state to HNF4A⁺ proximal cells, as was observed in vivo (FIG. 1B), is mirrored in organoids. Two different human iPSC lines confirmed onset of HNF4A expression by day 14, detected as compact, nascent structures not yet elongated into tubules (FIG. 16D, FIG. 17F). In control organoids, HNF4A expression was observed primarily at the periphery. In contrast, PI3K inhibitor-treated organoids exhibited extensive HNF4A upregulation throughout the organoid (FIG. 16D, FIG. 17F), indicating a broader activation of proximal fate. Bulk RNA-sequencing performed over the differentiation timeline shows that by day 18, six days after PI3K inhibitor removal, HNF4A transcript levels were increased 2.31-fold (p-value: 0.0318) in PB organoids compared to controls, indicating that the early upregulation in HNF1B/HNF1B and JAG1/JAG1 seen at days 12 and 14 transition into a sustained HNF4A⁺ proximal fate (FIG. 17G). To validate these findings, the study used HNF4A-YFP reporter iPSCs to label proximal tubule cells with yellow fluorescent protein (YFP). FACS analyses at day 18 revealed a 2.69-fold increased abundance (p-value: 0.0021) of YFP⁺ cells in treated organoids compared to controls. Among the total dissociated organoid cells, 10.14% of PB organoids were YFP⁺, compared to 3.78% in controls. Consistent with day 14 observations, YFP⁺ proximal nephron formation remained localized to the periphery in control organoids, while HNF4A-YFP was upregulated throughout the entire organoid in day 18 PB samples (FIGS. 17H-17I).

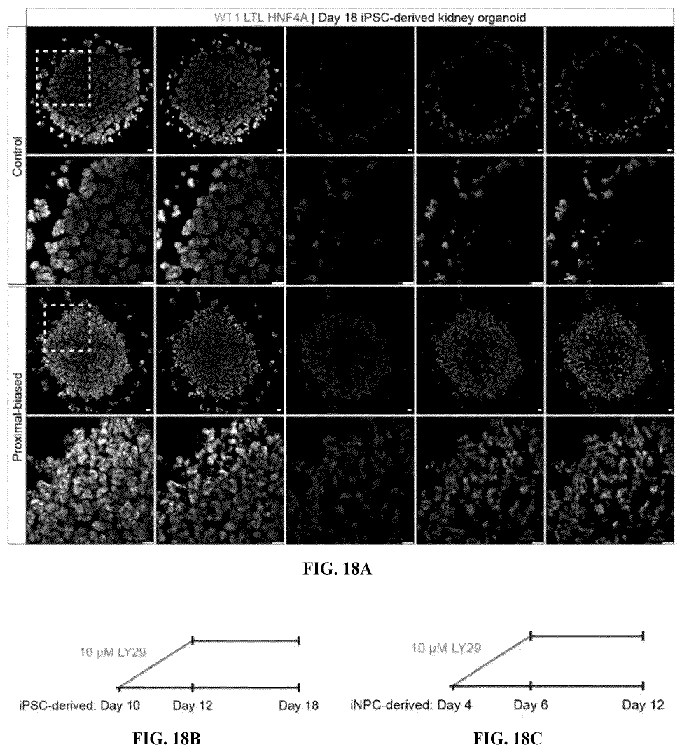

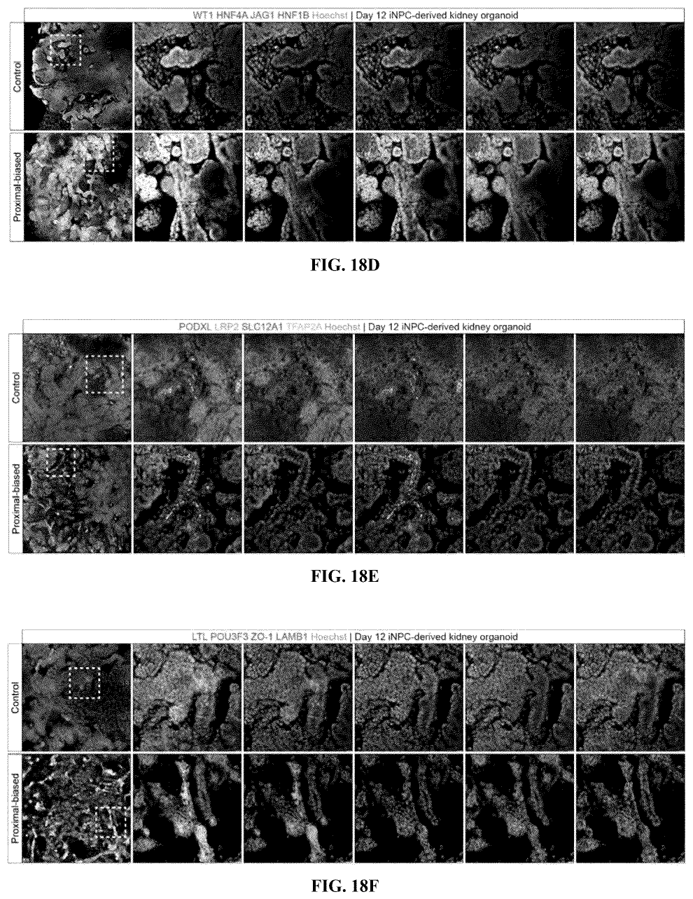

In vivo, the paralog of HNF4A, HNF4G, is co-upregulated when the proximal precursor domain elongates (FIG. 17J). As PB organoid nephrons differentiated, they upregulated HNF4A/HNF4A and HNF4G/HNF4G (1.72-fold increase in transcript levels compared to controls, p-value: 0.0305) (FIG. 17G, FIG. 17K), while displaying unaltered WT1 RNA and protein levels (marking podocytes) (FIGS. 17F-17G, FIG. 16D). PB organoids increased the total number of HNF4A⁺ segments per organoid (4.02-fold increase, p-value: 0.0195), the average size of individual HNF4A⁺ segments (1.30-fold increase, p-value: 0.0009), and therefore the sum area of HNF4A⁺ organoid nephron segments (2.83-fold increase for n=3 organoids) (FIGS. 17L-17N). To verify that HNF4A⁺ segments in control and PB organoids correspond to proximal tubules, the study co-stained for HNF4A and Lotus tetragonolobus lectin (LTL), a broadly used proximal tubule marker, observing codetection of these markers in both conditions (FIG. 18A). HNF4A⁺ tubules were labelled with LTL, however LTL is a less-preferred marker for human proximal tubules given broad non-specific labelling of other nephron segments.

To determine whether HNF4A⁺ cell differentiation is Notch-signaling dependent, the study treated HNF4A-YFP control and PB organoids with DAPT for 48 hours between days 12 and 14, after Ly29 withdrawal (FIGS. 16E-16F). Inhibiting Notch signaling after PI3K inhibitor treatment reduced HNF4A-YFP in both control and PB conditions. PB organoids formed YFP⁺ structures throughout, while control organoids displayed YFP signal primarily at the periphery. In DAPT conditions, YFP− structures still developed but were HNF1B⁺/JAG1⁺/HNF4A− (FIG. 16E), indicating Notch signaling is required for the transition from HNF1B⁺ to HNF4A⁺ cells, as anticipated. PB organoids contained more HNF4A⁺ structures than other conditions, and controls formed more HNF4A⁺ structures than DAPT-treated conditions (pvalues <0.01) (FIG. 16G). These data confirm that Notch is required for HNF4A⁺ proximal cell formation and suggest that PI3K inhibition promotes proximal cell fate through Notch-dependent priming of HNF1B⁺/JAG1⁺ cells.

To further test the robustness of proximal biasing and its targeting of nephron cells, the study applied the approach to the recently developed induced human nephron progenitor cell (iNPC) propagation system. iNPCs were differentiated from H1 embryonic stem cells, expanded, aggregated, and differentiated into nephrons. The resulting organoids are derived from SIX2⁺ iNPCs and highly enriched for nephron structures. To stage-match iNPC and kidney organoid protocols, iNPC organoids were treated for 48 hours with Ly29 between differentiation days 4-6, at which point the organoids show a gene signature characteristic of the pretubular aggregate and renal vesicle-stage nephron (PAX8⁺/WNT4⁺/CRYM⁺), equivalent to day 10 iPSC-derived kidney organoids (FIGS. 18B-18C). Six days after the removal of the PI3K inhibitor, day 12 iNPC derived organoids showed more abundant proximal HNF1BHIGH/HNF4A⁺/LRP2⁺/LTL⁺ segments compared to controls (FIGS. 18D-18F). PB iNPC-derived organoids formed PODXL*/WT1⁺ podocyte-like structures and tubules displayed apical-basal polarities marked by ZO-1 and LAMB1 (FIGS. 18D-18F). Minimal distal TFAP2A⁺ or loop of Henle-like SLC12A1⁺ structures were observed in either condition (FIGS. 18D-18F). Collectively, these data show that the proximal biasing approach is applicable to other kidney systems and confirms that the mechanism acts directly on nephron cells rather than other kidney cell populations.

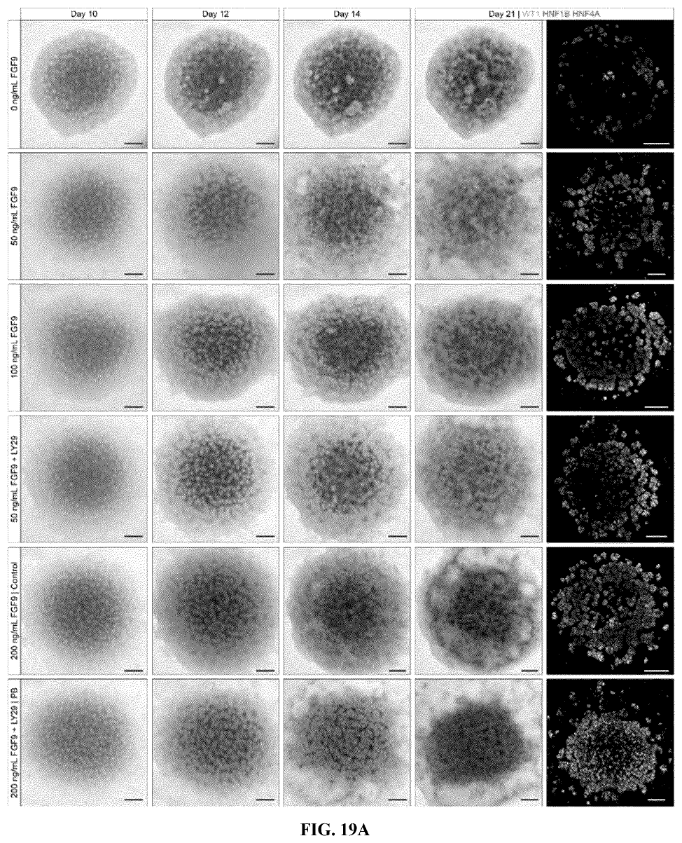

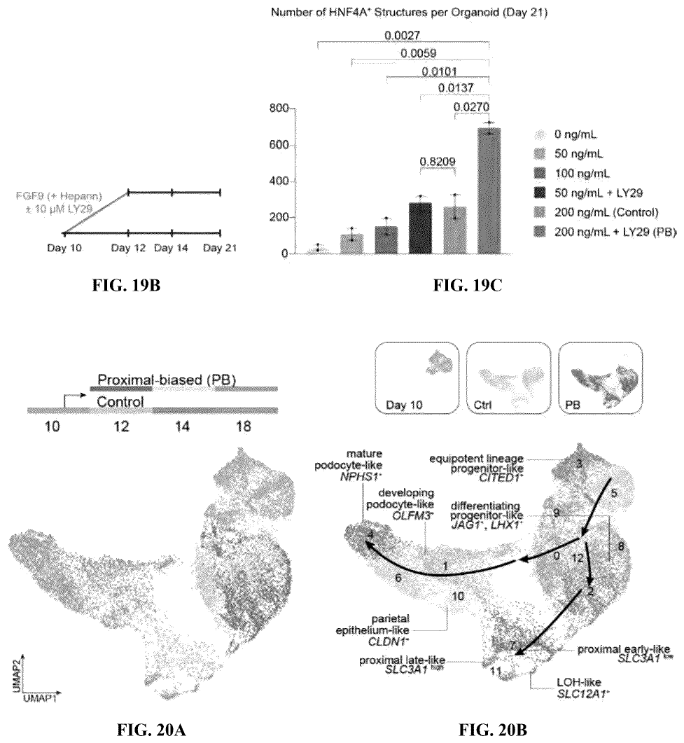

Given that PI3K signal transduction can act downstream of FGF signaling, and that FGF9 is a component of the organoid differentiation protocol, the study tested whether decreasing FGF9 concentrations during organoid development can mimic the effect of PI3K inhibition. The study explored reducing FGF9 concentrations from days 10 to 12 (FIGS. 19A-19B) by decreasing concentrations from standard 200 ng/mL FGF94,27 to concentrations between 0 to 200 ng/mL. Complete removal of FGF9 severely impaired nephron differentiation, and few WT1⁺, HNF1B⁺, or HNF4A⁺ nephrons formed. Increasing FGF9 concentrations from 0 ng/mL to 50 ng/mL or 100 ng/mL trend-improved development of nephrons above the 0 ng/mL state, but they nevertheless remained below standard (FIG. 19C). This suggests that lowering FGF9 concentrations does not mimic the effects of PI3K inhibition. To further test this, the study simultaneously inhibited PI3K in organoids exposed to only 50 ng/mL FGF9. In these conditions, nephrogenesis was rescued to control organoid levels (p-value: 0.8209), but only in the PB condition (200 ng/mL FGF9⁺ 10 μM Ly29) was there a significant increase in HNF4A⁺ structures above standard control conditions (FIG. 19C, p-value: 0.0270). Collectively, these findings demonstrate the importance of FGF9 for normal differentiation and decouple the proximal-biasing mechanism of PI3K inhibition and FGF9 signaling.

Enriching a functional proximal precursor identity in kidney organoids: To examine the differentiation outcomes of PB cells, the study captured and integrated single-cell RNA-sequencing data from control and PB organoids across days 10, 12, 14, and through to 18 (FIGS. 6A-6B). The data from nephrogenic cells highlight the divergent day 12 signatures with distinct transcriptional states exhibited by controls (primed towards TARID⁺/OLFM3⁺ podocytes) and Ly29-treated nephrons biased to generate nephron tubule-forming cells (HNF1B⁺/LHX1⁺/DLL1⁺) (FIG. 6C, FIGS. 20A-20B). Inferred developmental trajectories from progenitors through to HNF4A⁺ cells suggests day 10 cells transition from progenitors and post-proximalization, gradually differentiate into proximal nephron lineages by day 18, as evident from a continuum of transcriptional states (FIGS. 6D-6F). While organoid nephrogenic cells partially separated by sample timepoint, overlap was also observed, indicating that early and transient Ly29 treatment coaxed cells towards a PB state, increased the number of these cells, but did not introduce sufficient transcriptional changes to otherwise separate clusters. Highlighting the strong biasing introduced by PI3K inhibition, 70.8% of all HNF4A⁺ proximal tubule precursors originated from the PB condition, with similar ratios for HNF1B and JAG1 (FIG. 20C).

To further understand the cellular composition of the control and PB organoid nephron cells, the study compared their transcriptional profiles with in vivo nephron cells (FIG. 2A, FIG. 6G). Organoid nephrogenic cells largely co-clustered with human nephron cells (FIGS. 6H-6I). Sample contributions for each cluster were quantified (FIG. 6I), showing organoid podocytes (clusters 2 and 3: MAFB⁺, OLFM3⁺, NPHS2⁺), early proximal (cluster 12: HNF1B⁺, JAG1HIGH), proximal (cluster 10: HNF1B⁺, HNF4A⁺, SLC3A1⁺), and loop of Henle precursor (cluster 9: SLC12A1⁺, MAL⁺) cells clustered with in vivo cells, while in vivo human CITED1⁺ nephron progenitor states (clusters 0 and 5) were poorly represented in day 10 organoids (cluster 1). This was likely since CITED1⁺ day 10 organoid cells also express PAX8 and LHX140, thus displaying differences to normal in vivo nephron progenitors (FIGS. 6G-6J). Distinct clusters were observed between day 12 control (clusters 7 and 8: PDGFRB⁺, PI3KHIGH), day 12 PB (cluster 4: JAG1⁺, DLL1⁺, NotchHIGH), and early human nephron (cluster 6: SNAI2⁺, DAPL1⁺) cells, pointing to transcriptional differences induced by Ly29 treatment (FIGS. 2J-2K, FIG. 6H). Distal human nephron cells (clusters 20, 19, and 16) separated from organoid nephron cells. Collectively, these data indicate that the PB organoids primarily generate in vitro equivalents of podocyte and proximal cells.

Validating these data in individual organoid nephrons showed consistent views (FIGS. 20D-20G). Morphologically, PB organoids display expansion of the HNF1B⁺/HNF4A⁺ identity throughout the tubular portion of the organoid nephron. Initially, PB organoids display a subtle increase in the WT1⁺ domain immediately following PI3K inhibition at day 12 (1.08-fold larger, p-value: 0.0015) (FIGS. 6K-6L). However, this effect is not sustained and by day 18 control nephrons display WT1⁺ renal corpuscle-like structures larger than those in the PB organoids (1.42-fold larger, p-value: 0.0002) (FIGS. 6K-6L, FIGS. 20D-20G). The persistent PB organoid nephron phenotype is an elongated HNF4A⁺ proximal domain (FIGS. 17K-17N, FIG. 20E, FIG. 20G) compared to controls (FIG. 20D, FIG. 20F). HNF4AYFP reporter organoids show YFP is gradually upregulated and increasing in abundance between days 15-18, expanding throughout PB organoids, while in control organoids, YFP⁺ is restricted to the periphery (FIG. 20H).

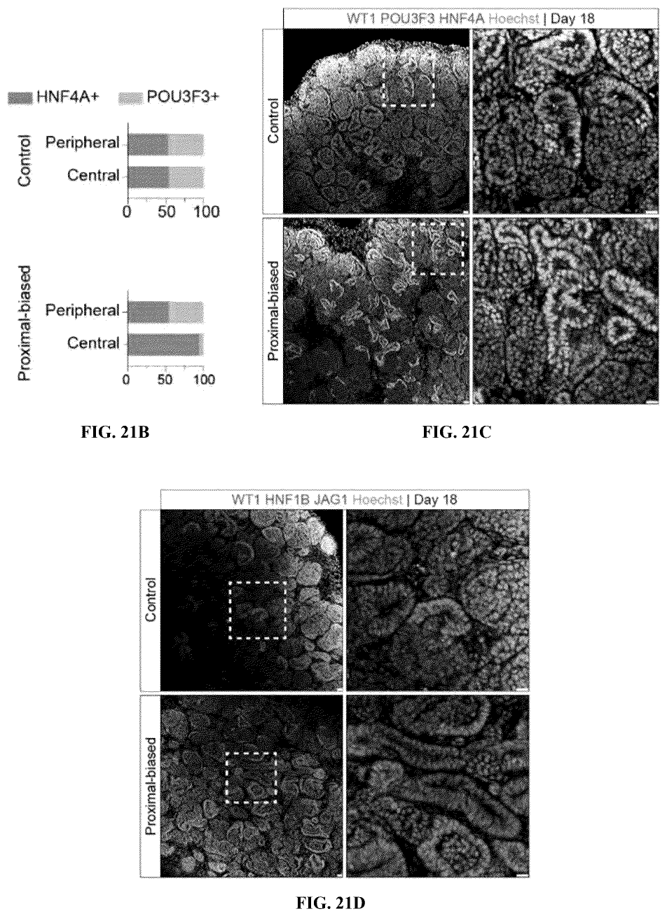

To broadly assess nephron differentiation outcomes in PB organoids, the study immunostained for proximal and distal segment-specific markers. At day 14, proximal tubule maker LRP2/Megalin (proposed HNF4A target16) was weakly detected. By day 21 LRP2/Megalin⁺ structures were abundant throughout PB organoids but not in controls (FIG. 20I). WT1⁺ structures adjacent to LRP2/Megalin⁺ tubules were smaller in PB organoids, consistent with earlier observations (FIGS. 6K-6L). TFAP2A⁺ distal-like structures were sparse across both differentiation conditions, with no significant difference between control and PB organoids (Day 14: no change±0.149 SEM; Day 21: no change±0.197 SEM) (FIG. 20I, FIG. 20K). The spreading of nephron-forming events from periphery to center of each organoid upregulated HNF4A centrally (FIGS. 21A-21B). Nephrons at the organoid periphery increased expression of transcription factors HNF4A and POU3F3 (FIGS. 21A-21B), the latter detected in segment 3 (S3) in the in vivo proximal tubule28, and otherwise distally. The upregulation of POU3F3 was confirmed by bulk RNA-sequencing. At day 21, SLC12A1⁺ loop-of-Henle-like structures were detected in both conditions, with a slight increase in abundance in PB organoids (1.63-fold higher, ±0.464 SEM). Proximal LRP2/Megalin⁺ and HNF4A⁺ structures were more abundant (LRP2/Megalin: 3.06-fold higher 0.189 t SEM; HNF4A: 2.72-fold higher±0.299 SEM) and elongated in PB organoids compared to controls (FIGS. 20J-20K). HNF1B⁺ and HNF4A⁺ tubules elongated (HNF1B⁺: 2.44-fold longer, p-value: 0.0002; HNF4A⁺: 1.95-fold longer, p-value: 0.0013), while POU3F3⁺ tubules did not (p-value: 0.6069), supporting a role for HNF1BHIGH proximal conditions in promoting HNF4A⁺ tubule formation and elongation (FIG. 20L, FIGS. 21C-21D).

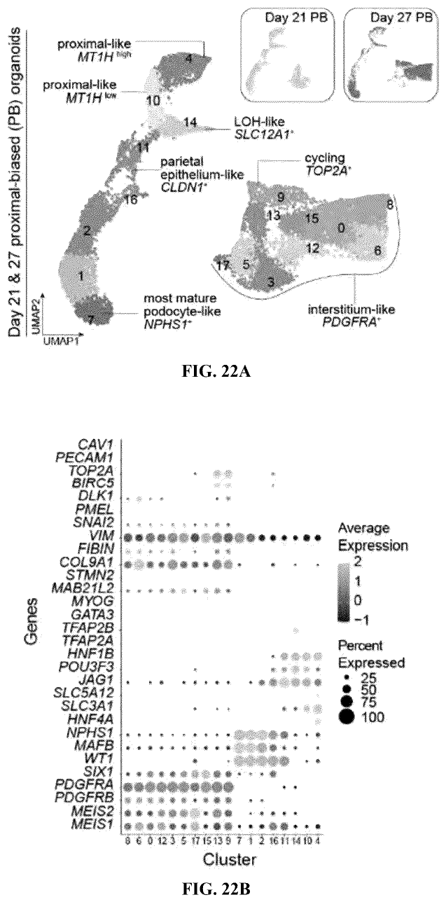

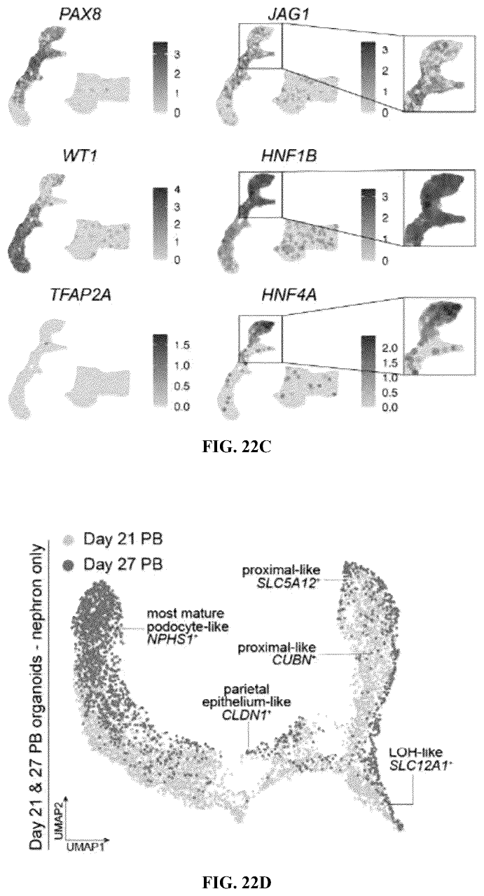

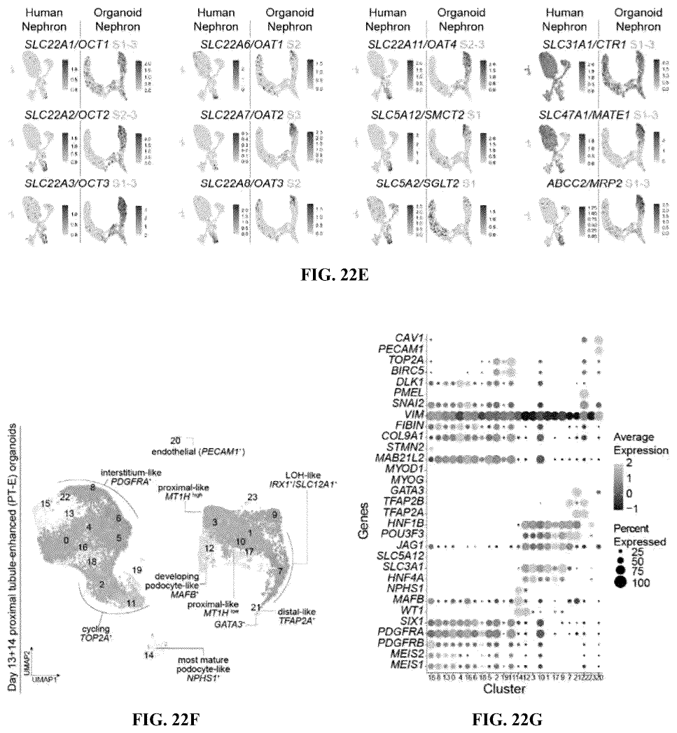

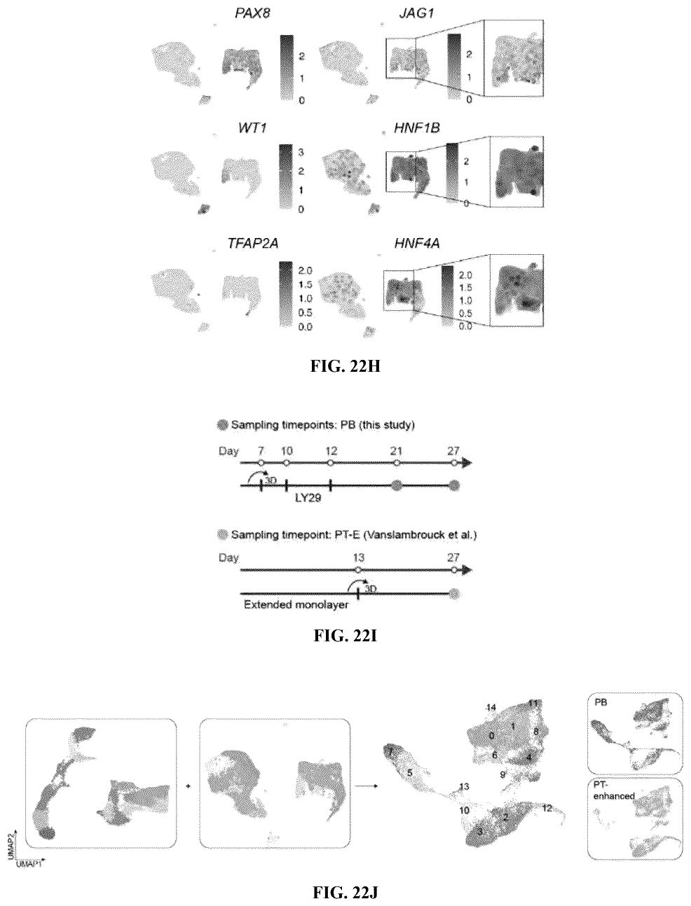

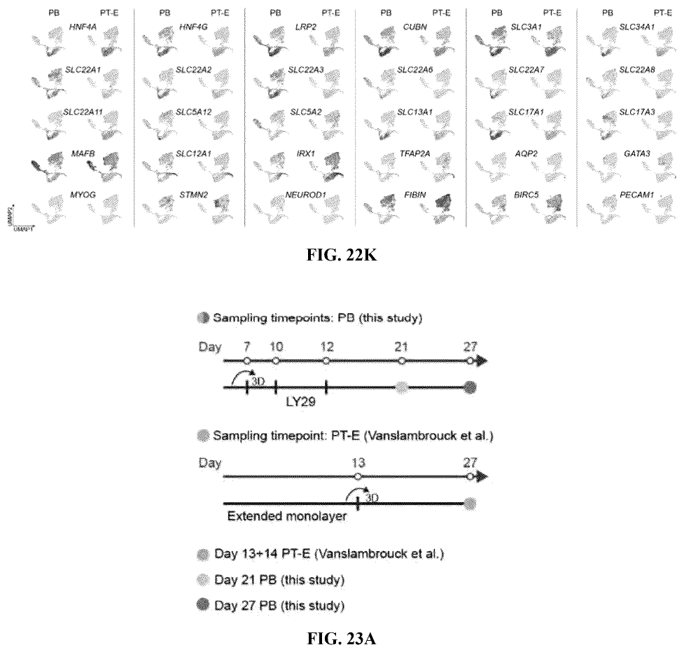

To assess maturation of the proximal tubule cells in the PB model, the study performed single-nucleus RNA-sequencing at days 21 and 27, marking 7 and 13 days after HNF4A first emerges. Integration of day 21 and day 27 PB organoid cells revealed overlap between time points, with cells belonging to either the nephrogenic or interstitial lineage, consistent with the model lacking off-target cell populations and being enriched for only metanephric mesenchyme lineages (FIGS. 22A-22C). Scrutiny of the nephrogenic lineage (nephrogenic subset of day 21 and day 27 PB organoids) showed co-clustering (FIG. 22D) and expression of genes involved in organic ion, glucose, and drug transport, suggesting PB organoid nephrons mature and express key proximal nephron solute carriers. Five out of six (83.3%) organic cation transporters showed expression in HNF4A⁺ cell clusters of day 21 and day 27 PB organoid nephrons, including SLC22A3/OCT3 and SLC22A2/OCT2 (FIG. 22E), the latter a known Hnf4a target. Similarly, five out of eight (62.5%) organic anion transporters displayed regional expression within HNF4A⁺ clusters of day 21 and day 27 PB organoid nephrons. Of these, SLC22A6/OAT1 and SLC22A8/OAT3 are Hnf4a/Hnf4a-dependent in the mouse16 and enriched in the maturing proximal nephron in the human (FIG. 22E). The glucose and monocarboxylate cotransporter SLC5A12/SMCT2, expressed in the maturing in vivo proximal nephron, was detected, yet glucose cotransporter SLC5A2/SGLT2 only sparsely so (FIG. 22E). Comparison with the developing human nephron indicated that PB organoids recapitulate key solute transporter expression patterns. These transporters emerged within HNF4A⁺ cell clusters, consistent with their known co-detection with Hnf4a/HNF4A in vivo, showing strong similarity between the PB organoid transcriptional profile and the in vivo proximal nephron (FIG. 22E).

To directly assess PB organoids against a leading model of proximal tubule cells, the study compared PB transcriptional profiles with data from the proximal tubule-enhanced (PT-E) kidney organoid protocol (FIGS. 22F-22H). Single-nucleus RNA-sequencing at day 21 stage-matched the PT-E organoids by their time in 3D culture (14 days post-initiation), and day 27 data stage-matched by total differentiation time (27 days, equivalent to the combined 13⁺14 days PT-E protocol; FIG. 22I). Both organoid types contained SLC12A1⁺ loop of Henle-like cells and WT1⁺ podocyte-like cells (FIGS. 22A-22C, FIGS. 22F-22H, FIG. 22K), but distal nephron markers TFAP2A, AQP2, and GATA3 were strongly detected in PT-E organoids, forming distinct groupings that were absent in PB organoids (FIG. 22K). Neither PB nor PT-E organoids displayed a pronounced muscle differentiation signature (MYOG⁺) (FIG. 22B, FIG. 22G, FIG. 22K).

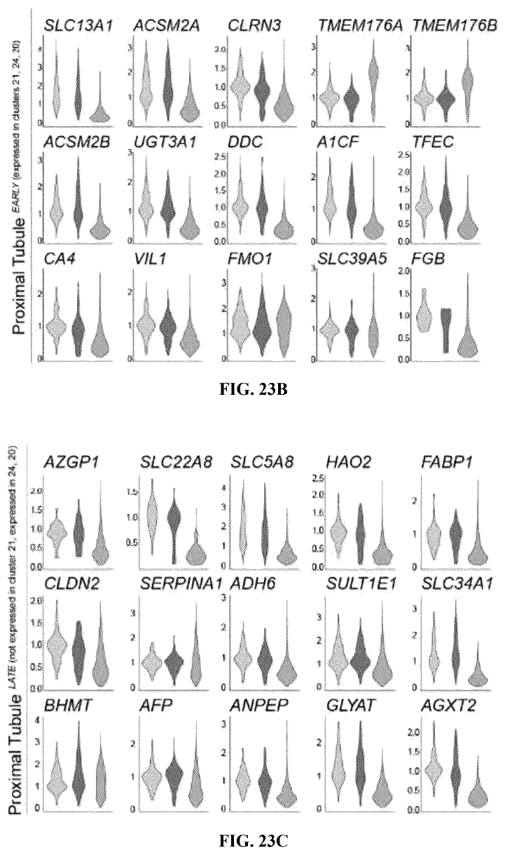

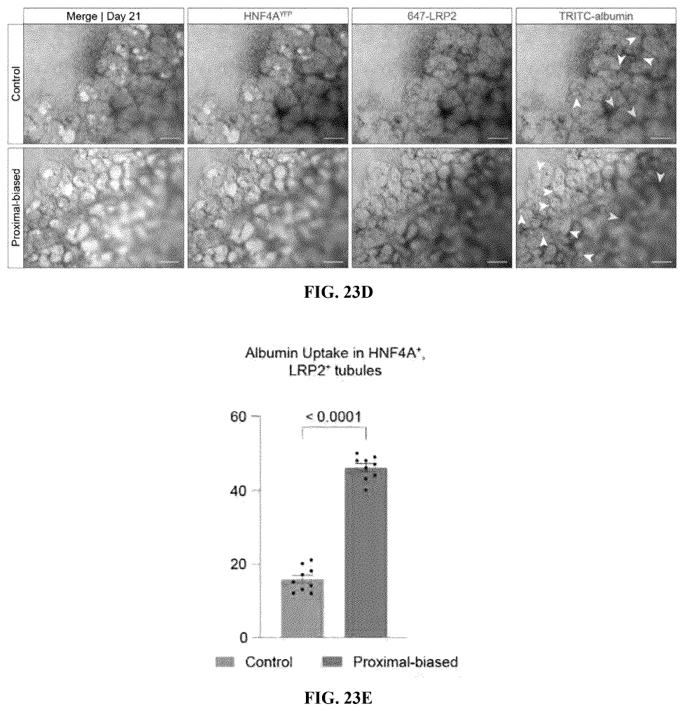

Extensive co-clustering was observed upon merging day 13⁺14 PT-E organoid cells with day 21 and day 27 PB organoid cells (FIGS. 22I-22J). To assess fidelity to normal development, the study leveraged HNF1B- and HNF4A-correlated genes to delineate ‘early’ and ‘late’ proximal nephron programs (early: genes detected in human clusters 21, 24, and 20; late: genes absent in human cluster 21 but detected in clusters 24 and 20) (FIG. 3A, FIGS. 23A-23C). All early and late proximal genes were present in both models, though detection frequencies varied. Early genes such as zinc transporter SLC39A5/ZIP5, brush-border marker VIL1, and monooxygenase factor FMO1 were frequently detected in both models, while genes such as sodium-sulfate cotransporter SLC13A1/NAS1, Hnf4a-dependent transcription factor TFEC, and deamination factor A1CF were more frequently detected in PB organoid cells (FIGS. 23A-23B). Late proximal tubule genes followed a similar trend. PB organoids exhibited higher detection frequencies of key late markers, including organic anion transporter SLC22A8/OAT3 and iodide transporter SLC5A8/AIT (FIG. 23C), while BHMT and AFP were detected at similar levels across cells from both organoid origins. Notably, these genes were detected in day 21 PB cells and remained detectable at day 27, indicating that transcriptional states emerge by day 21 and are maintained through to day 27 in the PB model.

Collectively, these data demonstrate that PB organoids establish broad proximal tubule transcriptional programs by differentiation day 21, which are maintained through day 27, while displaying minimal off-target and non-proximal populations.

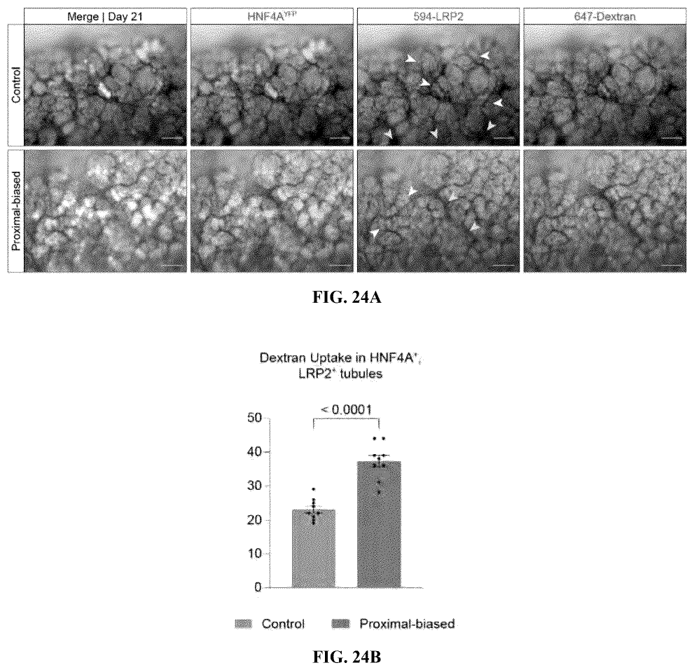

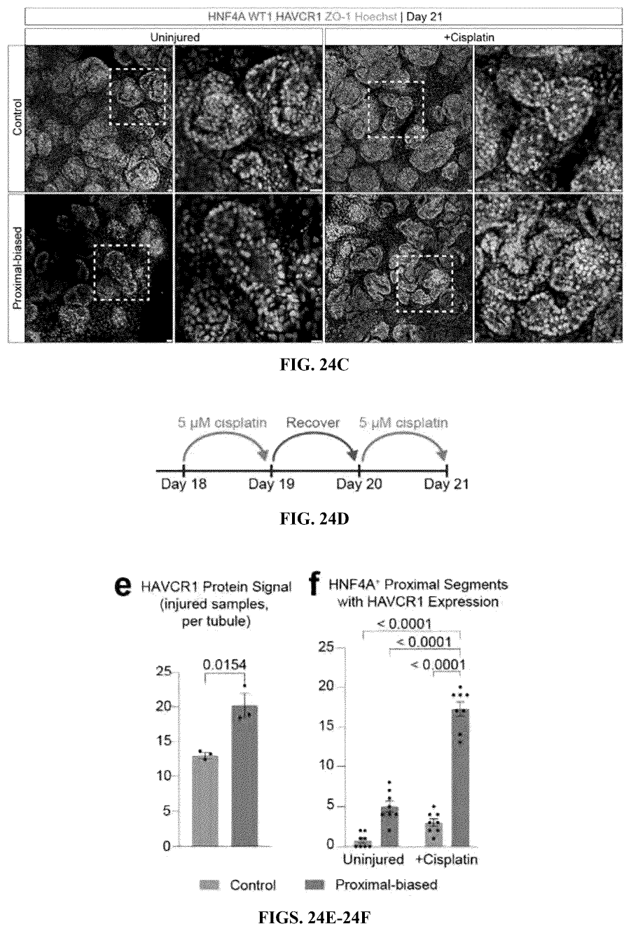

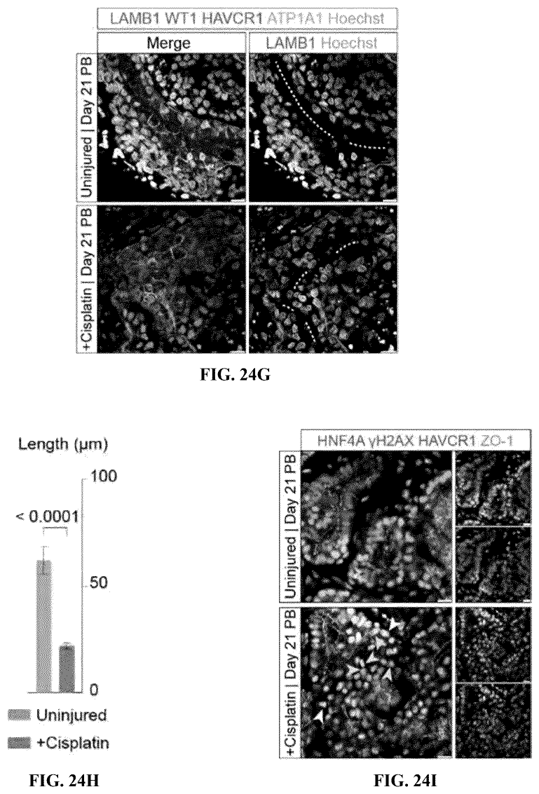

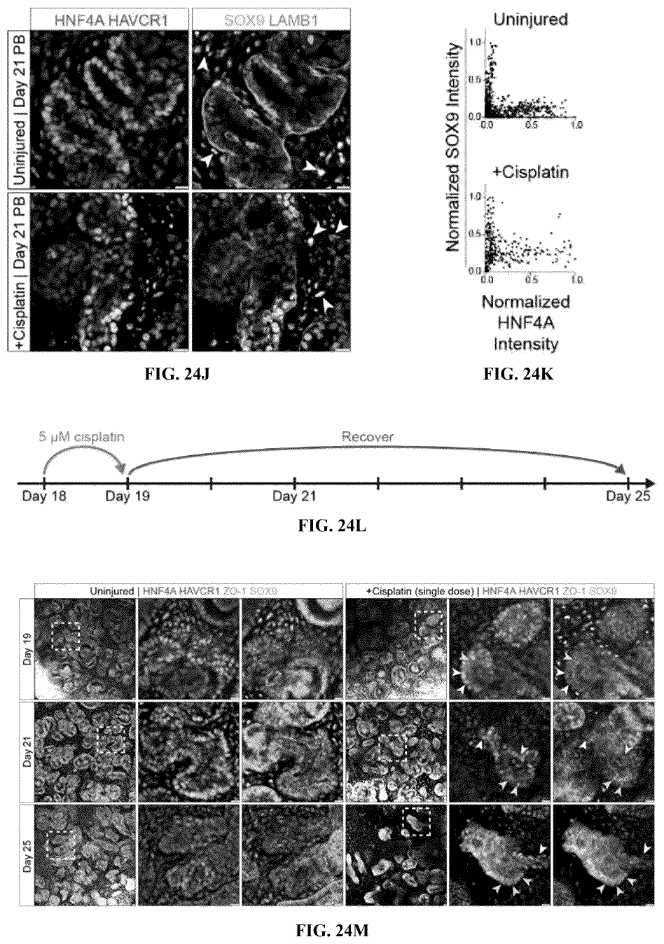

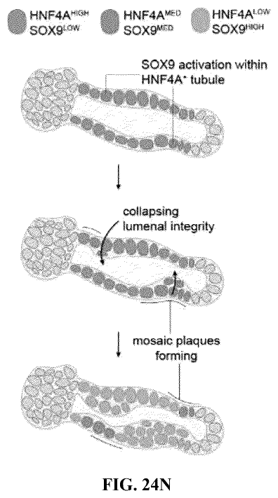

Modeling proximal tubule physiologies and cisplatin-induced injury in proximal-biased nephrons: Kidney organoid physiologies are often assessed by cells' ability to absorb and accumulate compounds selectively transported by solute carriers. Uptake of dextran and albumin are controlled by HNF4A-target genes LRP2/Megalin and CUBN16,42-45. Given that PB nephrons display increased and sustained expression of LRP2 and CUBN compared to controls (LRP2: 1.32-fold higher TPM; CUBN: 1.48-fold higher TPM, single-nucleus RNA-sequencing at day 21 and day 27, FIG. 22D, FIG. 22K), this raised the possibility of amplified proximal tubule function. To scrutinize this, the study performed live dextran and albumin uptake assays using HNF4AYFP reporter organoids co-cultured with fluorescently tagged LRP2/Megalin antibodies to label proximal tubules and apical surfaces. Day 21 PB organoids showed increased uptake of fluorescently tagged dextran and albumin in LRP2⁺/HNF4A-YFP⁺ tubules (FIGS. 24A-24B, 1.61-fold increase, p-value <0.0001; FIGS. 23D-23E, 2.92-fold increase, p-value <0.0001) compared to controls. Albumin and dextran were only detected within HNF4A-YFP⁺, LRP2/Megalin⁺ regions of nephrons with strong apical antibody labeling, indicating apical accessibility in live organoids and highlighting functional uptake capacity in proximal organoid nephrons (FIG. 23D, FIG. 24A).

The single-nucleus RNA-sequencing data from day 21 and 27 also revealed that PB cells express physiology-imparting genes, including those responsible for transporting cisplatin (FIG. 22E), a chemotherapeutic with severe nephrotoxic side-effects in the proximal convoluted tubule. Recent studies show that organoids are sensitive to cisplatin and respond by activating expression of early proximal tubule injury marker KIM1/HAVCR1 and display DNA damage akin to that observed in patients. Given that the PB model expresses organic ion and copper transporters OCT2/SLC22A2, OAT1/SLC22A6, OAT3/SLC22A8, CTR1/SLC31A1 (responsible for uptake) and MATE1/SLC47A1 and MRP2/ABCC2 (shuttling cisplatin to lumen) (FIG. 22E), the study tested whether PB cells model cisplatin-induced injury by treating PB and control organoids with two low staggered doses of cisplatin (5 μM) and assayed their response 3 days after initial injury (FIG. 24D). Cisplatin-injured PB organoids showed strong detection of KIM1/HAVCR1 in HNF4A⁺ cells compared to injured and uninjured control, and uninjured PB organoids (all p-values <0.0001) (FIG. 24C, FIG. 24F) and 1.56-fold higher protein abundance (p-value: 0.0154) (FIG. 24E). Cisplatin-injured nephrons also displayed loss of tubular integrity and apicobasal polarity as evidenced by depolarized distribution of apical and basal cell-surface markers ATP1A1 and LAMB1, respectively (FIG. 24G). Luminal integrity was lost in injured HAVCR1⁺ organoids, with collapsed apical-basal polarities and shorter lumen surfaces, while uninjured samples preserved tubular architecture and maintained continuous open lumens (FIG. 24H, 2.82-fold longer uninterrupted tubular lumens, p-value <0.0001).

Following injury, Hnf4a/HNF4A are mosaically downregulated in proximal tubules, and cisplatin-injured PB nephrons displayed HNF4A protein levels anticorrelating with DNA damage response marker γH2AX (r=−0.67, p-value <0.0001) (FIG. 24I). Interstitial γH2AX was also detected, highlighting broad DNA damage throughout organoids. In the mouse acute kidney injury (AKI) model, injured proximal tubular cells activate Sox968, and under conditions of ischemia-reperfusion injury and rhabdomyolysis-induced AKI, a persistent Sox9⁺ state is tightly linked to maladaptive repair, tubular degeneration, and fibrosis. PB kidney organoid nephrons activated SOX9 in response to cisplatin-induced injury with extensive codetection of HNF4A and SOX9, unlike controls (FIGS. 24J-24K). Reducing treatment to a single cisplatin-dose generated a mosaic response producing HNF4AHIGH/SOX9LOW cells 1 day post-injury, HNF4AMED/SOX9MED cells 3 days post-injury, and a broadly HNF4ALOW/SOX9HIGH state 6 days post-injury, indicating dynamic changes over time (FIGS. 24L-24N). Collectively, these data highlight the potential of the PB nephron proximal tubule cell model as a sensitive and rapid system to delineate nephrotoxic and acute kidney injury.

The PB organoid nephron therefore emerges as a robust method for generating in vivo-mimicking proximal tubule cells that replicate physiological processes and respond to injury-driving mechanisms.

### Discussion

This study developed an in vivo informed approach to mimic human proximal nephron precursor cells in organoids derived from multiple human iPSC lines by delineating early in vivo human proximal tubule development, comparing it to existing kidney organoid models, and thereafter driving changes in cell states to bias differentiation outcomes. This model generates nephron-like structures in thin self-organizing discs that preserve nephron 3D complexities, and takes advantage of the plasticity in nephron axial positional identities to drive organoid nephrons towards a proximal-biased (PB) precursor-like state where organoid cells sequentially activate transcription factors and function-imparting proximal tubule genes. This approach directly targets nephron cells, as iNPC-derived organoids—initially lacking interstitial or other non-nephron cell types—are sensitive to proximal-biasing. This system offers a reproducible model with improved fidelity to in vivo cells and expression of developmentally appropriate genes, including OCT and OAT family members, and a host of physiology-imparting solute carriers, making it a tool to study development, congenital disease, injury, and physiology.

This study demonstrates that in vivo, human proximal precursors develop through a series of cell-state transitions marked by activation of JAG1, followed by HNF1B, and production of HNF4A in the upper bend of the medial S-shaped body nephron. Genetic evidence from mice shows that Notch signaling is required for proximal nephron differentiation, nephron segmentation, and nephron number. Inhibition of PI3K resulted in a transient upregulation of Notch target genes and sustained upregulation of HNF1B. Inhibiting PI3K and simultaneously blocking Notch signaling in PB organoids prevented HNF1B expression, consistent with Notch1; Notch2 compound knockout mice failing to upregulate Hnf1b in nephrons. Blocking Notch at any point between early organoid nephron formation and HNF4A⁺ cell emergence was sufficient to prevent proximal-biasing and eventual emergence of HNF4A⁺ cells. In vivo, Hnf1b binds to and is required for expression of Hnf4a, with a conserved role for human HNF1B indicated by mutations in Hnf1b/HNF1B being linked to congenital kidney anomalies affecting tubulogenesis, as well as studies in kidney organoids. In this model, increased HNF1B/HNF1B expression and protein levels preceded detection of HNF4A/HNF4A, concurring with in vivo and in vitro studies showing HNF1B is essential for proximal nephron cell-fate development. Consistent with Notch signaling being required for the proximal nephron program, inhibition of Notch at day 12 reduced JAG1, HES1, and HNF1B protein levels in both control and PB settings. Of note, while the PI3K pathway can act downstream of FGF signaling (KEGG pathway: hsa04151), lowering FGF9 failed to replicate PI3K inhibition and instead broadly impaired nephron development and organoid cell growth, underscoring the known role of FGF9 as a growth factor for early kidney organoid nephrons.

While Hnf4a/HNF4A and Hnf4g/HNF4G are co-expressed in proximal tubules in vivo, evidence suggests a non-redundant relationship, with Hnf4a knockout mice and HNF4A knockout kidney organoids having prominent phenotypes. Loss of HNF4G however, only moderately alters kidney organoid development, and Hnf4g knockout mice display a phenotype in the intestine only when removed alongside Hnf4a, likely due to redundant roles of Hnf4 factors in enterocyte maturation. As part of the comparative analyzes between in vivo and in vitro, the study sought to understand how Hnf4a-dependent genes are expressed during development and in adult nephrons. It is interesting to note that a group of Hnf4a-dependent genes are not expressed in the adult, indicating temporally dynamic roles for Hnf4a, or that gene expression is mediated by indirect mechanisms requiring for example co-factors. Given the clear role of Hnf4a in injury and repair mechanisms, this is an area needing further scrutiny. Overall, these analyses point to that human orthologs of Hnf4a-dependent genes are usually expressed at much lower frequencies in organoids, which in turn suggests that most organoid systems at present only partially mirror the in vivo proximal program. However, the onset of HNF4A and HNF4G expression occurs within a short timeframe in the S-shaped body nephron and PB organoids do express both genes as the nephron tubules elongate, which mirrors the temporal in vivo sequence. Other kidney models generating HNF4A⁺ proximal cell-states have previously enriched kidney organoid cultures for the nephron progenitor state and subsequently optimized culture conditions to permit proximal-like cell development. In contrast, the PB system imposes a specific signaling event switching cells to a proximal developmental program, thus enabling reproducibility, synchronicity, and application across cell-lines, providing control over differentiation outcomes.

In all kidney organoid systems, it is unclear how JAG1 is initially activated in the forming nephron. In vivo, it is thought that Jag1 is bound by and sensitive to β-catenin mediated transcription and Jag1/JAG1 expression initiates in cells adjacent to the WNT9B secreting collecting duct. Recent spatial transcriptomic analyses of the pretubular aggregate indicates that JAG1 is activated in a small subset of cells closest to the collecting duct within a 1-2 cell-layer thick domain, suggesting a short-range collecting duct derived ligand is responsible for JAG1 expression. However, simultaneous inhibition of PI3K and activation of WNT/β-catenin results in strong and broad upregulation of Jag1 throughout the nephron progenitor population, indicating that Jag1 is controlled via multiple integrated pathways30. In the organoid setting, it remains unclear how the brief dosing with CHIR at day 7 (a GSK3-0 antagonist and WNT/β-catenin agonist) can control the emergence of JAG1⁺ nephron aggregates at day 10; a subject warranting further investigation. In favor of multiple pathways controlling JAG1 transcription during induction and nephron patterning, in vivo JAG1 expression follows an intriguing wave-like pattern where it is initially activated in collecting-duct adjacent pretubular aggregate cells and thereafter moves proximally through the nephron and is downregulated distally, suggesting multiple layers of control over this Notch ligand. Identifying the mechanisms that regulate JAG1 expression and activities will therefore be critical for understanding nephron induction and subsequent proximal-distal patterning.

In vivo, proximal tubule cells exhibit a well-defined apical-basal polarity, appropriately positioned solute carriers and transporters, vascularization, intraluminal solute flow, and sensitivity to nephrotoxic compounds. Kidney organoids display accessibility to apical surfaces, as evidenced by live culture of organoids with fluorescently conjugated LRP2 antibodies that bind to LRP2/Megalin at the apical proximal tubule surface. Here the study replicates this using HNF4A-YFP⁺ organoids and detect strong labelling of anti-LRP2-594 nm and anti-LRP-647 nm at the apical surfaces of HNF4A-YFP⁺ tubules, but not elsewhere, and co-labelling with fluorescently tagged albumin and dextran. While the mechanisms underpinning this apical accessibility in organoids remains unknown, antibodies, complex sugars, and proteins are all able to selectively label proximal tubule cells in kidney organoids and thus support their use in modeling nephron physiologies and drug toxicity.

Cisplatin-induced nephron injury has been demonstrated in organoids by several groups in systems with variable expression of cisplatin transporters and differing abundance of maturing proximal tubules. The PB model demonstrates expression of solute carriers capable of transporting cisplatin, including OCT2/SLC22A2, OAT1/SLC22A6, OAT3/SCL22A8, CTR1/SLC31A1, MATE1/SLC47A1, and MRP2/ABCC2 (FIG. 22E). PB nephrons are sensitive to cisplatin early in the differentiation protocol (day 18), at a low concentration (5 μM), in single doses, and in a short timeframe (72 hours), making the model suitable for screening experiments requiring short dosing plans and endpoints. While expression of HAVCR1/KIM1 was specific to HNF4A⁺ cells, DNA-damage marker γH2AX was detected throughout organoids, corroborating recent findings of potential broad cellular toxicity14. As injured PB nephrons upregulate SOX9, display apicobasal tubular collapse, and resolve into a persistent SOX9⁺ state 6 days post-injury, this resembles the recently identified Sox9ON-ON switch that marks maladaptive repair in mice. Closer scrutiny of the transcriptional state and dynamics of SOX9 activation and HNF4A downregulation, coupled with experiments critically assessing the regenerative potential of PB organoids will improve understanding of strengths and limitations of modeling cisplatin induced injury in organoid-derived proximal tubules. Further, while cisplatin transporters are now detected, it will be important in future experiments to determine how they function in this model.

The current PB model represents a cell-state that is best estimated to resemble a post-capillary loop stage nephron based on the expression of solute carriers during the day 21-27 period. A full complement of mature nephron markers, such as SGLT2/SLC5A2, remain elusive in kidney organoids, and the data suggest that generating a mature proximal tubule will require further modifications including tuning of culture conditions to drive maturation, elongation, and separation of proximally distinct segments. In comparison to other PT-enriching models, the PB models displays minimal development of non-kidney lineages such as cartilage, neuron, and muscle and addressing why these are generated in some protocols and not others remains an important future goal.

Beyond manipulation of pathways, the PB model currently lacks physiological properties such as intraluminal flow, vascularization, culture conditions optimized for long-term expansion, as well as inclusion of potentially supportive interstitial cell-types. Evidence shows that sorted proximal tubule-like cells from human iPSC-derived kidney organoids can be cultured for months, introducing the possibility to generate PB organoids for long-term tubule culture or scaling for large-scale screens using, for instance, bioreactor approaches. Additionally, bioengineered chips offer the possibility of highly controlled studies of pharmacological drug uptake. These chip technologies have further allowed for the introduction of nephrons with vasculature to model renal physiologies, and collectively these methods and the rapidly developing kidney organoid field contribute to the expanding toolkit available for kidney research and complement the developmental-mimicry approach.

### EXAMPLE ASPECTS

Example 1: A proximal-biased kidney organoid, comprising a kidney organoid exposed to a PI3 kinase (PI3K) inhibitor and comprising proximal tubule cells, wherein incubation with the PI3K inhibitor provides a proximal bias in the kidney organoid.

Example 2: The proximal-biased kidney organoid of any examples herein, particularly Example 1, wherein the PI3K inhibitor comprises LY294002, GDC-0941, or any combination thereof.

Example 3: The proximal-biased kidney organoid of any examples herein, particularly Examples 1-2, wherein the kidney organoid is exposed to the PI3K inhibitor for from about 12 hours to about 5 days.

Example 4: The proximal-biased kidney organoid of any examples herein, particularly Example 3, wherein the kidney organoid is exposed to the PI3K inhibitor for about 2 days.

Example 5: The proximal-biased kidney organoid of any examples herein, particularly Examples 1-4, wherein the kidney organoid is exposed to the PI3K inhibitor at a concentration of from about 1 μM to about 100 μM.

Example 6: The proximal-biased kidney organoid of any examples herein, particularly Example 5, wherein the kidney organoid is exposed to the PI3K inhibitor at a concentration of about 10 μM.

Example 7: The proximal-biased kidney organoid of any examples herein, particularly Examples 1-6, further comprising HNF4A⁺ cells.

Example 8: The proximal-biased kidney organoid of any examples herein, particularly Examples 1-7, further comprising an S-shaped body nephron.

Example 9: The proximal-biased kidney organoid of any examples herein, particularly Example 8, further comprising HNF4A⁺ cells localized in a medial segment of the S-shaped body nephron.

Example 10: The proximal-biased kidney organoid of any examples herein, particularly Examples 1-9, wherein the proximal-biased kidney organoid uptakes dextran and/or albumin.

Example 11: The proximal-biased kidney organoid of any examples herein, particularly Examples 1-10, wherein the proximal-biased kidney organoid demonstrates upregulation of KIM1, HAVCR1, HAVCR2, EGR1, STAT3, and/or SOX9⁺ in response to nephrotoxic injury.

Example 12: A method of producing the proximal-biased kidney organoid of any examples herein, particularly Examples 1-11, the method comprising incubating a plurality of precursor cells with a cell culture media, a PI3K inhibitor, and at least one compound for inducing kidney cell differentiation.

Example 13: The method of any examples herein, particularly Example 12, wherein the PI3K inhibitor comprises LY294002, GDC-0941, or any combination thereof.

Example 14: The method of any examples herein, particularly Examples 12-13, wherein the at least one compound for inducing kidney cell differentiation comprises CHIR99021.

Example 15: The method of any examples herein, particularly Examples 12-14, wherein the cell culture media is TeSR-E6.

Example 16: The method of any examples herein, particularly Examples 12-15, wherein the plurality of precursor cells comprise induced pluripotent stem cells (iPSCs), mesenchymal stem cells (MSCs), nephron progenitor cells, nephron precursor cells, or any combination thereof.

Example 17: The method of any examples herein, particularly Examples 12-16, wherein the PI3K inhibitor and the at least one compound for inducing kidney cell differentiation are introduced to the plurality of precursor cells simultaneously.

Example 18: The method of any examples herein, particularly Examples 12-16, wherein the PI3K inhibitor is introduced to the plurality of precursor cells after the at least one compound for inducing kidney cell differentiation is introduced to the plurality of precursor cells.

Example 19: The method of any examples herein, particularly Example 18, wherein the PI3K inhibitor is introduced to the plurality of precursor cells from about 5 days to about 15 days after the at least one compound for inducing kidney cell differentiation is introduced to the plurality of precursor cells.

Example 20: The method of any examples herein, particularly Examples 12-19, wherein the plurality of precursor cells are separately incubated with the PI3K inhibitor and with the at least one compound for inducing kidney cell differentiation.

Example 21: The method of any examples herein, particularly Examples 12-19, wherein the plurality of precursor cells are incubated with both the PI3K inhibitor and the at least one compound for inducing kidney cell differentiation for about 1 hour or more.

Example 22: The method of any examples herein, particularly Examples 12-21, wherein the plurality of precursor cells are incubated with the PI3K inhibitor for from about 12 hours to about 5 days.

Example 23: The method of any examples herein, particularly Example 22, wherein the plurality of precursor cells are incubated with the PI3K inhibitor for about 2 days.

Example 24: The method of any examples herein, particularly Examples 12-23, wherein the PI3K inhibitor is present in a concentration of from about 1 μM to about 100 μM.

Example 25: The method of any examples herein, particularly Example 24, wherein the PI3K inhibitor is present in a concentration of about 10 μM.

Example 26: The method of any examples herein, particularly Examples 12-25, wherein the plurality of precursor cells are incubated with at least one additional cell growth and/or differentiation compound.

Example 27: The method of any examples herein, particularly Example 26, wherein the at least one additional cell growth and/or differentiation compound comprises FGF, heparin, or a combination thereof.

Example 28: The method of any examples herein, particularly Examples 12-27, wherein the plurality of precursor cells are incubated with the cell culture media alone for from about 1 day to about 10 days.

Example 29: The method of any examples herein, particularly Example 28, wherein the plurality of precursor cells are incubated with the cell culture media alone after the plurality of precursor cells are incubated with the PI3K inhibitor and the at least one compound for inducing kidney cell differentiation.

Example 30: The method of any examples herein, particularly Examples 12-29, wherein the plurality of precursor cells are incubated for from about 15 days to about 30 days in total.

Example 31: A method of modeling a kidney disease, the method comprising: a) providing the proximal-biased kidney organoid of any examples herein, particularly Examples 1-11; and b) observing the proximal-biased kidney organoid over a period of time.

Example 32: The method of any examples herein, particularly Example 31, wherein the proximal-biased kidney organoid comprises healthy cells, and wherein step a) further comprises physically and/or chemically damaging the proximal-biased kidney organoid.

Example 33: The method of any examples herein, particularly Example 31, wherein the proximal-biased kidney organoid comprises diseased or abnormal cells.

Example 34: The method of any examples herein, particularly Example 33, wherein the proximal-biased kidney organoid comprises cells consistent with a renal tubule disease or disorder and/or a nephrotic disease or disorder.

Example 35: The method of any examples herein, particularly Examples 31-34, wherein the proximal-biased kidney organoid is derived from cells taken from a patient.

Example 36: The method of any examples herein, particularly Example 35, wherein the patient has a kidney disease or disorder.

Example 37: The method of any examples herein, particularly Examples 31-36, further comprising administering a therapeutic agent to the proximal-biased kidney organoid.

Example 38: The method of any examples herein, particularly Example 37, wherein the therapeutic agent comprises a small molecule, a chemotherapy agent, a biologic agent (e.g., a peptide or antibody), a nucleic acid, a therapeutic cell, a therapeutic treatment (e.g., surgery/ablation, radiation, etc.), or any combination thereof.

Example 39: The method of any examples herein, particularly Examples 31-38, wherein the proximal-biased kidney organoid is observed over a period of minutes, hours, days, weeks, or months.

Example 40: A method of screening for therapeutic agents that modulate injury to proximal tubule cells, the method comprising: a) providing the proximal-biased kidney organoid of any examples herein, particularly Examples 1-11; b) administering a therapeutic agent to the proximal-biased kidney organoid; and c) observing the proximal-biased kidney organoid over a period of time.

Example 41: The method of any examples herein, particularly Example 40, wherein step a) further comprises physically and/or chemically damaging the proximal-biased kidney organoid, and wherein the method is used to screen for therapeutic agents that treat or improve injury to proximal tubule cells.

Example 42: The method of any examples herein, particularly Example 40, wherein the proximal-biased kidney organoid comprises healthy cells, and wherein the method is used to screen for therapeutic agents that do not cause injury to proximal tubule cells.

Example 43: The method of any examples herein, particularly Example 40, further comprising, after step b) and before step c), physically and/or chemically damaging the proximal-biased kidney organoid, and wherein the method is used to screen for therapeutic agents that prevent or lessen injury to proximal tubule cells.

Example 44: The method of any examples herein, particularly Examples 40-43, wherein the therapeutic agent comprises a small molecule, a chemotherapy agent, a biologic agent (e.g., a peptide or antibody), a nucleic acid, a therapeutic cell, a therapeutic treatment (e.g., surgery/ablation, radiation, etc.), or any combination thereof.

Example 45: The method of any examples herein, particularly Examples 40-44, wherein the proximal-biased kidney organoid is observed over a period of minutes, hours, days, weeks, or months.

The following patents, applications and publications as listed below and throughout this document are hereby incorporated by reference in their entirety herein.

1. A proximal-biased kidney organoid, comprising a kidney organoid exposed to a PI3 kinase (PI3K) inhibitor and comprising proximal tubule cells, wherein incubation with the PI3K inhibitor provides a proximal bias in the kidney organoid.

2. The proximal-biased kidney organoid of claim 1, wherein the PI3K inhibitor comprises LY294002, GDC-0941, or any combination thereof.

3. The proximal-biased kidney organoid of claim 1, further comprising HNF4A⁺ cells.

4. The proximal-biased kidney organoid of claim 3, further comprising an S-shaped body nephron; and
wherein the HNF4A⁺ cells are localized in a medial segment of the S-shaped body nephron.

5. The proximal-biased kidney organoid of claim 1, wherein the proximal-biased kidney organoid uptakes dextran and/or albumin.

6. The proximal-biased kidney organoid of any claim 1, wherein the proximal-biased kidney organoid demonstrates upregulation of KIM1, HAVCR1, HAVCR2, EGR1, STAT3, and/or SOX9⁺ in response to nephrotoxic injury.

7. A method of producing the proximal-biased kidney organoid of claim 1, the method comprising incubating a plurality of precursor cells with a cell culture media, a PI3K inhibitor, and at least one compound for inducing kidney cell differentiation.

8. The method of claim 7, wherein the PI3K inhibitor comprises LY294002, GDC-0941, or any combination thereof.

9. The method of claim 7, wherein the at least one compound for inducing kidney cell differentiation comprises CHIR99021.

10. The method of claim 7, wherein the PI3K inhibitor and the at least one compound for inducing kidney cell differentiation are introduced to the plurality of precursor cells simultaneously; or
wherein the PI3K inhibitor is introduced to the plurality of precursor cells after the at least one compound for inducing kidney cell differentiation is introduced to the plurality of precursor cells.

11. The method of claim 7, wherein the plurality of precursor cells are separately incubated with the PI3K inhibitor and with the at least one compound for inducing kidney cell differentiation; or
wherein the plurality of precursor cells are incubated with both the PI3K inhibitor and the at least one compound for inducing kidney cell differentiation for about 1 hour or more.

12. The method of claim 7, wherein the plurality of precursor cells are incubated with the PI3K inhibitor for from about 12 hours to about 5 days.

13. The method of claim 7, wherein the PI3K inhibitor is present in a concentration of from about 1 μM to about 100 μM.

14. The method of claim 7, wherein the plurality of precursor cells are incubated with the cell culture media alone for from about 1 day to about 10 days.

15. A method of modeling a kidney disease, the method comprising:
a) providing the proximal-biased kidney organoid of claim 1; and
b) observing the proximal-biased kidney organoid over a period of time.

16. The method of claim 15, wherein the proximal-biased kidney organoid comprises healthy cells, and wherein step a) further comprises physically and/or chemically damaging the proximal-biased kidney organoid.

17. The method of claim 15, wherein the proximal-biased kidney organoid comprises diseased or abnormal cells.

18. The method of claim 15, further comprising administering a therapeutic agent to the proximal-biased kidney organoid.

19. A method of screening for therapeutic agents that modulate injury to proximal tubule cells, the method comprising:
a) providing the proximal-biased kidney organoid of claim 1;
b) administering a therapeutic agent to the proximal-biased kidney organoid; and
c) observing the proximal-biased kidney organoid over a period of time.

20. The method of claim 19, wherein step a) further comprises physically and/or chemically damaging the proximal-biased kidney organoid, and wherein the method is used to screen for therapeutic agents that treat or improve injury to proximal tubule cells.
